(19) United States
(12) Patent Application Publication
(10) Pub. No.: US 2023/0340111 A1
(43) Pub. Date: Oct. 26, 2023

(54) ANTIBODY FRAGMENT AGAINST FOLR1

(71) Applicant: Precirix N.V., Brussels (BE)

(72) Inventors: Tony LAHOUTTE, Brussels (BE); Nick DEVOOGDT, Brussels (BE); Matthias D′HUYVETTER, Brussels (BE); Sam MASSA, Brussels (BE)

(21) Appl. No.: 18/154,494

(22) Filed: Jan. 13, 2023

Related U.S. Application Data
(63) Continuation of application No. PCT/EP2021/069469, filed on Jul. 13, 2021.

(30) Foreign Application Priority Data
Jul. 13, 2020 (EP) 20185584.8

(51) Int. Cl. A61K 51/10; C07K 16/28; A61P 35/00

(52) U.S. Cl. CPC C07K 16/28; A61K 51/1027; A61K 51/1093; A61P 35/00; A61K 2039/505; C07K 2317/33; C07K 2317/34; C07K 2317/569; C07K 2317/92

(57) ABSTRACT

The present invention relates to the field of antibody fragments, which specifically binds an epitope of human folate receptor alpha (FOLR1) and which may be linked to an entity such as a moiety. The antibody fragment and the compound formed may be used for therapy or diagnostic purposes.

### RELATED APPLICATIONS

This application is a continuation of PCT International Application PCT/EP2021/069469 (published as WO2022/013225), filed Jul. 13, 2021, which claims priority to EP Application No. 20185584.8, filed Jul. 13, 2020, the entirety of each of which is hereby incorporated by reference.

### SEQUENCE LISTING

The content of the electronically submitted Sequence Listing XML (Name: PREC-003US (195995); Size: 98,179 bytes; Created: Jan. 13, 2023) is herein incorporated by reference in its entirety.

### FIELD OF THE INVENTION

The present invention relates to the field of antibody fragments, which specifically binds an epitope of human folate receptor alpha (FOLR1) and which may be linked to an entity such as a moiety. Depending on the application of said antibody fragment, the moiety may be a molecule to be delivered to the central nervous system or a label which may be a radionuclide. In particular, the molecule is a medicament acting on the brain. Alternatively, the present invention relates to labelled antibody fragments for use in the prevention and/or treatment of cancer.

### BACKGROUND

Cancers figure among the leading causes of morbidity and mortality worldwide. There is a continuous need for improved therapies combatting cancer while minimizing side effects.

Folate receptor alpha (FOLR1), also known as folate receptor 1 or folate binding protein, is overexpressed in vast majority of ovarian cancers, as well as in many uterine, endometrial, pancreatic, renal, lung, brain and breast cancers, while the expression of FOLR1 on normal tissues is restricted to the apical membrane of epithelial cells in the kidney proximal tubules, alveolar pneumocytes of the lung, bladder, testes, choroid plexus, and thyroid (Weitman et al. (1992) Cancer Res 52: 3396-3401; Antony (1996) Annu Rev Nutr 16: 501-521; Kalli et al. (2008) Gynecol Oncol 108: 619-626).

Anti-FOLR1 antibodies have been examined as potential anti-cancer drugs. Murine monoclonal antibodies Mov18 and Mov19 were isolated in the late 1980s (Miotti S et al. (1987) Int J Cancer 39: 297-303).

MORAb003, a humanized form of the murine monoclonal anti-FOLR1 antibody LK26 was evaluated pre-clinically as a non-modified antibody that is an antagonist of human FOLR1 (Ebel et al. (2007) Cancer Immun 7:6) and as a conjugate with the ¹¹¹In radionuclide as imaging agent (Smith-Jones et al. (2008) Nucl Med Biol35: 343-351), and is currently undergoing clinical trials as a non-modified antibody (Armstrong et al. (2008) J. Clin. Oncol. 26: abstract 5500). Mirvetuximab is a humanized antibody derived from Mov19 which is undergoing clinical trial for platinum-resistant ovarian cancer (Noore, K et al, Future Oncol, (2018), 14: 123-136).

So far no antibody targeting FOLR1 had been approved as therapeutic antibody.

As such, there is a need in the art for further antibodies that target human FOLR1.

### LEGEND TO THE DRAWINGS

FIG. 1. Schematic illustration of the different elements in the targeting vector to knock-in the human folr1 cDNA in exon 4 of the mouse folr1 gene by homologous recombination.

FIG. 2. Signal-to background ratios of His₆-tagged VHHs A1, A2, A3, A4 and A5 binding to recombinant human FOLR1, FOLR2, FOLR3 and recombinant murine FOLR1, as determined by ELISA.

FIG. 3. ΔMFI values of the binding of His₆-tagged VHHs A1, A2, A3, A4 and A5 to human FOLR1^{pos} SKOV-3 cells, in the absence or presence of a 100-fold molar excess of folic acid, as determined by flow cytometry analysis.

FIG. 4. The concentration-response curves for ^{99m}Tc-labeled VHHs A1, A2, A3, A4 and A5 with corresponding EC50 obtained on human FOLR1^{pos} SKOV-3 cancer cells. Data presented as mean ± SD (n = 3).

FIG. 5. The concentration-response curves for ¹³¹I-labeled, His₆-tagged VHHs A1, A2, A3, A4 with corresponding EC50 values obtained on human FOLR1^{pos} SKOV-3 cancer cells. Data presented as mean ± SD (n = 3).

FIG. 6. The concentration-response curves for ¹³¹I-labeled untagged VHHs A1, A2, A3 with corresponding EC50 values obtained on human FOLR1^{pos} SKOV-3 cancer cells. Data presented as mean ± SD (n = 3).

FIG. 7. The cellular retention of ¹³¹I-labeled His₆-tagged VHHs A1, A2, A3 and A4 up to 24h obtained on human FOLR1^{pos} SKOV-3 cancer cells. Data presented as mean ± SD (n = 3).

FIG. 8. The cellular retention of ¹³¹I-labeled, untagged VHHs A1, A2, A3 up to 24h obtained on human FOLR1^{pos} SKOV-3 cancer cells. Data presented as mean ± SD (n = 3).

### DETAILED DESCRIPTION OF THE INVENTION

### Antibody Fragment

In a first aspect of the invention, there is provided an antibody fragment which specifically binds human folate receptor alpha (FOLR1).

In an embodiment, this antibody fragment specifically binds human folate receptor alpha (FOLR1, which is represented by SEQ ID NO:1), but does neither specifically bind human folate receptor beta (FOLR2) nor human folate receptor gamma (FOLR3) and is further characterized by an additional (structural) feature as defined herein (such as first, second, third and/or fourth structural features) .

In an embodiment, this antibody fragment specifically binds human folate receptor alpha (FOLR1, which is represented by SEQ ID NO:1), but does neither specifically bind murine FOLR1 nor human folate receptor beta (FOLR2) nor human folate receptor gamma (FOLR3) and is further characterized by an additional (structural) feature as defined herein (such as first, second, third and/or fourth structural features).

Throughout the application, the antibody fragment of the invention may specifically bind murine FOLR1. Alternatively, it may not specifically bind murine FOLR1.

In an embodiment, this antibody fragment specifically binds human folate receptor alpha (FOLR1, which is represented by SEQ ID NO:1), but does neither specifically bind murine FOLR1 nor human folate receptor beta (FOLR2) nor human folate receptor gamma (FOLR3) (or this antibody fragment specifically binds human FOLR1 but does neither specifically bind human FOLR2 nor human FOLR3) and fulfils at least one of the following:
a. the epitope is comprised within amino acid 25 to 233 of SEQ ID NO:1,
b. at least amino acid C89, G90, E91, M92, A93, P94, E140, Q141, W142, W143, E144, D145, C146, R147, T148, S149, Y150, Q176, P177, F178, H179, F180, Y181, F182, P183 and/or T184 of SEQ ID NO:1 interacts with said antibody fragment,
c. said antibody fragment is represented by an amino acid sequence that comprises an amino acid sequence having at least 60% sequence identity with at least one of SEQ ID NO:2, 3, 4, 5, 6, 7, 8 or 9 or a portion thereof.

In an embodiment of this aspect, there is provided an antibody fragment that specifically binds an epitope of human folate receptor alpha (FOLR1), but does neither bind murine FOLR1 nor human folate receptor beta (FOLR2) nor human folate receptor gamma (FOLR3) (or this antibody fragment specifically binds human FOLR1 but does neither specifically bind human FOLR2 nor human FOLR3) and said antibody fragment fulfils a) and/or b):
a. the epitope is comprised within amino acid 25 to 233 of SEQ ID NO:1 and the antibody fragment specifically binds to the following amino acids of SEQ ID NO:1:
   1) at least one, or at least two, or at least three amino acids selected from C89, G90, E91, M92, A93, or P94, and/or
   2) at least one, or at least two, or at least three amino acids selected from E140, Q141, W142, W143, E144, D145, C146, R147, T148, S149, or Y150, and/or
   3) at least one, or at least two, or at least three amino acids selected from Q176, P177, F178, H179, F180, Y181, F182, P183, or T184,
b. the antibody fragment is represented by an amino acid sequence that comprises an amino acid sequence having at least 63% sequence identity with at least one of SEQ ID NO:8, 9, 3, 4, 5, 6, or 7 over the full length of said sequence or over at least 50% of the length of said sequence.

In an embodiment of this aspect, there is provided an antibody fragment, preferably as defined above, that specifically binds an epitope of human folate receptor alpha (FOLR1), but does neither bind murine FOLR1 nor human folate receptor beta (FOLR2) nor human folate receptor gamma (FOLR3), (or this antibody fragment specifically binds human FOLR1 but does neither specifically bind human FOLR2 nor human FOLR3), wherein the epitope is comprised within amino acid 25 to 233 of SEQ ID NO:1 and the antibody fragment specifically binds to the following amino acids of SEQ ID NO:1:
1) at least one, or at least two, or at least three amino acids selected from C89, G90, E91, M92, A93, or P94, and
2) at least one, or at least two, or at least three amino acids selected from E140, Q141, W142, W143, E144, D145, C146, R147, T148, S149, or Y150, and
3) at least one, or at least two, or at least three amino acids selected from Q176, P177, F178, H179, F180, Y181, F182, P183, or T184.

In an embodiment of this aspect, there is provided an antibody fragment, preferably as defined above, that specifically binds an epitope of human folate receptor alpha (FOLR1), but does neither bind murine FOLR1 nor human folate receptor beta (FOLR2) nor human folate receptor gamma (FOLR3), wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence.

In an embodiment of this aspect, there is provided an antibody fragment, preferably as defined above, that specifically binds an epitope of human folate receptor alpha (FOLR1), but does neither bind human folate receptor beta (FOLR2) nor human folate receptor gamma (FOLR3), wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence.

TABLE 1

Amino acid sequence of human FOLR1 (Uniprot entry P15328)

| | |
|---|---|
| SEQ ID NO:1 | MAQRMTTQLLLLLVWVAVVGEAQTRIAWARTELLNVCMNAKHHKEKPG PEDKLHEQCRPWRKNACCSTNTSQEAHKDVSYLYRFNWNHCGEMAPA CKRHFIQDTCLYECSPNLGPWIQQVDQSWRKERVLNVPLCKEDCEQW WEDCRTSYTCKSNWHKGWNWTSGFNKCAVGAACQPFHFYFPTPTVL |
| | CNEIWTHSYKVSNYSRGSGRCIQMWFDPAQGNPNEEVARFYAAAMSG AGPWAAWPFLLSLALMLLWLLS |

Within the context of the invention, Folate receptor alpha also known as folate receptor 1 or folate binding protein is denominated using the abbreviation FOLR1 or FR-alpha at the protein and at the gene levels. Officially, FR-alpha is the recommended protein name from Uniprot and FOLR1 is used for the gene name.

Within the context of the invention, the term “antibody fragment” refers to any fragment of an antibody or immunoglobulin. In an embodiment, the antibody fragment is a single-domain antibody fragment. In an embodiment, the antibody fragment is a heavy chain variable domain derived from a heavy chain antibody (VHH) or a fragment thereof. In a preferred embodiment, a single-domain antibody fragment is a VHH or a fragment thereof: the heavy chain variable domains derived from heavy chain antibodies (i.e., the VHH’s) as disclosed herein consist of a single polypeptide chain. Within the context of the application, the expression “antibody fragment” may be replaced by “single-domain antibody fragment” or by “VHH” or by “a fragment of a VHH” or by “a functional fragment of a VHH”. Preferably a fragment of an antibody or of a VHH is a functional fragment as it exhibits at least an activity of the antibody or of the VHH to some extent. “Some extent” may mean at least 40%, 50%, 60%, 70%, 80%, 90%, 95%, 100% or more. A preferred activity of the antibody fragment, VHH or a fragment of a VHH is the specific binding to human FOLR1. The “specific binding to human FOLR1” has been defined later herein. At the end of the description, a more detailed definition of “antibody”, “antibody fragment”, “agonist” “antagonist”, “variants of antibody fragment” is provided.

More particularly, the VHH’s or fragments thereof disclosed herein are derived from an innate or adaptive immune system, preferably from a protein of an innate or adaptive immune system. Still more particularly, the VHH’s disclosed herein may comprise 4 framework regions (FR) and 3 complementary determining regions (CDR), or any suitable fragment thereof (which will then usually contain at least some of the amino acid residues that form at least one of the CDR). In particular, the VHH’s disclosed herein are easy to produce at high yield, preferably in a microbial recombinant expression system, and convenient to isolate and/or purify subsequently.

According to particular embodiments described in more details later herein, the invention provides an antibody fragment particularly suited for binding to human FOLR1. In an embodiment, the antibody fragment binds part of the extracellular domain of human FOLR1. Human FOLR1 is quite attractive to be targeted as it may allow blood brain barrier (BBB) and/or blood-cerebrospinal fluid barrier (BCSFB) entrance for a molecule coupled to it (Alam et al. (2020) Trends Pharmacol Sci 41(5):349-361; Strazielle (2016) Curr Pharm Des 22(35):5463-5476). Alternatively human FOLR1 is specifically expressed and more specifically overexpressed in some cancer cells (such as ovarian, endometrial, lung or breast cancer as disclosed later herein) and poorly expressed in healthy cells. Human FOLR1 may therefore be considered as a tumour antigen or a cancer cell antigen and may therefore be used as diagnostic and/or therapeutic agent (i.e., theranostic agent) (Scaranti et al. (2020) Nat Rev Clin Oncol 17(6):349-359; Xu et al. (2017) J Control Rel 252:73-82).

The antibody fragment of the invention may comprise CDR sequences of antibodies (or may be based on and/or derived from such CDR sequences, as further described herein), they will also generally be referred to herein as ‘CDR sequences’ (i.e., as CDR1 sequences, CDR2 sequences and CDR3 sequences, respectively). In an embodiment, the VHH’s as disclosed herein comprise at least one amino acid sequence that is chosen from the group consisting of the CDR1 sequences, CDR2 sequences and CDR3 sequences that are described herein. Thus, in particular embodiments, the present invention provides heavy chain variable domains derived from heavy chain antibodies with the (general) structure:

FR1 - CDR1 - FR2 - CDR2 - FR3 - CDR3 - FR4

Within the context of the invention the IMGT nomenclature is used to define the FR (framework regions) FR1, FR2, FR3 and FR4 and corresponding CDR regions CDR1, CDR2, and CDR3. The definition of the IMGT nomenclature used is provided later herein in the general part dedicated to the definition of the invention. It should however be noted that the invention in its broadest sense is not limited to a specific structural role or function that these stretches of amino acid residues may have in the heavy chain variable domains as disclosed herein, as long as these stretches of amino acid residues allow the variable domains as disclosed herein to specifically bind to human FOLR1. Thus, generally, the invention in its broadest sense relates to an antibody fragment, such as a single-domain antibody fragment, preferably a VHH or a fragment thereof, which can be coupled to an entity such as a moiety. Within the context of the invention, an antibody fragment, preferably a VHH or a fragment thereof coupled to an entity such as a moiety may be called a compound.

This moiety can be a molecule that is to be delivered to the central nervous system, preferably the brain. It is assumed that the antibody fragment, preferably a VHH or a fragment thereof can target said molecule to the brain. Alternatively, this moiety can be a label. The label may be a radionuclide. Alternatively, the label may be non-radioactive. When the compound of the invention comprises a label, it may be called a labelled compound. A radioactive labelled compound is preferably used for treating a cancer associated with the expression of human FOLR1 on a cancer cell. A non-radioactive labelled compound is preferably used in a diagnostic application as later disclosed herein.

The antibody fragment such as a single-domain antibody fragment, preferably a VHH or fragment thereof may be characterized by a functional feature and/or by a structural feature. Examples of structural features are sequence related and examples of functional features are related to an activity of said antibody fragment. An activity may be a specific binding activity. An activity may also be the absence of a specific binding activity or the absence of the detection of a specific binding activity.

Specific binding of an antibody fragment can be determined in any suitable manner known per se, including, for example biopanning, Scatchard analysis and/or competitive binding assays, such as radioimmunoassays (RIA), enzyme immunoassays (EIA) (also called Enzyme-Linked Immuno Sorbent Assay, ELISA), sandwich competition assays, or Surface Plasmon Resonance (SPR) and the different variants thereof known in the art. Each of these assays may be carried out in vitro using the human FOLR1 recombinant protein which may be immobilised on a support or in solution. In an embodiment, the antibody fragment, preferably a VHH or fragment thereof is immobilised on a support, preferably as carried out in example 4e. In another embodiment, the human FOLR1 recombinant protein is immobilised on a support (i.e., human FOLR1 being the immobilized ligand), preferably as carried out in example 4d. The immobilisation of the human FOLR1 is preferred as the immobilization of the antibody fragment, preferably a VHH or fragment thereof may occur at random and in any orientation, partially obscuring the VHH paratope and may therefore alter the value of the kinetic parameter assessed. Alternatively, under some specific circumstances, some of these assays may be carried out in vitro using cells that express human FOLR1. Such cells may endogenously express or overexpress human FOLR1. The assessment is usually carried out in vitro in a culture medium or in PBS or in a suitable medium or buffer. A preferred cell is SKOV3 or OVCAR3.

Alternatively, the binding of the antibody fragment may be assessed in vivo in an animal expressing human FOLR1. In such setting, the antibody fragment is preferably labelled, more preferably radiolabelled and an imaging technique is used. A preferred imaging technique is SPECT/CT or PET/CT. SKOV3 cells overexpressing human FOLR1 may also be xenografted into the animal. It is also possible to use the human FOLR1 knock-in mouse of the invention expressing human FOLR1.

The wording “in vitro” is therefore used herein in the context of a cell-free assay when the FOLR1 recombinant protein is immobilized on a support or in the context of a cell in culture. As opposed to that, the wording “in vivo” or “ex vivo” is used herein in the context of a non-human animal or a tissue or organ of this non-human animal. Usually “ex vivo” is used when a quantification is carried out on a tissue or organ of a non-human animal and “in vivo” is used when a quantification via an imaging method is carried out on a human animal. Usually “binding” is assessed in vitro conditions and is further confirmed in vivo conditions.

In the in vitro, in vivo and ex vivo assays disclosed herein, it is preferred to use a negative control. It is also possible to assess the specific binding to human FOLR1 in the presence of other antigens as explained later herein. In the experimental part, several assays have been used to assess the specific binding of the antibody fragment of the invention: example 4 wherein ELISA and SPR have been used and example 5 wherein the binding has been assessed in the human FOLR1 knock-in mouse or in a mouse comprising SKOV3 cells overexpressing human FOLR1 using SPECT/CT imaging and/or ex vivo gamma counting of dissected tissues.

The term “affinity”, “specific binding”, “binding”, “binding activity” or “specific binding activity”, as used herein, refers to the degree to which an antibody fragment such as a single-domain antibody fragment preferably a VHH, or a fragment thereof binds to human FOLR1 so as to shift the equilibrium of human FOLR1 and the antibody fragment toward the presence of a complex formed by their binding. The binding may be assessed using SPR. Thus, for example, where human FOLR1 and the antibody fragment are combined in relatively equal concentration, the antibody fragment of high affinity will bind to the available human FOLR1 so as to shift the equilibrium toward high concentration of the resulting complex. The equilibrium dissociation constant (K_{D}) is commonly used to describe the affinity between the protein binding domain (antibody fragment) and the antigenic target (human FOLR1). Typically, the equilibrium dissociation constant is less than 10⁻⁷ M. Preferably, the equilibrium dissociation constant is less than 10⁻⁸ M, or less than 10⁻⁹ M, or more preferably, ranged from 10⁻⁹ M and 10⁻¹¹ M.

Any antibody fragment as disclosed herein is preferably such that it specifically binds (as defined herein) to human FOLR1 with an equilibrium dissociation constant (K_{D}) ranged from 10⁻⁹ to 10⁻¹¹ moles/liter or from 10⁻¹⁰ to 10⁻¹¹ moles/liter, preferably assessed using SPR.

The ‘specificity’ of an antibody fragment such as a single-domain antibody fragment, preferably a VHH, or fragments thereof as disclosed herein can be determined based on affinity and/or avidity. The ‘affinity’ of an antibody fragment as disclosed herein is represented by the equilibrium constant for the dissociation of the antibody fragment as disclosed herein and human FOLR1 to which it binds. The lower the K_{D} value, the stronger the binding strength between the antibody fragment as disclosed herein and the target protein of interest to which it binds. Alternatively, the affinity can also be expressed in terms of the equilibrium association constant (K_{A}), which corresponds to 1/Ko. The binding affinity of an antibody fragment as disclosed herein can be determined in a manner known to the skilled person, depending on the specific target protein of interest. The ‘avidity’ of an antibody fragment as disclosed herein is the measure of the strength of binding between the antibody fragment as disclosed herein and the pertinent target protein of interest. Avidity is related to both the affinity between a binding site on the target protein of interest and a binding site on the antibody fragment as disclosed herein and the number of pertinent binding sites present on the antibody fragment as disclosed herein. Preferred antibody fragments of the invention such as VHHs have only one single-domain and therefore only one single binding site. The affinity exhibited by such a single-domain antibody fragment is in the sub-nano molar range and is therefore quite exceptional in view of the presence of a single binding site. A K_{D} value greater than about 1 millimolar is generally considered to indicate non-binding or non-specific binding. It is generally known in the art that the K_{D} can also be expressed as the ratio of the dissociation rate constant of a complex, denoted as k_{off} or k_{d} (expressed in seconds⁻¹ or s⁻¹), to the rate constant of its association, denoted kₒₙ or kₐ (expressed in molar⁻¹ seconds⁻¹ or M⁻¹ s⁻¹). In particular, the antibody fragment as disclosed herein will bind to the target protein of interest (i.e., human FOLR1) with a k_{off} ranging from 0.1 and 0.00001 s⁻¹, or ranging from 10⁻² to 10⁻⁴ s⁻¹ or from 10⁻³ to 10⁻⁴ s⁻¹ and/or a kₒₙ ranging from 1,000 and 10,000,000 M⁻¹ s⁻¹ or ranging from 10⁴ to 10⁶ M⁻¹s⁻¹ or from 10⁵ to 10⁶ M⁻¹s⁻¹. Binding affinities, k_{off} and kₒₙ rates may be determined by means of methods known to the person skilled in the art, for example ELISA methods, isothermal titration calorimetry, SPR, bio-layer interferometry, fluorescence-activated cell sorting analysis, and the more. Preferably SPR is used as illustrated in example 4c.

In a preferred embodiment, the antibody fragment as disclosed herein specifically binds to human FOLR1 with a k_{off} ranging from 0.1 and 0.00001 s⁻¹, or ranging from 10⁻² to 10⁻⁴ s⁻¹ or from 10⁻³ to 10⁻⁴ s⁻ ¹, preferably assessed using SPR, more preferably using SPR with the human FOLR1 being the immobilized ligand (such as in example 4d).

In a preferred embodiment, an antibody fragment as disclosed herein is such that it specifically binds (as defined herein) to human FOLR1 with a K_{D} ranged from 10⁻⁹ to 10⁻¹¹ moles/liter and/or a k_{off} ranging from 10⁻⁴ to 10⁻² s⁻¹ preferably assessed using SPR, more preferably with a K_{D} ranged from 10⁻⁹ to 10⁻¹¹ moles/liter and a k_{off} ranging from 10⁻⁴ to 10⁻² s⁻¹. In a more preferred embodiment, SPR is used with the human FOLR1 being the immobilized ligand (such as in example 4d).

The experimental part (see examples 4d and 4e) demonstrates that the antibody fragment of the invention, preferably a VHH or a fragment thereof exhibits attractive kinetic characteristics, more attractive than the ones of the antibodies known in US 2014/0205610.

Accordingly, an antibody fragment such as a single-domain antibody fragment (preferably a VHH or a fragment thereof), as disclosed herein is said to ‘specifically bind to’ human FOLR1 when that antibody fragment has affinity for, specificity for and/or is specifically directed against that target (or for at least one part or fragment thereof).

In respect of the antibody fragment such as a VHH or fragments thereof, as disclosed herein, the terms ‘binding region’, ‘binding site’ or ‘interaction site’ present on the antibody fragment as disclosed herein shall herein have the meaning of a particular site, part, locus, domain or stretch of amino acid residues present on the antibody fragment as disclosed herein that is responsible for binding or specific binding to human FOLR1. This binding region present on the antibody fragment is called a paratope. Such binding region comprises, consists or essentially consists of specific amino acid residues from the amino acid sequence as disclosed herein of the antibody fragment which are in contact with human FOLR1. The region or part or discrete amino acids of the extracellular domain of the human FOLR1 that is in contact with said antibody fragment may be called an epitope and are defined later herein.

The terms ‘specifically bind’ and ‘specific binding’, as used herein, generally refers to the ability of a polypeptide, in particular an immunoglobulin, such as an antibody, or an antibody fragment, such as a single-domain antibody fragment preferably a VHH or fragments thereof, to preferentially bind to a particular antigen such as human FOLR1. Such an antibody fragment may also be identified as an antibody fragment raised against human FOLR1.

The binding to human FOLR1 may be assessed in a homogeneous mixture of different antigens. In certain embodiments, a specific binding interaction will discriminate between desirable and undesirable antigens in a sample, in some embodiments more than about 10 to 100-fold or more (e.g., more than about 1000- or 10,000-fold).

In an embodiment, the binding may be assessed in vitro using cells expressing human FOLR1, and optionally in vivo or ex vivo as earlier defined herein. These cells may be human cells and expressing endogenous human FOLR1. Alternatively, these cells may overexpress human FOLR1. Cells overexpressing human FOLR1 may be human or non-human cells. Preferred cells are SKOV3 and OVCAR3.

In an embodiment, the antibody fragment such as single-domain antibody fragment, preferably a VHH or fragments thereof, specifically binds to human FOLR1 and does not specifically bind to murine FOLR1 or to human FOLR2 or to human FOLR3. This assessment is preferably carried out using ELISA or SPR.

It is also expected that the antibody fragment such as single-domain antibody fragment, preferably the VHH or a fragment thereof of the invention will bind to a number of naturally occurring or synthetic analogs, variants, mutants, alleles, parts and fragments of human FOLR1.

In an embodiment, the antibody fragment, preferably the VHH or a fragment thereof of the invention will specifically bind to at least those analogs, variants, mutants, alleles, naturally occurring, synthetic analogs, parts and fragments of human FOLR1 that (still) contain the epitope of the (natural/wild-type) antigen to which the antibody fragment binds. The epitope of human FOLR1 for the antibody fragment of the invention is comprised within amino acid 25 to 233 of SEQ ID NO:1.

In an embodiment, at least one of the following amino acids of this part of SEQ ID NO:1 is bound by the antibody fragment: C89, G90, E91, M92, A93, P94, E140, Q141, W142, W143, E144, D145, C146, R147, T148, S149, Y150, Q176, P177, F178, H179, F180, Y181, F182, P183 and/or T184 of SEQ ID NO:1.

In another embodiment, the antibody fragment specifically binds to the following amino acids of SEQ ID NO:1:
1) at least one, or at least two, or at least three amino acids selected from C89, G90, E91, M92, A93, or P94, and/or
2) at least one, or at least two, or at least three amino acids selected from E140, Q141, W142, W143, E144, D145, C146, R147, T148, S149, or Y150, and/or
3) at least one, or at least two, or at least three amino acids selected from Q176, P177, F178, H179, F180, Y181, F182, P183, or T184.

In another embodiment, the antibody fragment specifically binds to the following amino acids of SEQ ID NO:1:
1) at least one, or at least two, or at least three amino acids selected from C89, G90, E91, M92, A93, or P94, and
2) at least one, or at least two, or at least three amino acids selected from E140, Q141, W142, W143, E144, D145, C146, R147, T148, S149, or Y150, and
3) at least one, or at least two, or at least three amino acids selected from Q176, P177, F178, H179, F180, Y181, F182, P183, or T184.

In another embodiment, the antibody fragment specifically binds to the following amino acids of SEQ ID NO:1:
1) at least two, or at least three amino acids selected from C89, G90, E91, M92, A93, or P94, and/or
2) at least two, or at least three amino acids selected from E140, Q141, W142, W143, E144, D145, C146, R147, T148, S149, or Y150, and/or
3) at least two, or at least three amino acids selected from Q176, P177, F178, H179, F180, Y181, F182, P183, or T184.

In another embodiment, the antibody fragment specifically binds to the following amino acids of SEQ ID NO:1:
1) at least two, or at least three amino acids selected from C89, G90, E91, M92, A93, or P94, and
2) at least two, or at least three amino acids selected from E140, Q141, W142, W143, E144, D145, C146, R147, T148, S149, or Y150, and
3) at least two, or at least three amino acids selected from Q176, P177, F178, H179, F180, Y181, F182, P183, or T184.

In another embodiment, the antibody fragment specifically binds to the following amino acids of SEQ ID NO:1:
1) at least three amino acids selected from C89, G90, E91, M92, A93, or P94, and/or
2) at least three amino acids selected from E140, Q141, W142, W143, E144, D145, C146, R147, T148, S149, or Y150, and/or
3) or at least three amino acids selected from Q176, P177, F178, H179, F180, Y181, F182, P183, or T184.

In an embodiment, the antibody fragment specifically binds to the following amino acids of SEQ ID NO:1:
1) at least three amino acids selected from C89, G90, E91, M92, A93, or P94, and
2) at least three amino acids selected from E140, Q141, W142, W143, E144, D145, C146, R147, T148, S149, or Y150, and
3) at least one, or at least two, or at least three amino acids selected from Q176, P177, F178, H179, F180, Y181, F182, P183, or T184.

In another embodiment, the following amino acids of amino acids 25 to 233 of SEQ ID NO:1 are bound by the antibody fragment: C89, G90, E91, M92, A93, P94, E140, Q141, W142, W143, E144, D145, C146, R147, T148, S149, Y150, Q176, P177, F178, H179, F180, Y181, F182, P183 and T184.

In this context, an amino acid of human FOLR1 may be bound by the antibody fragment of the invention when said amino acid belongs to the epitope of the antibody fragment.

An antibody fragment such as a single-domain antibody fragment preferably a VHH or fragments thereof, as disclosed herein is said to be ‘specific for a first target antigen of interest (i.e. human FOLR1) as opposed to a second target antigen of interest (i.e. murine FOLR1, human FOLR2, and/or human FOLR3) or (i.e. human FOLR2 and human FOLR3) when it binds to the first target antigen of interest with an affinity that is at least 5 times, such as at least 10 times, such as at least 100 times, and preferably at least 1000 times higher than the affinity with which that antibody fragment as disclosed herein binds to the second target antigen of interest. Accordingly, in certain embodiments, when an antibody fragment as disclosed herein is said to be ‘specific for’ a first target antigen of interest as opposed to a second target antigen of interest, it may specifically bind to (as defined herein) the first target antigen of interest, but not to the second target antigen of interest. Within the context of the invention, an antibody fragment specifically binds an epitope of human folate receptor alpha (FOLR1) and it does neither specifically bind murine FOLR1 nor human folate receptor beta (FOLR2) or gamma (FOLR3). In an embodiment, the antibody fragment specifically binds an epitope of human FOLR1 and it does not specifically binds human FOLR2 nor human FOLR3. In an embodiment, the antibody fragment additionally does not specifically bind murine folate receptor beta (FOLR2).

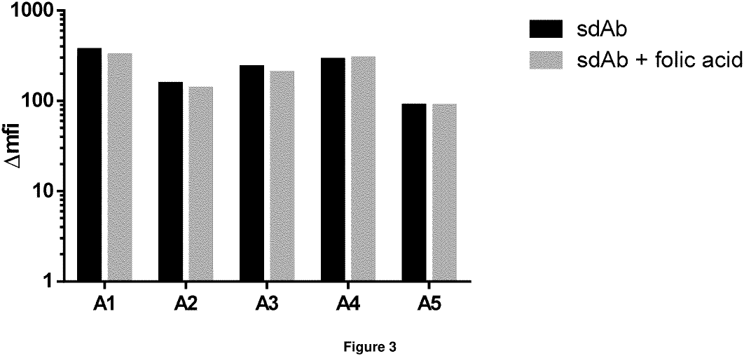

The terms ‘competing (with)’, ‘cross-blocking’, ‘cross-binding’ and ‘cross-inhibiting’ as used interchangeably herein, generally refer to an antibody fragment such as a VHH, as disclosed herein that can interfere with the binding of other antibody or other single-domain antibody fragment or other molecule as disclosed herein to human FOLR1, as measured using a suitable in vitro or in vivo assay. A preferred cell line used for testing the in vitro binding to human FOLR1 is SKOV3 expressing human FOLR1. Such a cell has been used in the experimental part (see example 4b). Thus, more particularly, ‘competing (with)’, ‘cross-blocking’, ‘cross-binding’ and ‘cross-inhibiting’ using an antibody fragment as disclosed herein may mean interfering with or competing with the binding of another antibody or single-domain antibody fragment as disclosed herein with human FOLR1, thereby reducing that binding by at least 10% but preferably at least 20%, for example by at least 50%, at least 60%, at least 70%, at least 80%, at least 90%, at least 95% or more, as measured using a suitable in vitro, cellular or in vivo assay, compared to the binding of that other single-domain antibody fragment as disclosed herein with human FOLR1 but without using the ‘cross-blocking’ single-domain antibody fragment as disclosed herein. All antibody fragments of the invention specifically exemplified in the experimental part compete with each other for binding to human FOLR1 such that the reduction of the binding of the other antibody fragment is at least 95% (see example 4c), strongly suggesting that all these antibody fragments bind an overlapping epitope on human FOLR1. Within the context of the invention, the word “molecule” when used as a “molecule” that can interfere with the binding of the antibody fragment of the invention may be folic acid or folate or a folate derivative which is the natural ligand of human FOLR1. As natural concentrations of folic acid in the serum are high and are even increased by the uptake of certain nutrients or supplements, antibody-fragments should not compete with folic acid for binding to FOLR1 (Bailey et al. (2010) Am J Clin Nutr 92(2):383-389). In an embodiment, the antibody fragment of the invention does not compete with folic acid for binding to FOLR1. As a result, the antibody fragment of the invention is also expected not to interfere with the natural function of this receptor. The natural function of this receptor is the uptake of folic acid into the cell. It means that in an embodiment, the antibody fragment of the invention does not compete with the natural ligand of human FOLR1 and therefore is not inhibited to bind to human FOLR1-expressing cells in vitro or in an in vivo or ex vivo setting. All antibody fragments specifically exemplified in the experimental part do not compete with the natural ligand of human FOLR1 (see example 4b, FIG. 3).

An antibody fragment, such as a VHH or functional fragments thereof, as disclosed herein is said to show ‘cross-reactivity’ for two different target proteins of interest if it is specific for (as defined herein) both of these different target proteins of interest.

Below we describe several structural features of the antibody fragment of the invention. The antibody fragment of the invention may be characterized by the presence of at least one, at least two, at least three or all of these structural features: first, second, third and fourth structural features.

### First Structural Feature of the Antibody Fragment

A first structural feature is that the antibody fragment of the invention contacts or binds or specifically binds to a region of human FOLR1 comprised within amino acid 25 to 233 of SEQ ID NO:1. SEQ ID NO:1 is the human amino acid sequence of human FOLR1. Amino acid 25 to 233 (R25-M233) of SEQ ID NO:1 is defined as the extracellular domain (without signal peptide M1-T24 and GPI anchor S234 and pro-peptide G235-S257) of human FOLR1 (UniProt Knowledgebase: entry P15328). The region within amino acid 25 to 233 of SEQ ID NO:1 specifically bound or targeted by the antibody fragment of the invention may be a linear region (i.e., linear epitope or sequential epitope) within said primary amino acid sequence. Alternatively said region may not be linear and may correspond to a conformational epitope. Usually a linear epitope comprises a linear sequence of amino acids that has a length of 5 to 30 amino acids, that is to say that it may have a length of 5, 6, 7, 8, 9, 10, 11, 12, 13, 14, 15, 16, 17, 18, 19, 20, 21, 22, 23, 24, 25, 26, 27, 28, 29 or 30 amino acids. Usually a conformational epitope is characterized by a number of non-consecutive amino acids within amino acid 25 to 233 of SEQ ID NO:1 that come together in the three-dimensional tertiary structure of the protein and that are contacted by the antibody fragment.

In the following paragraph dedicated to the second structural feature of the antibody fragment, linear epitopes and conformational epitope of the antibody fragment are defined.

### Second Structural Feature of the Antibody Fragment

The antibody fragment of the invention that specifically binds an epitope of human folate receptor alpha (FOLR1), but does neither bind murine FOLR1 nor human folate receptor beta (FOLR2) nor human folate receptor gamma (FOLR3) may alternatively or in combination also be further defined by a second structural feature.

The antibody fragment of the invention that specifically binds an epitope of human folate receptor alpha (FOLR1), but does neither bind human folate receptor beta (FOLR2) nor human folate receptor gamma (FOLR3) may alternatively or in combination also be further defined by a second structural feature.

A second structural feature is that the antibody fragment of the invention contacts or binds or specifically binds to a number of amino acids within amino acid 25 to 233 of SEQ ID NO:1. These specific amino acids within amino acid 25 to 233 of SEQ ID NO:1 are further defined below.

In an embodiment, the antibody fragment of the invention contacts or binds or specifically binds to at least one of amino acid C89, G90, E91, M92, A93, P94, E140, Q141, W142, W143, E144, D145, C146, R147, T148, S149, Y150, Q176, P177, F178, H179, F180, Y181, F182, P183 and/or T184 of SEQ ID NO:1. Each combination of 2, 3, 4, 5, 6,7, 8, 9, 10, 11, 12, 13, 14, 15, 16, 17, 18, 19, 20, 21, 22, 23, 24, 25 or 26 amino acids from the 26 amino acids identified is therefore encompassed to be contacted by the antibody fragment of the invention.

In an embodiment, the linear stretch of amino acids 89-94 of SEQ ID NO:1 defines a first linear region of hFOLR1, which is contacted, bound or specifically bound by the antibody fragment. Not each amino acid within this stretch or region may be contacted, bound or specifically bound by the antibody fragment. In an embodiment, 1, 2, 3, 4, 5 or 6 amino acids of this linear stretch or region is contacted, bound or specifically bound by the antibody fragment. In an embodiment, this first linear stretch or region is an epitope of the antibody fragment.

In an embodiment, the linear stretch of amino acids 140-150 of SEQ ID NO:1 defines a second linear region of hFOLR1, which is contacted, bound or specifically bound by the antibody fragment. Not each amino acid within this stretch or region needs to be contacted, bound or specifically bound by the antibody fragment. In an embodiment, 1, 2, 3, 4, 5, 6, 7, 8, 9, 10 or 11 amino acids of this linear stretch or region is contacted, bound or specifically bound by the antibody fragment. In an embodiment, this second linear stretch or region is a second epitope of the antibody fragment.

In an embodiment, the linear stretch of amino acids 176-184 of SEQ ID NO:1 defines a third linear region of hFOLR1, which is contacted, bound or specifically bound by the antibody fragment. Not each amino acid within this stretch or region needs to be contacted, bound or specifically bound by the antibody fragment. In an embodiment, 1, 2, 3, 4, 5, 6, 7, 8 or 9 amino acids of this linear stretch or region is contacted, bound or specifically bound by the antibody fragment. In an embodiment, this third linear stretch or region is a third epitope of the antibody fragment.

In an embodiment, each of the first, second and third linear stretches or regions defined above is contacted, bound or specifically bound by the antibody fragment. The combination of these three stretches defines the conformational epitope of the antibody fragment. Not each amino acid within each of these stretches or regions may be contacted, bound or specifically bound by the antibody fragment. In an embodiment, 1, 2, 3, 4, 5 or 6 amino acids (or more depending on the length of each stretch) of each of the linear stretches or regions is contacted, bound or specifically bound by the antibody fragment.

In an embodiment, there is provided an antibody fragment that specifically binds an epitope of human folate receptor alpha (FOLR1), but does neither bind murine FOLR1 nor human folate receptor beta (FOLR2) nor human folate receptor gamma (FOLR3), wherein the epitope comprises the linear amino acid stretch or region 89-94, 140-150 and/or 176-184 of SEQ ID NO:1.

In an embodiment, there is provided an antibody fragment that specifically binds an epitope of human folate receptor alpha (FOLR1), but does neither bind human folate receptor beta (FOLR2) nor human folate receptor gamma (FOLR3), wherein the epitope comprises the linear amino acid stretch or region 89-94, 140-150 and/or 176-184 of SEQ ID NO:1.

In an embodiment, there is provided an antibody fragment, that specifically binds an epitope of human folate receptor alpha (FOLR1), but does neither bind murine FOLR1 nor human folate receptor beta (FOLR2) nor human folate receptor gamma (FOLR3), wherein the epitope comprises the combination of linear amino acid stretches of regions 89-94, 140-150 and 176-184 of SEQ ID NO:1.

In an embodiment, there is provided an antibody fragment, that specifically binds an epitope of human folate receptor alpha (FOLR1), but does neither bind human folate receptor beta (FOLR2) nor human folate receptor gamma (FOLR3), wherein the epitope comprises the combination of linear amino acid stretches of regions 89-94, 140-150 and 176-184 of SEQ ID NO:1.

In an embodiment, the antibody fragment of the invention contacts or binds or specifically binds to the following amino acids of SEQ ID NO:1:
at least one, or at least two, or at least three amino acids selected from C89, G90, E91, M92, A93, or P94, and/or
at least one, or at least two, or at least three amino acids selected from E140, Q141, W142, W143, E144, D145, C146, R147, T148, S149, or Y150, and/or at least one, or at least two, or at least three amino acids selected from Q176, P177, F178, H179, F180, Y181, F182, P183, or T184.

In another embodiment, the antibody fragment of the invention contacts or binds or specifically binds to the following amino acids of SEQ ID NO:1:
at least one, or at least two, or at least three, or at least four, or at least five or all six amino acids selected from C89, G90, E91, M92, A93, or P94, and
at least one, or at least two, or at least three, or at least four, or at least five, or at least six or at least seven, or at least eight or at least nine or at least ten or all eleven amino acids selected from E140, Q141, W142, W143, E144, D145, C146, R147, T148, S149, or Y150, and
at least one, or at least two, or at least three, or at least four, or at least five, or at least six, or at least seven, or at least eight, or all nine amino acids selected from Q176, P177, F178, H179, F180, Y181, F182, P183, or T184.

In another embodiment, the antibody fragment contacts or binds or specifically binds to the following amino acids of SEQ ID NO:1:
1) at least two, or at least three amino acids selected from C89, G90, E91, M92, A93, or P94, and/or
2) at least two, or at least three amino acids selected from E140, Q141, W142, W143, E144, D145, C146, R147, T148, S149, or Y150, and/or
3) at least two, or at least three amino acids selected from Q176, P177, F178, H179, F180, Y181, F182, P183, or T184.

In another embodiment, the antibody fragment contacts or binds or specifically binds to the following amino acids of SEQ ID NO:1:
1) at least two, or at least three amino acids selected from C89, G90, E91, M92, A93, or P94, and
2) at least two, or at least three amino acids selected from E140, Q141, W142, W143, E144, D145, C146, R147, T148, S149, or Y150, and
3) at least two, or at least three amino acids selected from Q176, P177, F178, H179, F180, Y181, F182, P183, or T184.

In another embodiment, the antibody fragment contacts or binds or specifically binds to the following amino acids of SEQ ID NO:1:
1) at least three amino acids selected from C89, G90, E91, M92, A93, or P94, and/or
2) at least three amino acids selected from E140, Q141, W142, W143, E144, D145, C146, R147, T148, S149, or Y150, and/or
3) at least three amino acids selected from Q176, P177, F178, H179, F180, Y181, F182, P183, or T184.

In another embodiment, the antibody fragment contacts or binds or specifically binds to the following amino acids of SEQ ID NO:1:
1) at least three amino acids selected from C89, G90, E91, M92, A93, or P94, and
2) at least three amino acids selected from E140, Q141, W142, W143, E144, D145, C146, R147, T148, S149, or Y150, and
3) or at least three amino acids selected from Q176, P177, F178, H179, F180, Y181, F182, P183, or T184.

In an embodiment, the antibody fragment of the invention contacts or binds or specifically binds to the following amino acids of SEQ ID NO:1: C89, G90, E91, M92, A93 and/or P94.

In an embodiment, the antibody fragment of the invention contacts or binds or specifically binds to the following amino acids of SEQ ID NO:1: E140, Q141, W142, W143, E144, D145, C146, R147, T148, S149 and/or Y150.

In an embodiment, the antibody fragment of the invention contacts or binds or specifically binds to the following amino acids of SEQ ID NO:1: Q176, P177, F178, H179, F180, Y181, F182, P183 and/or T184.

In an embodiment, the following amino acids of amino acids 25 to 233 of SEQ ID NO:1 are contacted, bound or specifically bound by the antibody fragment: C89, G90, E91, M92, A93, P94, E140, Q141, W142, W143, E144, D145, C146, R147, T148, S149, Y150, Q176, P177, F178, H179, F180, Y181, F182, P183 and T184.

### Third Structural Feature of the Antibody Fragment

The antibody fragment of the invention that specifically binds an epitope of human folate receptor alpha (FOLR1), but does neither bind murine FOLR1 nor human folate receptor beta (FOLR2) nor human folate receptor gamma (FOLR3) may alternatively or in combination also be further defined by a third structural feature.

The antibody fragment of the invention that specifically binds an epitope of human folate receptor alpha (FOLR1), but does neither bind human folate receptor beta (FOLR2) nor human folate receptor gamma (FOLR3) may alternatively or in combination also be further defined by a third structural feature.

A third structural feature relates to the (full length) amino acid sequence representing a way of defining the family of antibody fragment of the invention. The present invention discloses a family of structurally closely related antibody fragments represented by an amino acid sequence comprising, consisting of or essentially consisting of SEQ ID NO: 8 or 9. Antibody fragment A1 is represented by SEQ ID NO:8 and antibody fragment A2 by SEQ ID NO:9 (see table 2 below).

Each of SEQ ID NO:2, 4 and 5 is a part of antibody fragment A1 which is conserved amongst the family of the antibody fragment of the invention (see table 2 below).

Each of SEQ ID NO:3, 6 and 7 is a part of antibody fragment A2 which is conserved amongst the family of the antibody fragment of the invention (see table 2 below).

Antibody fragments A1 and A2 both contact, bind or specifically bind each of the linear stretches or regions of amino acids of SEQ ID NO:1 as defined earlier herein (i.e. linear amino acid stretch or region 89-94, 140-150 and/or 176-184 of SEQ ID NO:1). Moreover, both A1 and A2 have for conformational epitope the combination of stretches or regions of amino acids of SEQ ID NO:1 as defined earlier herein (i.e., linear amino acid stretches or regions 89-94, 140-150 and 176-184 of SEQ ID NO:1). A1 and A2 are two members of a family of antibody fragments. This family of antibody fragments shares at least one of these linear epitopes and/or this conformational epitope. This family of antibody fragment has exceptional kinetic characteristics (see example 4) and/or exceptional tissue distribution when used as radiolabelled antibody fragment (see examples 6, 7 and 8).

TABLE 2

conserved part of antibody fragments A1 or A2

| | |
|---|---|
| A1 | Amino acid sequence |
| SEQ ID NO:2 | NHMSWYRQAPGKQRELAAIITSDGNTNYPDSVKGRFTI SRDNAKNTVYLQMNSLKPEDTAVYYCNTFGRSVAGFY DYW |
| SEQ ID NO:4 | NHMSWYRQAPGKQRELAAIITSDGNTNYPD |
| SEQ ID NO:5 | NTFGRSVAGFYDYW |
| SEQ ID NO:8 | DVQLVESGGGLVQPGGSLRLSCAASESIFSRNHMSWY RQAPGKQRELAAIITSDGNTNYPDSVKGRFTISRDNAKN TVYLQMNSLKPEDTAVYYCNTFGRSVAGFYDYWGQGT QVTVSS |
| SEQ ID NO:3 | DLAWYRRAPGKQRELAAIITSGGSTNYPDSVKGRFTISR DNAKNTVYLEMNSLKPEDTAVYYCNALRPWPQFSDYW |
| SEQ ID NO:6 | DLAWYRRAPGKQRELAAIITSGGSTNYPD |
| SEQ ID NO:7 | NALRPWPQFSDYW |
| SEQ ID NO:9 | DVQLVESGGGLVQAGGSLRLSCAADEMKFSNYDLAWY RRAPGKQRELAAIITSGGSTNYPDSVKGRFTISRDNAKN TVYLEMNSLKPEDTAVYYCNALRPWPQFSDYWGQGTQ VTVSS |

This third structural feature of the antibody fragment of the invention may be defined in several ways:

In a first embodiment of this third structural feature, the antibody fragment of the invention is represented by an amino acid sequence that comprises or essentially consists of an amino acid sequence having at least 60% sequence identity (or similarity) with at least one of SEQ ID NO:2, 3, 4, 5, 6, 7, 8 or 9 or a portion thereof. In an embodiment, the sequence identity (or similarity) with at least of one of these sequences is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a second embodiment of this third structural feature, the antibody fragment of the invention is represented by an amino acid sequence that comprises or essentially consists of an amino acid sequence having at least 60% sequence identity (or similarity) with at least one of SEQ ID NO:2, 3, 4, 5, 6, 7, 8 or 9 or a portion thereof and has a length which is ranged from the exact length of SEQ ID NO: 2, 3, 4, 5, 6, 7, 8 or 9 or 1, 2, 3, 4, 5, 6, 7, 8, 9, 10, 15, 20, 25, 30, 35, 40, 45, 50, 55, 60 amino acids longer than the exact length of SEQ ID NO: 2, 3, 4, 5, 6, 7, 8 or 9. For example a tag such as a His tag may be added to the antibody fragment of the invention. Usually His tag comprises 4, 5, 6, 7, 8, 9, 10 histidines. Alternative tag may be a Hemaglutinin tag (HA-tag): YPYDVPDYA (SEQ ID NO: 79); YPYDVPDYGS (SEQ ID NO: 80) or a cysteine tag (Cys tag). A cysteine tag is a tag that comprises one or several cysteine. Non-limiting examples of cysteine tags are C; GGC; SPSTPPTPSPSTPPC (SEQ ID NO: 81)

The way identity and similarity are assessed is explained in detail in the part dedicated to definition as the end of the description. Usually when identity is defined by reference to a SEQ ID NO, said identity is assessed over the whole SEQ ID NO. However, it is also encompassed by the invention that identity (or similarity) is assessed over a portion (or a fragment) of said sequence. Within this context, a portion may mean at least 50%, 60%, 70%, 80%, 90%, 95% of the length of the SEQ ID NO. The length of the sequence encompassed may still be longer than the length of the SEQ ID NO used to assess the identity (or similarity) (i.e. length being at least 50% of the length of the SEQ ID NO, 60%, 70%, 80%, 90%, the same as the one of the SEQ ID NO even though the identity(or similarity) is assessed over a portion of this SEQ ID NO, or the length being 1, 5, 10, 15, 20, 25, 30, 35, 40, 45, 50, 55, 60 amino acids longer than the exact length of SEQ ID NO: 2, 3, 4, 5, 6, 7, 8 or 9).

In a third embodiment of this third structural feature, the length of the antibody fragment is from 110 to 130 amino acids or 110, 111, 112, 113, 114, 115, 116, 117, 118, 119, 120, 121, 122, 123, 124, 125, 126, 127, 128, 129 or 130 amino acids. This length does not include the length of a tag, such as a His tag that may be added to the sequence of the antibody fragment.

In a fourth embodiment of this third structural feature, the antibody fragment of the invention is represented by an amino acid sequence that comprises or essentially consists of an amino acid sequence having at least 60% sequence identity (or similarity) with at least one of SEQ ID NO:2, 3, 4, 5, 6, 7, 8 or 9 or a portion thereof and the length of the antibody fragment is from 80 to 150 amino acids or 90 to 140 or 100 to 130 or 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) with at least of one of these sequences is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a fifth embodiment of this third structural feature, the antibody fragment of the invention is represented by an amino acid sequence that comprises or essentially consists of an amino acid sequence having at least 60% sequence identity (or similarity) with SEQ ID NO:2 or a portion thereof. In an embodiment, the antibody fragment is represented by an amino acid sequence that comprises or essentially consists of an amino acid sequence having at least 60% sequence identity (or similarity) with SEQ ID NO:2 or a portion thereof and has a length which is ranged from 78 to 130 amino acids or 78 to 130 or 90 to 120 or 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a sixth embodiment of this third structural feature, the antibody fragment of the invention is represented by an amino acid sequence that comprises or essentially consists of an amino acid sequence having at least 60% sequence identity (or similarity) with SEQ ID NO:4 and/or 5 or a portion thereof. In an embodiment, the antibody fragment is represented by an amino acid sequence that comprises or essentially consists of an amino acid sequence having at least 60% sequence identity (or similarity) with any of SEQ ID NO:4 and/or 5 or a portion thereof and has a length which is ranged from 14 to 130 amino acids or 20 to 120 or 30 to 110 or 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72. 73. 74. 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a seventh embodiment of this third structural feature, the antibody fragment of the invention is represented by an amino acid sequence that comprises or essentially consists of an amino acid sequence having at least 60% sequence identity (or similarity) with SEQ ID NO:3 or a portion thereof. In an embodiment, the antibody fragment is represented by an amino acid sequence that comprises or essentially consists of an amino acid sequence having at least 60% sequence identity (or similarity) with any of SEQ ID NO:3 or a portion thereof and has a length which is ranged from 76 to 130 amino acids or 76 to 120 or 90 to 110 or 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In an eighth embodiment of this third structural feature, the antibody fragment of the invention is represented by an amino acid sequence that comprises or essentially consists of an amino acid sequence having at least 60% sequence identity (or similarity) with SEQ ID NO:6 and/or 7 or a portion thereof. In an embodiment, the antibody fragment is represented by an amino acid sequence that comprises or essentially consists of an amino acid sequence having at least 60% sequence identity (or similarity) with SEQ ID NO:6 and/or 7 or a portion thereof and has a length which is ranged from 13 to 150 amino acids or 13 to 140 or 30 to 130 or 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72. 73. 74. 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a ninth embodiment of this third structural feature, the antibody fragment of the invention is represented by an amino acid sequence that comprises or essentially consists of an amino acid sequence having at least 60% sequence identity (or similarity) with SEQ ID NO:8 and/or 9 or a portion thereof. In an embodiment, the antibody fragment is represented by an amino acid sequence that comprises or essentially consists of an amino acid sequence having at least 60% sequence identity (or similarity) with SEQ ID NO:8 and/or 9 or a portion thereof and has a length which is ranged from 110 to 130 amino acids or 100 to 130 or 110 to 120 or 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a tenth embodiment of this third structural feature, the antibody fragment, preferably a VHH or a functional fragment thereof is represented by an amino acid sequence that comprises any of SEQ ID NO:2, 3, 4, 5, 6 and/or 7. In an embodiment, it has a length which is ranged from 13 to 130 amino acids or 20 to 120 or 30 to 121 amino acids. More preferably it is represented by an amino acid sequence that comprises SEQ ID NO:8 or 9. Even more preferably, it has a length of 118, 119, 120, 121, 122, 123, 124, 125, 126, 127, 128, 129 or 130 amino acids.

In a further embodiment, the antibody fragment of the invention is according to the ninth embodiment of this third structural feature and contacts or binds or specifically binds to the following amino acids of SEQ ID NO:1:
at least one, or at least two, or at least three amino acids selected from C89, G90, E91, M92, A93, or P94, and/or
at least one, or at least two, or at least three amino acids selected from E140, Q141, W142, W143, E144, D145, C146, R147, T148, S149, or Y150, and/or
at least one, or at least two, or at least three amino acids selected from Q176, P177, F178, H179, F180, Y181, F182, P183, or T184.

In a further embodiment, the antibody fragment of the invention is according to the tenth embodiment of this third structural feature and contacts or binds or specifically binds to the following amino acids of SEQ ID NO:1:
at least one, or at least two, or at least three amino acids selected from C89, G90, E91, M92, A93, or P94, and/or
at least one, or at least two, or at least three amino acids selected from E140, Q141, W142, W143, E144, D145, C146, R147, T148, S149, or Y150, and/or
at least one, or at least two, or at least three amino acids selected from Q176, P177, F178, H179, F180, Y181, F182, P183, or T184.

In an embodiment, an antibody fragment specifically binds an epitope of human folate receptor alpha (FOLR1), but does neither bind murine FOLR1 nor human folate receptor beta (FOLR2) nor human folate receptor gamma (FOLR3), wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence.

In an embodiment, an antibody fragment specifically binds an epitope of human folate receptor alpha (FOLR1), but does neither bind human folate receptor beta (FOLR2) nor human folate receptor gamma (FOLR3), wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence. In an embodiment, the sequence identity (or similarity) is at least 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, an antibody fragment specifically binds an epitope of human folate receptor alpha (FOLR1), but does neither bind murine FOLR1 nor human folate receptor beta (FOLR2) nor human folate receptor gamma (FOLR3), wherein the epitope comprises the linear amino acid stretch or region 89-94, 140-150 and/or 176-184 of SEQ ID NO:1 and wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence.

In an embodiment, an antibody fragment specifically binds an epitope of human folate receptor alpha (FOLR1), but does neither bind human folate receptor beta (FOLR2) nor human folate receptor gamma (FOLR3), wherein the epitope comprises the linear amino acid stretch or region 89-94, 140-150 and/or 176-184 of SEQ ID NO:1 and wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence.

In an embodiment, the sequence identity (or similarity) is at least 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, an antibody fragment specifically binds an epitope of human folate receptor alpha (FOLR1), but does neither bind murine FOLR1 nor human folate receptor beta (FOLR2) nor human folate receptor gamma (FOLR3), wherein the epitope comprises the combination of linear amino acid stretches or regions 89-94, 140-150 and 176-184 of SEQ ID NO:1, and wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence.

In an embodiment, an antibody fragment specifically binds an epitope of human folate receptor alpha (FOLR1), but does neither bind human folate receptor beta (FOLR2) nor human folate receptor gamma (FOLR3), wherein the epitope comprises the combination of linear amino acid stretches or regions 89-94, 140-150 and 176-184 of SEQ ID NO:1, and wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence.

In an embodiment, the sequence identity (or similarity) is at least 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

### Fourth Structural Feature

The antibody fragment of the invention that specifically binds an epitope of human folate receptor alpha (FOLR1), but does neither bind murine FOLR1 nor human folate receptor beta (FOLR2) nor human folate receptor gamma (FOLR3) may also be alternatively or in combination further defined by a fourth structural feature.

The antibody fragment of the invention that specifically binds an epitope of human folate receptor alpha (FOLR1), but does neither bind human folate receptor beta (FOLR2) nor human folate receptor gamma (FOLR3) may also be alternatively or in combination further defined by a fourth structural feature.

In a fourth structural feature, the antibody fragment of the invention, preferably the VHH’s as disclosed herein is represented by an amino acid sequence that comprises at least one combination of CDR sequences chosen from the group comprising:
a CDR1 region having SEQ ID NO: 10, a CDR2 region having has SEQ ID NO: 11, and a CDR3 region having SEQ ID NO: 12, and/or
a CDR1 region having SEQ ID NO: 17 a CDR2 region having has SEQ ID NO: 18, and a CDR3 region having SEQ ID NO: 19 (see table 3).

Thus, in particular embodiments, the present invention provides heavy chain variable domains comprising the heavy chain antibodies with the (general) structure or which is derived therefrom:

FR1 - CDR1 - FR2 - CDR2 - FR3 - CDR3 - FR4

in which FR1 to FR4 refer to framework regions 1 to 4, respectively, and in which CDR1 to CDR3 refer to the complementarity determining regions 1 to 3, respectively, and are as further defined herein. SEQ ID NO’s: 8 and 9 (see table 3) give the amino acid sequences of heavy chain variable domains that have been raised against human FOLR1.

TABLE 3

CDR and FR of antibody fragments A1 and A2 (using the IMGT nomenclature as defined later on herein in the general part of the description dedicated to the general definition of the invention)
A1 (SEQ ID NO: 8)

| CDR of A1 | |
|---|---|
| CDR1 (SEQ ID NO: 10) | ESIFSRNH |
| CDR2 (SEQ ID NO: 11) | ITSDGNT |
| CDR3 (SEQ ID NO: 12) | NTFGRSVAGFYDY |
| FR of A1 | |
| FR1 (SEQ ID NO: 13) | DVQLVESGGGLVQPGGSLRLSCAAS |
| FR2 (SEQ ID NO: 14) | MSWYRQAPGKQRELAAI |
| FR3 (SEQ ID NO: 15) | NYPDSVKGRFTISRDNAKNTVYLQMNSLKPEDTAVYYC |
| FR4 (SEQ ID NO: 16) | WGQGTQVTVSS |
| A2 (SEQ ID NO:9) | |
| CDR of A2 | |
| CDR1 (SEQ ID NO: 17) | EMKFSNYD |
| CDR2 (SEQ ID NO: 18) | ITSGGST |
| CDR3 (SEQ ID NO: 19) | NALRPWPQFSDY |
| FR of A2 | |
| FR1 (SEQ ID NO: 20) | DVQLVESGGGLVQAGGSLRLSCAAD |
| FR2 (SEQ ID NO: 21) | LAWYRRAPGKQRELAAI |
| FR3 (SEQ ID NO: 22) | NYPDSVKGRFTISRDNAKNTVYLEMNSLKPEDTAVYYC |
| FR4 (SEQ ID NO: 23) | WGQGTQVTVSS |

It should be noted that the invention is not limited as to the origin of the antibody fragment, preferably VHH or fragments thereof disclosed herein (or of the nucleotide sequences to express these), nor as to the way that the antibody fragment, preferably VHH or fragments thereof or nucleotide sequences disclosed herein are (or have been) generated or obtained. Thus, the antibody fragment, preferably VHH or fragment thereof disclosed herein may be naturally occurring amino acid sequences (from any suitable species) or synthetic or semi-synthetic amino acid sequences. Methods for isolating antibody fragment and methods of producing antibody fragment as well as nucleic acid molecule encoding the antibody fragment, construct comprising these nucleic acid molecule and cells comprising these constructs are disclosed in detail in the definition part at the end of the description.

In a specific but non-limiting aspect of the invention, the amino acid sequence of the antibody fragment is a naturally occurring immunoglobulin sequence (from any suitable species) or a synthetic or semi-synthetic immunoglobulin sequence, including but not limited to “humanized” immunoglobulin sequences (such as partially or fully humanized mouse or rabbit immunoglobulin sequences, and in particular partially or fully humanized VHH sequences), “camelized” immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Also, an antibody fragment, preferably a VHH or fragments thereof as disclosed herein may be suitably humanized, as further described herein, so as to provide one or more further (partially or fully) humanized amino acid sequences of the invention.

In an embodiment, the antibody fragment of the invention is derived from the antibody fragments described above using CDR grafting. Preferred antibody fragments comprise a:
CDR1 region having SEQ ID NO: 10, a CDR2 region having has SEQ ID NO: 18, and a CDR3 region having SEQ ID NO: 12,
CDR1 region having SEQ ID NO: 10, a CDR2 region having has SEQ ID NO: 18, and a CDR3 region having SEQ ID NO: 19,
CDR1 region having SEQ ID NO: 10, a CDR2 region having has SEQ ID NO: 11, and a CDR3 region having SEQ ID NO: 19,
CDR1 region having SEQ ID NO: 17 a CDR2 region having has SEQ ID NO: 11, and a CDR3 region having SEQ ID NO: 19,
CDR1 region having SEQ ID NO: 17 a CDR2 region having has SEQ ID NO: 11, and a CDR3 region having SEQ ID NO: 12, or
CDR1 region having SEQ ID NO: 17 a CDR2 region having has SEQ ID NO: 18, and a CDR3 region having SEQ ID NO: 12.
Each of these antibody fragment may have the FR of A1 or of A2 as identified in table 3. However, distinct FR may be present.

In another embodiment, the antibody fragment of the invention is derived from the antibody fragments described above using CDR grafting. Preferred antibody fragments comprise a:
CDR1 region having SEQ ID NO: 10 and a CDR3 region having SEQ ID NO: 12,
CDR1 region having SEQ ID NO: 10 and a CDR3 region having SEQ ID NO: 19,
CDR1 region having SEQ ID NO: 17 and a CDR3 region having SEQ ID NO: 19,
CDR1 region having SEQ ID NO: 17 and a CDR3 region having SEQ ID NO: 12,

Each of these antibody fragment may comprise a CDR2 region from another VHH and may have the FR of A1 or of A2 as identified in table 3. However, distinct FR may be present.

In another embodiment, the antibody fragment of the invention is derived from the antibody fragments described above using CDR grafting. Preferred antibody fragments comprise a:
CDR1 region having SEQ ID NO: 10 and a CDR2 region having SEQ ID NO: 11,
CDR1 region having SEQ ID NO: 10 and a CDR2 region having SEQ ID NO: 18,
CDR1 region having SEQ ID NO: 17 and a CDR2 region having SEQ ID NO: 18,
CDR1 region having SEQ ID NO: 17 and a CDR2 region having SEQ ID NO: 11,

Each of these antibody fragment may comprise a CDR3 region from another VHH and may have the FR of A1 or of A2 as identified in table 3. However, distinct FR may be present.

In another embodiment, the antibody fragment of the invention is derived from the antibody fragments described above using CDR grafting. Preferred antibody fragments comprise a:
CDR2 region having SEQ ID NO: 11 and a CDR3 region having SEQ ID NO: 12,
CDR2 region having SEQ ID NO: 11 and a CDR3 region having SEQ ID NO: 19,
CDR2 region having SEQ ID NO: 18 and a CDR3 region having SEQ ID NO: 19,
CDR2 region having SEQ ID NO: 18 and a CDR3 region having SEQ ID NO: 12,

Each of these antibody fragment may comprise a CDR1 region from another VHH and may have the FR of A1 or of A2 as identified in table 3. However, distinct FR may be present.

In another embodiment, the antibody fragment of the invention is derived from the antibody fragments described above using CDR grafting. Preferred antibody fragments comprise a:

CDR1 region having SEQ ID NO: 10 or 17 and may comprise a CDR2 and CDR3 region from another VHH and may have the FR of A1 or of A2 as identified in table 3. However, distinct FR may be present.

In another embodiment, the antibody fragment of the invention is derived from the antibody fragments described above using CDR grafting. Preferred antibody fragments comprise a:

CDR2 region having SEQ ID NO: 11 or 18 and may comprise a CDR1 and CDR3 region from another VHH and may have the FR of A1 or of A2 as identified in table 3. However, distinct FR may be present.

In another embodiment, the antibody fragment of the invention is derived from the antibody fragments described above using CDR grafting. Preferred antibody fragments comprise a:

CDR3 region having SEQ ID NO: 12 or 19 and may comprise a CDR1 and CDR2 region from another VHH and may have the FR of A1 or of A2 as identified in table 3. However, distinct FR may be present.

Similarly, when an amino acid sequence comprises a synthetic or semi-synthetic sequence (such as a partially humanized sequence), said sequence may optionally be further suitably humanized, again as described herein, so as to provide one or more further (partially or fully) humanized amino acid sequences as disclosed herein. At the end of the description, a more detailed definition of “agonist” “antagonist”, “variants of antibody fragment”, “posttranslational structural characterization of antibody fragment” is provided.

In particular, humanized antibody fragment, preferably VHH may be represented by amino acid sequences in which at least one amino acid residue is present (and in particular, in at least one of the framework residues) that is and/or that corresponds to a humanizing substitution. In addition, or alternatively, other potentially useful humanizing substitutions can be ascertained by comparing the sequence of the framework regions of a naturally occurring VHH sequence with the corresponding framework sequence of one or more closely related human V_{H} sequences, after which one or more of the potentially useful humanizing substitutions (or combinations thereof) thus determined can be introduced into said VHH sequence (in any manner known per se, as further described herein) and the resulting humanized VHH sequences or functional fragments thereof can be tested for affinity for the target, for stability, for ease and level of expression, and/or for other desired properties. In this way, by means of a limited degree of trial and error, other suitable humanizing substitutions (or suitable combinations thereof) can be determined by the skilled.

In an embodiment, the antibody fragment, preferably a VHH of the invention (or a fragment thereof) is represented by a first, second, third and/or fourth structural feature as identified herein. Alternatively, or in combination with said structural feature, said antibody fragment, preferably a VHH of the invention (or a fragment thereof) is characterized by a functional feature which is to specifically bind human folate receptor alpha (FOLR1), but (preferably not murine FOLR1), not human folate receptor beta (FOLR2) and not human folate receptor gamma (FOLR3). In an embodiment, the antibody fragment, preferably a VHH of the invention (or a fragment thereof) is characterized by a functional feature which is to specifically bind human folate receptor alpha (FOLR1), but (preferably not murine FOLR1), not human folate receptor beta (FOLR2) and not human folate receptor gamma (FOLR3). An antibody fragment, VHH or fragment of a VHH of the invention should therefore fulfil at least one of the structural features and/or functional features.

### Additional Antibody Fragment

In a further aspect there is provided additional antibody fragments having similar structural and/or functional characteristics as the ones disclosed before.

These additional antibody fragment such as a single-domain antibody fragment, preferably a VHH or fragment thereof may be characterized by a functional feature and/or by a structural feature. Examples of structural features are sequence related and examples of functional features are related to an activity of said antibody fragment. The wording present earlier herein and related to the definition of an “antibody fragment”, of “its activity”, of “its binding”, “cross-binding” (i.e., competing with), “affinity”, “avidity”, and/or “specificity” also applied to these additional antibody fragments.

In an embodiment, this antibody fragment specifically binds human folate receptor alpha (FOLR1, which is represented by SEQ ID NO:1), but does neither bind murine FOLR1 nor human folate receptor beta (FOLR2) nor human folate gamma receptor (FOLR3) (or an antibody fragment specifically binding human FOLR1, but neither binding human FOLR2 nor human FOLR3) and fulfils at least one of the following:
a. the binding is comprised within amino acid 25 to 233 of SEQ ID NO:1,
b. at least amino acid C89, G90, E91, M92, A93, P94, E140, Q141, W142, W143, E144, D145, C146, R147, T148, S149, Y150, Q176, P177, F178, H179, F180, Y181, F182, P183 and/or T184 of SEQ ID NO:1 interacts with said antibody fragment,
c. said antibody fragment is represented by an amino acid sequence that comprises an amino acid sequence having at least 60% sequence identity (or similarity) with at least one of SEQ ID NO:24, 32, 40, 61-69 or a portion thereof.

In an embodiment, the epitope of the additional antibody fragment comprises the linear amino acid stretch or region 89-94, 140-150 and/or 176-184 of SEQ ID NO:1.

In an embodiment, the epitope of the additional antibody fragment comprises the combination of linear amino acid stretches or regions 89-94, 140-150 and 176-184 of SEQ ID NO:1.

In an embodiment, this additional antibody fragment specifically binds human folate receptor alpha (FOLR1, which is represented by SEQ ID NO:1), but does neither bind murine FOLR1 nor human folate receptor beta (FOLR2) nor human folate gamma receptor (FOLR3) (or an antibody fragment specifically binding human FOLR1, but neither binding human FOLR2 nor human FOLR3) and fulfils a) and/or b):
a) the epitope is comprised within amino acid 25 to 233 of SEQ ID NO:1 and the antibody fragment specifically binds to the following amino acids of SEQ ID NO:1:
   1) at least one, or at least two, or at least three amino acids selected from C89, G90, E91, M92, A93, or P94, and/or
   2) at least one, or at least two, or at least three amino acids selected from E140, Q141, W142, W143, E144, D145, C146, R147, T148, S149, or Y150, and/or
   3) at least one, or at least two, or at least three amino acids selected from Q176, P177, F178, H179, F180, Y181, F182, P183, or T184,
b) said antibody fragment is represented by an amino acid sequence that comprises an amino acid sequence having at least 60% sequence identity (or similarity) with at least one of SEQ ID NO:24, 32, 40, 61-69 or a portion thereof.

In an embodiment, the sequence identity (or similarity) with at least of one of these sequences is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, this additional antibody fragment specifically binds human folate receptor alpha (FOLR1, which is represented by SEQ ID NO:1), but does neither bind murine FOLR1 nor human folate receptor beta (FOLR2) nor human folate gamma receptor (FOLR3) (or an antibody fragment specifically binding human FOLR1, but neither binding human FOLR2 nor human FOLR3), the epitope is comprised within amino acid 25 to 233 of SEQ ID NO:1 and the antibody fragment specifically binds to the following amino acids of SEQ ID NO:1:
1) at least one, or at least two, or at least three amino acids selected from C89, G90, E91, M92, A93, or P94, and
2) at least one, or at least two, or at least three amino acids selected from E140, Q141, W142, W143, E144, D145, C146, R147, T148, S149, or Y150, and
3) at least one, or at least two, or at least three amino acids selected from Q176, P177, F178, H179, F180, Y181, F182, P183, or T184.

In an embodiment, the epitope of the additional antibody fragment comprises the linear amino acid stretch or region 89-94, 140-150 and/or 176-184 of SEQ ID NO:1 and said additional antibody fragment is represented by an amino acid sequence that comprises an amino acid sequence having at least 60% sequence identity (or similarity) with at least one of SEQ ID NO:24, 32, 40, 61-69 or a portion thereof. In an embodiment, the sequence identity (or similarity) with at least of one of these sequences is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the epitope of the additional antibody fragment comprises the combination of linear amino acid stretches or regions 89-94, 140-150 and 176-184 of SEQ ID NO:1 and said additional antibody fragment is represented by an amino acid sequence that comprises an amino acid sequence having at least 60% sequence identity (or similarity) with at least one of SEQ ID NO:24, 32, 40, 61-69 or a portion thereof. In an embodiment, the sequence identity (or similarity) with at least of one of these sequences is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

This additional antibody fragment could be used the same way as the other ones. All functional definitions provided earlier or later herein also apply to this additional antibody fragment. The first and second structural features of the antibody fragment already defined herein also apply to these additional antibody fragments.

Below we describe several additional structural features of the additional antibody fragment of the invention. The antibody fragment of the invention may be characterized by the presence of at least one, at least two, at least three or all of these structural features: first, second, third and fourth structural features.

### Third Structural Feature of the Additional Antibody Fragment

The additional antibody fragment of the invention that specifically binds an epitope of human folate receptor alpha (FOLR1), but does neither bind murine FOLR1 nor human folate receptor beta (FOLR2) nor human folate receptor gamma (FOLR3) may also alternatively or in combination be further defined by a third structural feature.

The additional antibody fragment of the invention that specifically binds an epitope of human folate receptor alpha (FOLR1), but does neither bind human folate receptor beta (FOLR2) nor human folate receptor gamma (FOLR3) may also alternatively or in combination be further defined by a third structural feature.

A third structural feature relates to the (full length) amino acid sequence representing a way of defining the family of antibody fragment of the invention. The present invention discloses a family of structurally closely related antibody fragments represented by an amino acid sequence comprising, consisting of or essentially consisting of SEQ ID NO: 24 or 32 or 40. Antibody fragment A3 is represented by SEQ ID NO:24, antibody fragment A4 by SEQ ID NO: 32 and antibody fragment A5 by SEQ ID NO:40 (see table 4 below).

Each of SEQ ID NO:61, 62 and 63 is a part of antibody fragment A3 which is conserved amongst the family of the antibody fragment of the invention (see table 4 below) and which is also conserved amongst the antibody fragments described earlier herein.

Each of SEQ ID NO:64, 65 and 66 is a part of antibody fragment A4 which is conserved amongst the family of the antibody fragment of the invention (see table 4 below) and which is also conserved amongst the antibody fragments described earlier herein.

Each of SEQ ID NO:67, 68 and 69 is a part of antibody fragment A5 which is conserved amongst the family of the antibody fragment of the invention (see table 4 below) and which is also conserved amongst the antibody fragments described earlier herein.

In short, SEQ ID NO: 2, 3, 61, 64 and 67 are conserved amongst all the antibodies fragments described herein.

In short, SEQ ID NO: 4, 6, 62, 65 and 68 are conserved amongst all the antibodies fragments described herein.

In short, SEQ ID NO: 5, 7, 63, 66 and 69 are conserved amongst all the antibodies fragments described herein.

This third structural feature of the additional antibody fragment of the invention may be defined in several ways:

In a first embodiment of this third structural feature, the antibody fragment of the invention is represented by an amino acid sequence that comprises or essentially consists of an amino acid sequence having at least 60% sequence identity (or similarity) with at least one of SEQ ID NO: 61, 62, 63, 64, 65, 66, 67, 68, 69, 24, 32 and 40 or a portion thereof. In an embodiment, the sequence identity (or similarity) with at least of one of these sequences is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a second embodiment of this third structural feature, the antibody fragment of the invention is represented by an amino acid sequence that comprises or essentially consists of an amino acid sequence having at least 60% sequence identity (or similarity) with at least one of SEQ ID NO: 61, 62, 63, 64, 65, 66, 67, 68, 69, 24, 32 and 40 or a portion thereof and has a length which is ranged from the exact length of SEQ ID NO: 61, 62, 63, 64, 65, 66, 67, 68, 69, 24, 32 and 40 or 1, 2, 3, 4, 5, 6, 7, 8, 9, 10, 15, 20, 25, 30, 35, 40, 45, 50, 55, 60 amino acids longer than the exact length of SEQ ID NO: 61, 62, 63, 64, 65, 66, 67, 68, 69, 24, 32 and 40. For example a tag such as a His tag may be added to the antibody fragment of the invention. Usually His tag comprises 4, 5, 6, 7, 8, 9, 10 Histidines. Alternative tag may be a Hemaglutinin tag (HA-tag): YPYDVPDYA (SEQ ID NO: 79); YPYDVPDYGS (SEQ ID NO: 80) or a cysteine tag (Cys tag). A cysteine tag is a tag that comprises one or several cysteine. Non-limiting examples of cysteine tags are C; GGC; SPSTPPTPSPSTPPC (SEQ ID NO: 81).

The way identity and similarity are assessed is explained in detail in the part dedicated to definition as the end of the description. Usually when identity is defined by reference to a SEQ ID NO, said identity is assessed over the whole SEQ ID NO. However, it is also encompassed by the invention that identity (or similarity) is assessed over a portion (or a fragment) of said sequence. Within this context, a portion may mean at least 50%, 60%, 70%, 80%, 90%, 95% of the length of the SEQ ID NO. The length of the sequence encompassed may still be longer than the length of the SEQ ID NO used to assess the identity (or similarity) (i.e. length being at least 50% of the length of the SEQ ID NO, 60%, 70%, 80%, 90%, the same as the one of the SEQ ID NO even though the identity (or similarity) is assessed over a portion of this SEQ ID NO, or the length being 1, 5, 10, 15, 20, 25, 30, 35, 40, 45, 50, 55, 60 amino acids longer than the exact length of SEQ ID NO: 61, 62, 63, 64, 65, 66, 67, 68, 69, 24, 32 and 40).

In a third embodiment of this third structural feature, the length of the antibody fragment is from 110 to 130 amino acids or 110, 111, 112, 113, 114, 115, 116, 117, 118, 119, 120, 121, 122, 123, 124, 125, 126, 127, 128, 129 or 130 amino acids. This length does not include the length of a tag, such as a His tag that may be added to the sequence of the antibody fragment.

In a fourth embodiment of this third structural feature, the antibody fragment of the invention is represented by an amino acid sequence that comprises or essentially consists of an amino acid sequence having at least 60% sequence identity (or similarity) with at least one of SEQ ID NO: 61, 62, 63, 64, 65, 66, 67, 68, 69, 24, 32 and 40 or a portion thereof and the length of the antibody fragment is from 80 to 150 amino acids or 90 to 140 or 100 to 130 or 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) with at least of one of these sequences is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a fifth embodiment of this third structural feature, the antibody fragment of the invention is represented by an amino acid sequence that comprises or essentially consists of an amino acid sequence having at least 60% sequence identity (or similarity) with SEQ ID NO:61, 64, and/or 67 or a portion thereof. In an embodiment, the antibody fragment is represented by an amino acid sequence that comprises or essentially consists of an amino acid sequence having at least 60% sequence identity (or similarity) with SEQ ID NO:61, 64 or 67 or a portion thereof and has a length which is ranged from 76 to 130 amino acids or 80 to 130 or 90 to 120 or 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a sixth embodiment of this third structural feature, the antibody fragment of the invention is represented by an amino acid sequence that comprises or essentially consists of an amino acid sequence having at least 60% sequence identity (or similarity) with SEQ ID NO: 62, 65, 68, 63, 66 and/or 69 or a portion thereof. In an embodiment, the antibody fragment is represented by an amino acid sequence that comprises or essentially consists of an amino acid sequence having at least 60% sequence identity (or similarity) with any of SEQ ID NO: 62, 65, 68, 63, 66 and/or 69 or a portion thereof and has a length which is ranged from 12 to 130 amino acids or 20 to 120 or 30 to 110 or 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72. 73. 74. 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a seventh embodiment of this third structural feature, the antibody fragment of the invention is represented by an amino acid sequence that comprises or essentially consists of an amino acid sequence having at least 60% sequence identity (or similarity) with SEQ ID NO: 24, 32 and/or 40 or a portion thereof. In an embodiment, the antibody fragment is represented by an amino acid sequence that comprises or essentially consists of an amino acid sequence having at least 60% sequence identity (or similarity) with SEQ ID NO:24, 32 and/or 40 or a portion thereof and has a length which is ranged from 110 to 130 amino acids or 100 to 130 or 110 to 120 or 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a eighth embodiment of this third structural feature, the antibody fragment, preferably a VHH or a functional fragment thereof is represented by an amino acid sequence that comprises any of SEQ ID NO:61, 62, 63, 64, 65, 66, 67, 68 and/or 69. In an embodiment, it has a length which is ranged from 12 to 130 amino acids or 20 to 121 or 30 to 121amino acids. More preferably it is represented by an amino acid sequence that comprises SEQ ID NO:24, 32 and/or 40. Even more preferably, it has a length of 118, 119, 120, 121, 122, 123, 124, 125, 126, 127, 128, 129 or 130 amino acids.

### Fourth Structural Feature

The antibody fragment of the invention that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), may also alternatively or in combination be further defined by a fourth structural feature.

In a fourth structural feature, the antibody fragment of the invention, preferably the VHH’s as disclosed herein is represented by an amino acid sequence that comprises at least one combination of CDR sequences chosen from the group comprising:
a CDR1 region having SEQ ID NO: 25, a CDR2 region having has SEQ ID NO: 26, and a CDR3 region having SEQ ID NO: 27, and/or
a CDR1 region having SEQ ID NO: 33, a CDR2 region having has SEQ ID NO: 34, and a CDR3 region having SEQ ID NO: 35, and/or
a CDR1 region having SEQ ID NO: 41, a CDR2 region having has SEQ ID NO: 42, and a CDR3 region having SEQ ID NO: 43 (see table 4).

Thus, in particular embodiments, the present invention provides heavy chain variable domains comprising the heavy chain antibodies with the (general) structure or which is derived therefrom: FR1 - CDR1 - FR2 - CDR2 - FR3 - CDR3 - FR4 in which FR1 to FR4 refer to framework regions 1 to 4, respectively, and in which CDR1 to CDR3 refer to the complementarity determining regions 1 to 3, respectively, and are as further defined herein. SEQ ID NO’s: 24, 32 and 40 (see table 4) give the amino acid sequences of heavy chain variable domains that have been raised against human FOLR1.

TABLE 4

CDR and FR of A3, A4 and A5 as well as other specific fragments thereof (CDR and FR were identified using the IMGT nomenclature as defined later on herein in the general part of the description dedicated to the general definition of the invention)

| | Amino acid sequence |
|---|---|
| A3 (SEQ ID NO:24) | DVQLVESGGGLVQAGGSLRLSCGVSGNMFSMNLMG WYRQPPGKERDWVAVITKDGSTNYADSVKGRFTISRD NAKNTVSLQMNSLKPEDTAVYYCNVQDGSGWIRAWG WDWGQGTQVTVSS |
| CDR of A3 | |
| CDR1 (SEQ ID NO: 25) | GNMFSMNL |
| CDR2 (SEQ ID NO: 26) | ITKDGST |
| CDR3 (SEQ ID NO: 27) | NVQDGSGWIRAWGWD |
| FR1 (SEQ ID NO: 28) | DVQLVESGGGLVQAGGSLRLSCGVS |
| FR2 (SEQ ID NO: 29) | MGWYRQPPGKERDWVAV |
| FR3 (SEQ ID NO: 30) | NYADSVKGRFTISRDNAKNTVSLQMNSLKPEDTAVYYC |
| FR4 (SEQ ID NO: 31) | WGQGTQVTVSS |
| A4 (SEQ ID NO:32) | DVQLVESGGGLARAGGSLRLSCAASGRAFSSTYAMG WFRQAPGKEREFVADINWSEGSPMYIDSVKDRFTISR DNARNSLYLQMNSLKSEDTAVYYCAARRFGQGYEYW GQGTQVTVSS |
| CDR1 (SEQ ID NO: 33) | GRAFSSTYA |
| CDR2 (SEQ ID NO: 34) | INWSEGSP |
| CDR3 (SEQ ID NO: 35) | AARRFGQGYEY |
| FR1 (SEQ ID NO: 36) | DVQLVESGGGLARAGGSLRLSCAAS |
| FR2 (SEQ ID NO: 37) | MGWFRQAPGKEREFVAD |
| FR3 (SEQ ID NO: 38) | MYIDSVKDRFTISRDNARNSLYLQMNSLKSEDTAVYYC |
| FR4 (SEQ ID NO: 39) | WGQGTQVTVSS |
| A5 (SEQ ID NO:40) | DVQLVESGGGLVQAGGSLRLSCGVSGNIFSMNLMGW YRQPPGKEREWVALITKDGSTNYEDSVKGRFTISRDNA KNTVSLQMNSLKPEDTAVYYCNAQDGSGWIRAWGWD WGQGTQVTVSS |
| CDR1 (SEQ ID NO: 41) | GNIFSMNL |
| CDR2 (SEQ ID NO: 42) | ITKDGST |
| CDR3 (SEQ ID NO: 43) | NAQDGSGWIRAWGWD |
| FR1 (SEQ ID NO: 44) | DVQLVESGGGLVQAGGSLRLSCGVS |
| FR2 (SEQ ID NO: 45) | MGWYRQPPGKEREWVAL |
| FR3 (SEQ ID NO: 46) | NYEDSVKGRFTISRDNAKNTVSLQMNSLKPEDTAVYYC |
| FR4 (SEQ ID NO: 47) | WGQGTQVTVSS |
| A3 (SEQ ID NO: 24) | |
| (SEQ ID NO: 61) | NLMGWYRQPPGKERDWVAVITKDGSTNYADSVKGRF TISRDNAKNTVSLQMNSLKPEDTAVYYCNVQDGSGWI RAWGWDW |
| (SEQ ID NO: 62) | NLMGWYRQPPGKERDWVAVITKDGSTNYAD |
| (SEQ ID NO: 63) | NVQDGSGWIRAWGWDW |
| (SEQ ID NO: 64) | AMGWFRQAPGKEREFVADINWSEGSPMYIDSVKDRFT ISRDNARNSLYLQMNSLKSEDTAVYYCAARRFGQGYE YW |
| (SEQ ID NO: 65) | AMGWFRQAPGKEREFVADINWSEGSPMYID |
| (SEQ ID NO: 66) | AARRFGQGYEYW |
| (SEQ ID NO: 67) | NLMGWYRQPPGKEREWV ALlTKDGSTNYEDSVKGRF TISRDNAKNTVSLQMNSLKPEDTAVYYCNAQDGSGWI RAWGWDW |
| (SEQ ID NO: 68) | NLMGWYRQPPGKEREWVALITKDGSTNYED |
| (SEQ ID NO: 69) | NAQDGSGWIRAWGWDW |

### Compound Comprising the Antibody Fragment

In a further aspect, there is provided an antibody fragment, preferably a VHH or a fragment thereof as defined in the previous aspect, wherein said antibody fragment, preferably said VHH or fragment thereof is linked or coupled to an entity such as a moiety. Within the context of the invention, an antibody fragment, preferably a VHH or a fragment thereof which is linked to an entity such as a moiety may be called a compound. Within the context of the application, a compound therefore comprises, essentially consists of or consists of an antibody fragment of the invention and an entity. As earlier disclosed herein, the antibody fragment, preferably a VHH of the invention (or a fragment thereof) is represented by a first, second, third and/or fourth structural feature as identified herein. Alternatively or in combination with said structural feature, said antibody fragment, preferably a VHH of the invention (or a fragment thereof) is characterized by a functional feature which is to specifically bind human folate receptor alpha (FOLR1), but not murine FOLR1, not human folate receptor beta (FOLR2) and not human folate receptor gamma (FOLR3) (or by an antibody fragment specifically binding human FOLR1, but neither binding human FOLR2 nor human FOLR3) . In an embodiment, the antibody fragment, preferably a VHH of the invention (or a fragment thereof) is characterized by a functional feature which is to specifically bind human folate receptor alpha (FOLR1), but not murine FOLR1, not human folate receptor beta (FOLR2) and not human folate receptor gamma (FOLR3) (or by an antibody fragment specifically binding human FOLR1, but neither binding human FOLR2 nor human FOLR3). An antibody fragment, VHH or fragment of a VHH of the invention should therefore fulfil at least one of the structural features and/or functional features.

The entity and the antibody fragment may be linked or coupled to each other. An entity may be a cell as explained later herein. When the entity is a cell, the expression “antibody fragment linked or coupled to an entity” means that the antibody fragment is expressed in said cell. Nucleic acid molecules encoding the antibody fragment of the invention are disclosed later herein.

The identity of the moiety and/or the type of link may vary depending on the type of applications envisaged for the antibody fragment or for the moiety or for the compound. A moiety may be a molecule or a label as defined herein.

### Compound for Brain Targeting Applications

In an embodiment, the moiety linked to the antibody fragment (preferably a VHH or a fragment thereof) is a molecule to be delivered to the central nervous system (CNS). In the context of the invention, a “compound” is or comprises or essentially consists of or consists of an antibody fragment, preferably a VHH or a fragment thereof (all as defined herein), wherein said antibody fragment is linked to a moiety, preferably a molecule to be delivered to the CNS. More preferably, the molecule is a medicament acting in the CNS, preferably acting in the brain. Even more preferably, the molecule crosses the brain blood barrier (BBB) and/or the blood-cerebrospinal fluid barrier (BCSFB) via transport via the human FOLR1; a mechanism called receptor mediated transcytosis (RMT). Any moiety, molecule or medicament known to act in the CNS or in the brain is potentially encompassed by the present invention to be linked to the antibody fragment of the invention. Such a moiety, molecule or medicament that may be linked to an antibody fragment of the invention may be any molecule or medicament, which does not cross the brain blood barrier (BBB) on its own. The molecule may be a peptide, a small molecule or a nucleic acid. A peptide may be a cytokine. A small molecule may be a chemotherapeutic. An entity may be a cell such as a CAR-T cell, a CAR-NK cell, a BITE or a LITE.

In an embodiment, a medicament acting in the brain is a medicament for preventing and/or treating choroid plexus papilloma and/or hydrocephalus. Folate receptor alpha (FOLR1) is overexpressed in some human cancers, inclusing choroid plexus papilloma or tumor. An increased expression of folate receptor alpha (FOLR1) in the brain has been associated with hydrocephalus.

In an embodiment, the moiety linked to the antibody fragment is an aziridinyl-epothilone such as described in WO2007140297A2, preferably the moiety linked to the antibody fragment is 7,11-dihydroxy-17-[2- hydroxyethyl]-8,8,10,12-tetramethyl-3-[1-methyl-2-(2-methyl- 4- thiazolyl)ethenyl]-4-oxa-17-azabicyclo[14.1.0]heptadecane-5,9-dione, or a derivative thereof, or a pharmaceutically acceptable salt thereof, more preferably said moiety linked to the antibody fragment is a compound for the treatment and/or prevention of chorois plexus papilloma.

In an embodiment, the moiety linked to the antibody fragment is a 1H-pyrrolo[3,2-b]pyridine derivative such as described in WO2014145051A1, preferably the moiety linked to the antibody fragment is 4-(6-(3,5-dimethylisoxazol-4-yl)-1-(1-(pyridin-2-yl)ethyl)-1H-pyrrolo[3,2-b]pyridin-3-yl)benzoic acid, or 4-(6-(3,5-dimethylisoxazol-4-yl)-1-(phenyl(pyridin-2-yl)methyl)-1H-pyrrolo[3,2-b]pyridin-3-yl)benzoic acid, or methyl 2-(4-(6-(3,5-dimethylisoxazol-4-yl)-1-(1-(pyridin-2-yl)ethyl)-1H-pyrrolo[3,2-b]pyridin-3-yl)phenyl)acetate, or methyl 4-(6-(3,5-dimethylisoxazol-4-yl)-1-(pyridazin-3-ylmethyl)-1H-pyrrolo[3,2-b]pyridin-3-yl)benzoate, or N-cyclopropyl-4-(6-(3,5-dimethylisoxazol-4-yl)-1-(1-(pyridin-2-yl)ethyl)-1H-pyrrolo[3,2-b]pyridin-3-yl)benzenesulfonamide, or (4-(6-(3,5-dimethylisoxazol-4-yl)-1-(phenyl(pyridin-2-yl)methyl)-1H-pyrrolo[3,2-b]pyridin-3-yl)phenyl)methanol, or 3,5-dimethyl-4-(1-(pyridazin-3-ylmethyl)-3-(1-(2,2,2-trifluoroethyl)-1H-pyrazol-4-yl)-1H-pyrrolo[3,2-b]pyridin-6-yl)isoxazole, or 3,5-dimethyl-4-(3-(1-methyl-1H-pyrazol-4-yl)-1-(1-(pyridin-2-yl)ethyl)-1H-pyrrolo[3,2-b]pyridin-6-yl)isoxazole, or a derivative thereof, or a pharmaceutically acceptable salt thereof, more preferably said moiety linked to the antibody fragment is a compound for the treatment and/or prevention of chorois plexus papilloma. In an embodiment, the moiety linked to the antibody fragment is a 1H-pyrrolo[3,2-b]pyridine derivative such as described in WO2017053243A1, preferably the moiety linked to the antibody fragment is 4-(1-(1,1-di(pyridin-2-yl)ethyl)-6-(3,5-dimethylisoxazol-4-yl)-1H-pyrrolo[3,2-b]pyridin-3-yl)benzoic acid, or 4-(1-(cyanodipyridin-2-ylmethyl)-6-(3,5-dimethylisoxazol-4-yl)-1H-pyrrolo[3,2-b]pyridin-3-yl)benzoic acid, or 4-(6-(3,5-dimethylisoxazol-4-yl)-1-(fluorodipyridin-2-ylmethyl)-1H-pyrrolo[3,2-b]pyridin-3-yl)benzoic acid, or a derivative thereof, or a pharmaceutically acceptable salt thereof, more preferably said moiety linked to the antibody fragment is a compound for the treatment and/or prevention of chorois plexus papilloma.

In an embodiment, the moiety linked to the antibody fragment is an pyrrolidine sulfonamide TRPC4 antagonists such as described in WO2018055524A1, preferably the moiety linked to the antibody fragment is 2-(((3R,4S)-4-(4-chlorophenoxy)-3-hydroxy-3-(hydroxymethyl)pyrrolidin-1-yl)sulfonyl)-5-(trifluoromethyl)benzonitrile, or 4-(((3S,4S)-1-((2-cyano-4-(trifluoromethyl)phenyl)sulfonyl)-4-hydroxy-4-((S)-1- hydroxyethyl)pyrrolidin-3-yl)oxy)-2-fluorobenzonitrile, or a derivative thereof, or a pharmaceutically acceptable salt thereof, more preferably said moiety linked to the antibody fragment is a compound for the treatment and/or prevention of hydrocephalus.

In an embodiment, the moiety linked to the antibody fragment is a sulfonpyrazole or sulfonylpyrazoline carboxamidine 5-HT6 antagonist such as described in WO2008034863A2, preferably the moiety linked to the antibody fragment is N′-(1-acetylindolin-5-ylsulfonyl)-N,4-diethyl-4,5-dihydro-1H-pyrazole-1-carboximidamide, or N′-(4-(1H-pyrazol-1-yl)phenylsulfonyl)-N,4-diethyl-4,5-dihydro-1H-pyrazole-1-carboximidamide, or N-((5-(N-((4-ethyl-4,5-dihydro-1H-pyrazol-1-yl) (ethylamino)methylene)sulfamoyl)thiophen-2-I)methyl)benzamide, or N′-(5-(5-(chloromethyl)-1,2,4-oxadiazol-3-yl)thiophen-2-ylsulfonyl)-N,4-diethyl-4,5-dihydro-1H-pyrazole-1-carboximidamide, or a derivative thereof, or a pharmaceutically acceptable salt thereof, more preferably said moiety linked to the antibody fragment is a compound for the treatment and/or prevention of hydrocephalus.

In an embodiment, the moiety linked to the antibody fragment is an arylsulfonyl pyrazoline carboxamidine 5-HT6 antagonist such as described in WO2009115515A1, preferably the moiety linked to the antibody fragment is N′-(4-amino-3-chlorophenylsulfonyl)-N-ethyl-2,3,8-triazaspiro[4.5]dec-3-ene-2-carboximidamide, or N′-(4-amino-3-chlorophenylsulfonyl)-N-ethyl-2,3-diazaspiro[4.4]non-3-ene-2-carboximidamide, or N′-(4-amino-3-chlorophenylsulfonyl)-N-ethyl-4,4-dimethyl-4,5-dihydro-1H-pyrazole-1-carboximidamide, or N′-(4-aminophenylsulfonyl)-2,3-diazaspiro[4.4]non-3-ene-2-carboximidamide, or N′-(4-aminophenylsulfonyl)-8-oxa-2,3-diazaspiro[4.5]dec-3-ene-2-carboximidamide, or N′-(4-aminophenylsulfonyl)-N,4-diethyl-4,5-dihydro-1H-pyrazole-1-carboximidamide, or N′-(4-aminophenylsulfonyl)-N-ethyl-5-phenyl-4,5-dihydro-1H-pyrazole-1-carboximidamide, or N′-(4-aminophenylsulfonyl)-N-methyl-8-oxa-2,3-diazaspiro[4.5]dec-3-ene-2-carboximidamide, or a derivative thereof, or a pharmaceutically acceptable salt thereof.

In an embodiment, the moiety linked to the antibody fragment is a cyclodextrin-API conjugate such as described in WO2013116200A1, more preferably said moiety linked to the antibody fragment is a compound for the treatment and/or prevention of hydrocephalus.

In an embodiment, the moiety linked to the antibody fragment is pyrrolidine sulfonamide TRPV4 antagonists such as described in WO2018055527A1, preferably the moiety linked to the antibody fragment is 4-(((3S,4R)-1-((2,4-dichlorophenyl)sulfonyl)-4-hydroxy-4-(hydroxymethyl)pyrrolidin-3-yl)methyl)-2-fluorobenzonitrile, or 4-(((3S,4R)-1-((2-chloro-4-(trifluoromethyl)phenyl)sulfonyl)-4-hydroxy-4- (hydroxymethyl)pyrrolidin-3-yl)methyl)benzonitrile, or 4-(((3S,4R)-1-((5-chloropyridin-2-yl)sulfonyl)-4-hydroxy-4-(hydroxymethyl)pyrrolidin-3- yl)methyl)-3-(2,2,2-trifluoroethoxy)benzonitrile, or 4-(((3S,4S)-1-((2,4-dichlorophenyl)sulfonyl)-4-hydroxy-4-(hydroxymethyl)pyrrolidin-3- yl)methyl)-2-fluorobenzonitrile, or 4-(((3S,4R)-1-((2-cyano-4-(trifluoromethyl)phenyl)sulfonyl)-4-hydroxy-4-(hydroxymethyl)pyrrolidin-3-yl)methyl)-2-fluorobenzonitrile, or 4-(((3S,4R)-1-((2-chloro-4-cyanophenyl)sulfonyl)-4-hydroxy-4-(hydroxymethyl)pyrrolidin-3- yl)methyl)-2-fluorobenzonitrile, or 4-(((3S,4S)-4-(aminomethyl)-1-((5-chloropyridin-2-yl)sulfonyl)-4-hydroxypyrrolidin-3- yl)methyl)-2-fluorobenzonitrile, or a derivative thereof, or a pharmaceutically acceptable salt thereof, more preferably said moiety linked to the antibody fragment is a compound for the treatment and/or prevention of hydrocephalus.

In an embodiment, the moiety linked to the antibody fragment is [5-(5-chloro-1H-pyrrolo[2,3-b]pyridin-3-ylmethyl)-pyridin-2-yl]- (6-trifluoromethyl-pyridin-3-ylmethyl)-amine such as described in WO2016179415A1, or a derivative thereof, or a pharmaceutically acceptable salt thereof, preferably said moiety linked to the antibody fragment is a compound for the treatment and/or prevention of hydrocephalus.

In an embodiment, the moiety linked to the antibody fragment is a N-(pyridin-3-ylmethyl) substituted 1H-pyrrolo[2,3-b]pyridine derivative such as described in WO2013142427A1 or a 1H-pyrrolo[2,3-b]pyridine derivatives such as described in WO2017100201A1, or a derivative thereof, or a pharmaceutically acceptable salt thereof, preferably said moiety linked to the antibody fragment is a compound for the treatment and/or prevention of hydrocephalus.

WO2007140297A2, WO2014145051A1, WO2017053243A1, WO2018055524A1, WO2008034863A2, WO2009115515A1, WO2013116200A1, WO2018055527A1, WO2016179415A1, WO2013142427A1, and WO2017100201A1 are incorporated in their entirety, and all compounds disclosed therein may be a moiety linked to the antibody fragment in the context of the current application.

### Labelled Compound

In a further aspect, there is provided a labelled compound that comprises or consists of or essentially consists of an antibody fragment, preferably a heavy chain antibody (VHH) or a fragment thereof, which specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein said antibody fragment is linked to a moiety which is a label.

In an embodiment, the label is a radionuclide (i.e., a radioactive label). Processes for labelling the antibody fragment to a radionuclide are disclosed in detail in the definition part at the end of the description. In a preferred embodiment, an antibody fragment, preferably a heavy chain antibody (VHH) or a fragment thereof, which specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3) is linked to a moiety and the moiety is a radionuclide.

In this aspect, the antibody fragment, preferably a heavy chain antibody (VHH) or a fragment thereof, which is coupled to a radionuclide may be called a labelled or a radiolabelled compound.

In an embodiment, the antibody fragment in such labelled compound fulfils at least one of the structural features earlier defined herein: first, second, third, and fourth structural features.

Examples of suitable radionuclides which can be linked to the antibody fragment of the invention especially for therapeutic applications, preferably a VHH as disclosed herein can for example without any limitation be chosen from the group consisting of α-emitting radioisotopes and β⁻-emitting radioisotopes, including but not limited to a radioisotope chosen from the group consisting of Actinium-225, Astatine-211, Bismuth-212, Bismuth-213, Caesium-137, Chromium-51, Cobalt-60, Copper-67, Dysprosium-165, Erbium-169, Fermium-255, Gold-198, Holium-166, lodine-125, lodine-131, Iridium-192, Iron-59, Lead-212, Lutetium-177, Molybdenum-99, Palladium-103, Phosphorus-32, Potassium-42, Rhenium-186, Rhenium-188, Samarium-153, Radium-223, Radium-224, Ruthenium-106, Sodium-24, Strontium-89, Scandium-47, Terbium-149, Terbium-161, Terbium-149, Thorium-227, Xenon-133, Ytterbium-169, Ytterbium-177 and Yttrium-90.

In still further particular embodiments, the radionuclide present in the labelled compound as disclosed herein is lodine-131.

In still further particular embodiments, the radionuclide present in the labelled compound as disclosed herein is Actinium-225.

In still further particular embodiments, the radionuclide present in the labelled compound as disclosed herein is Lutetium-177.

Examples of suitable radionuclides which can be linked to the antibody fragment of the invention especially for diagnostic applications, preferably a VHH as disclosed herein can for example without any limitation be chosen from the group consisting of positron-emitting radioisotopes (PET) or γ-emitting radioisotopes (SPECT), including chosen from the group consisting of: lodine-131, Yttrium-90, lodine-125, Lutetium-177, Rhenium-186, Rhenium-188, Terbium-161, Technetium-99m, Indium-111, Xenon-133, Thallium-201, Fluorine-18, Scandium-43, Scandium-44, Gallium-68, Gallium-67, Copper-67, lodine-123, lodine-124, Zirconium-89 and Copper-64.

In still further particular embodiments, the radionuclide present in the labelled compound as disclosed herein is Technetium-99m.

In still further particular embodiments, the radionuclide present in the labelled compound as disclosed herein is lodine-131.

In still further particular embodiments, the radionuclide present in the labelled compound as disclosed herein is Actinium-225.

In still further particular embodiments, the radionuclide present in the labelled compound as disclosed herein is Lutetium-177.

Examples of suitable radionuclides which can be linked to the antibody fragment of the invention especially for theranostic (i.e., diagnostic and therapeutic) applications, preferably a VHH as disclosed herein can for example without any limitation be chosen from the group consisting of: Actinium-225, Bismuth-213, lodine-131, lodine-125, Lutetium-177, Yttrium-90, Copper-67, Terbium-161, Rhenium-186 and Rhenium-188.

In an embodiment, the linker separating the antibody fragment, preferably a heavy chain antibody (VHH) or a fragment thereof, is a benzoate linker. Preferably, this benzoate linker comprises N-succinimidyl-4-guanidinomethyl-3-[l-131]iodobenzoate (SGMIB) or a suitable derivative thereof. Alternatively, 2-[Bis[2-[bis(carboxymethyl)amino]ethyl]amino]acetic acid (DTPA), 1,4,7,10-Tetraazacyclododecane-1,4,7,10-tetraacetic acid (DOTA) or N,N′-bis[(6-carboxy-2-pyridil)methyl]-4,13-diaza-18-crown-6 (MACROPA) or a derivative thereof may be used.

In an embodiment, the labelled compound is:
the antibody fragment as defined earlier herein which is linked via SGMIB to lodine-131,
the antibody fragment as defined earlier herein which is linked via DOTA to Lutetium-177,
the antibody fragment as defined earlier herein which is linked via DOTA to Actinium-225 or
the antibody fragment as defined earlier herein which is linked to Technetium-99m.

Each of these labelled compounds had been synthesized and tested in the experimenal part.

In a preferred embodiment, the labelled compound is:
an antibody fragment that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein the epitope comprises the linear amino acid stretch or region 89-94, 140-150 and/or 176-184 of SEQ ID NO:1, said antibody fragment being linked via SGMIB to Iodine-131,
an antibody fragment that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein the epitope comprises the linear amino acid stretch or region 89-94, 140-150 and/or 176-184 of SEQ ID NO:1, said antibody fragment being linked via DOTA to Lutetium-177,
an antibody fragment that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein the epitope comprises the linear amino acid stretch or region 89-94, 140-150 and/or 176-184 of SEQ ID NO:1, said antibody fragment being linked via DOTA to Actinium-225 or
an antibody fragment that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein the epitope comprises the linear amino acid stretch or region 89-94, 140-150 and/or 176-184 of SEQ ID NO:1, said antibody fragment being linked to Technetium-99m.

In a preferred embodiment, the labelled compound is:
an antibody fragment, that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein the epitope comprises the combination of linear amino acid stretches or regions 89-94, 140-150 and 176-184 of SEQ ID NO:1, said antibody fragment being linked via SGMIB to lodine-131,
an antibody fragment, that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein the epitope comprises the combination of linear amino acid stretches or regions 89-94, 140-150 and 176-184 of SEQ ID NO:1, said antibody fragment being linked via DOTA to Lutetium-177,
an antibody fragment, that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein the epitope comprises the combination of linear amino acid stretches or regions 89-94, 140-150 and 176-184 of SEQ ID NO:1, said antibody fragment being linked via DOTA to Actinium-225 or
an antibody fragment, that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein the epitope comprises the combination of linear amino acid stretches or regions 89-94, 140-150 and 176-184 of SEQ ID NO:1, said antibody fragment being linked to Technetium-99m.

In a preferred embodiment, the labelled compound is:
an antibody fragment that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence, said antibody fragment being linked via SGMIB to lodine-131,
an antibody fragment that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence, said antibody fragment being linked via DOTA to Lutetium-177,
an antibody fragment that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence, said antibody fragment being linked via DOTA to Actinium-225 or,
an antibody fragment that specifically binds an epitope of human folate receptor alpha (FOLR1), but does neither bind murine FOLR1 nor human folate receptor beta (FOLR2) nor human folate receptor gamma (FOLR3) (or an antibody fragment specifically binding human FOLR1, but neither binding human FOLR2 nor human FOLR3), wherein said antibody fragment is represented by an amino acid sequence that comprises an amino acid sequence having at least 63% sequence identity with at least one of SEQ ID NO:8, 9, 2, 3, 4, 5, 6, or 7 over the full length of said sequence or over at least 50% of the length of said sequence, said antibody fragment being linked to Technetium-99m.

The labelled compound is specifically directed against human FOLR1 which is considered as a cancer antigen. The labelled compound may be used as a diagnostic molecule and/or as a therapeutic molecule. In an embodiment, the disease diagnosed or treated is cancer.

As used herein, human FOLR1 is considered a ‘cancer cell-specific antigen’, ‘cancer-specific antigen’, ‘cancer antigen’, ‘target protein present on, “target protein expressed in” and/or “specific for a cancer cell’, ‘cancer cell-specific target (protein)”, “cancer (cell)-associated antigen” are used interchangeably herein and refers to the fact that human FOLR1 is mainly present on (or mainly expressed on) cancer cells and not on any other cell. For example, human FOLR1 is expressed in ovarian, endometrial, brain, lung, adrenal carcinoma, head and neck, breast, stomach, colon-rectum cancer cells.

As used herein, the term “FOLR1 positive” or “expressing FOLR1” or “overexpressing FOLR1” may refer to cancerous or malignant human cells or tissue characterized by FOLR1 protein overexpression and thus have abnormally high levels of the FOLR1 gene and/or the FOLR1 protein compared to normal healthy cells. In this context, “overexpressing” may mean that the expression is at least 10%, 20%, 30%, 40%, 50%, 60%, 70%, 80%, 90%, 100% or more than the expression in a control cell line. A control cell line may be a healthy or non-diseased cell.

In another embodiment, the label present in the labelled compound is a non-radioactive label. In an embodiment, such non-radioactive label is a fluorescent label. This non-radioactive labelled compound may be used for diagnostic applications as defined herein. Examples of suitable fluorescent labels include Alexafluor variants, Cy3, Cy5, FITC (fluorescein), Coumarin, Texas red, Oregon Green, Pacific Blue, Pacific Green, Pacific Orange, PE-Cyanine7, PerCP-Cyanine5.5, TRITC (tatramethylrhodamine). All may be obtained from ThermoFisher.

### Composition

In a further aspect there is provided a composition comprising or consisting essentially of an antibody fragment, such as a VHH or a fragment thereof. In a further aspect, there is also provided a composition comprising or consisting essentially of a compound, when said antibody fragment is linked to an entity such as a moiety. This moiety can be either a molecule active in the CNS (preferably in the brain) or a label (i.e., radionuclide or non-radioactive). In an embodiment, the composition comprises or consists essentially of a labelled compound as earlier defined herein. The composition comprises an excipient. The excipient should be acceptable for diagnostic and/or therapeutic purpose. In an embodiment the composition is a pharmaceutical composition. In another embodiment, the composition is a diagnostic composition. It is also encompassed by the invention that the composition is a pharmaceutical and diagnostic composition. Suitable formulations of the invention are disclosed in the definition part at the end of the description.

A diagnostic composition as defined herein may comprise a screening dose or a stratification dose and may therefore be called a screening composition or a stratification composition.

The pharmaceutical compositions as envisaged herein can be used in the prevention and/or treatment of diseases and disorders associated with human FOLR1. In an embodiment, the disease is a cancer associated with expression or overexpression of human FOLR1. In particular, the application provides pharmaceutical compositions that are suitable for prophylactic and/or therapeutic use in a warm-blooded animal, and in particular in a mammal, and more in particular in a human being.

### Kit

In a further aspect there is also provided a kit. Such as a kit is suitable for diagnostic and therapeutic applications as described herein. Such applications include the use of the antibody fragment of the invention, of the compound of the invention comprising the antibody fragment linked to an entity such as a moiety, said moiety being a radioactive or a non-radioactive label. A more detailed definition of the kit is provided in the part dedicated to the definition at the end of the description.

### Diagnostic Use of the Antibody Fragment and of the Labelled Compound

In a further aspect, there is provided a method wherein the antibody fragment or the labelled compound or a composition comprising it is used to assess expression of human FOLR1 in a subject or in an isolated sample of said subject. This method may comprise the following steps:
a) providing an antibody fragment or a labelled compound as identified herein,
b) administering it to a subject or contacting it with an isolated sample of a subject,
c) assessing the expression of human FOLR1 in said subject or in said isolated sample of said subject.

This method may be called a diagnostic method. This method may be an in vitro or an in vivo method. This method may allow the localization of the expression of human FOLR1 in a subject or in an isolated sample of said subject and may allow the prediction and/or prognosis of a certain disease and/or disorder and/or condition in said subject. In an embodiment, this method may be a stratification method to identify patients that are likely to respond to a particular (cancer) treatment. Therefore, in a further aspect, there is provided a method wherein the antibody fragment or the labeled compound or a composition comprising it is used to stratify the subject and assess whether he will be likely to respond to a particular (cancer) treatment. This method may comprise the following steps:
a) providing an antibody fragment or a labelled compound as identified herein,
b) administering it to a subject or to an isolated sample of a subject,
c) assessing the expression of human FOLR1 in said subject or in said isolate sample of said subject and
d) deciding whether the subject is likely to be responsive to a medical treatment such as the one comprising the labelled compound of the invention.

The antibody fragment of the invention may be used in such a diagnostic method. The antibody fragment of the invention does not per se need to be coupled to a label in order to be used in such a method. Such a method may be an ELISA.

Optionally if the method defined above is carried out using a radioactive labelled compound, the radioactive labelled compound or a version thereof suited for therapy is administrated to the subject as a treatment. The subject is preferably a human being. Each and every radioactive labelled compound as defined earlier herein is suitable in this method. Detailed information is disclosed in the definition part at the end of the description in order to produce/provide and in order to administer a labelled compound as identified herein. The administration of a labelled compound for diagnostic purpose and for therapeutic purpose is similar. A method according to this aspect may be an in vitro, ex vivo method.

In an embodiment, a screening dose or a biomarker dose is administrated to a subject or to an isolated sample of said subject. Detailed definitions are provided later on especially by comparison to the definition of a therapeutic dose.

In an embodiment of the diagnostic method, the labelled compound is:
the antibody fragment as defined earlier herein linked via SGMIB to lodine-131,
the antibody fragment as defined earlier herein linked via DOTA to Lutetium-177,
the antibody fragment as defined earlier herein linked to Technetium-99m.

Each of these labelled compounds had been synthesized and tested in the experimenal part.

The assessment of the expression of human FOLR1 in the subject is preferably carried out using imaging as disclosed in the part dedicated to definition at the end of the description. Alternatively, the assessment of the expression of human FOLR1 in the subject is preferably carried out using an isolated sample of the subject. Within the context of the invention, an isolated sample of a subject may be a tissue or a liquid sample from said subject. A liquid may be serum. An isolated sample from a patient may be called a biopsy or a tumor biopsy.

### Therapeutic Use of the (Labelled) Compound

In a further aspect there is provided an antibody fragment, preferably a VHH or a fragment thereof or a compound or a labelled compound or a composition (all as defined herein) for use as a medicament.

In an embodiment, the compound comprises an entity such as a moiety linked to the antibody fragment (preferably a VHH or a fragment thereof) and said moiety is a molecule to be delivered to the central nervous system (CNS). The molecule preferably does not cross the brain blood barrier (BBB) on its own. The molecule may be a peptide or a small molecule, a nucleic acid. A peptide may be a cytokine. A small molecule may be a chemotherapeutic. An entity may be a cell such as a CAR-T cell, a CAR-NK cell, a BITE or a LITE. This compound or a composition comprising it may be a medicament for treating a disease or condition associated with the brain or wherein the medicament is for treating a disease or condition wherein an alteration of a brain activity will impact another organ of the subject.

More preferably, the molecule is a medicament acting in the CNS, preferably acting in the brain. Even more preferably, the molecule crosses the brain blood barrier (BBB) and/or the blood-cerebrospinal fluid barrier (BCSFB) via transport via the human FOLR1; a mechanism called receptor mediated transcytosis (RMT). Any medicament known to act in the CNS or in the brain is potentially encompassed by the present invention.

A disease or a condition or a disorder associated with the CNS or the brain may be any disease, condition or disorder known to the skilled person as being associated with the CNS or the brain. Such disease, condition or disorder may reflect an altered CNS activity or an altered brain activity. Examples of such disease, condition or disorder include: epilepsy, autism spectrum disorders, autism, altered food intake, altered heat regulation, altered pain sensation, chronic pain, depression, migraines, hearing loss, bipolar disorders, alzheimer’s disease, schizophrenia, brain injury, blindness, stroke, parkinson’s disease, multiple sclerosis, spinal cord injury, amyotrophic lateral sclerosis.

In another embodiment, the compound is a labelled compound and said labelled compound or a composition comprising the same is a medicament for treating a cancer. In an embodiment, said cancer is associated with an expression of human FOLR1 on a cancer or a tumour cell or a metastasized lesion. The cancer treated may be metastatic, preferably wherein a metastatic cell is found in the brain. This is an attractive embodiment as the labelled compound is able to cross the BBB.

A cancer associated with expression of FOLR1 may be any of an ovarian, endometrial, brain, lung, adrenal carcinoma, head and neck, breast, stomach, colon-rectum cancer. However, the invention is not limited to these types of cancer. As soon as a subject is suspected to have a cancer cell expressing or overexpressing human FOLR1, the labelled compound or a composition comprising the same may be used.

In an embodiment, the subject has been first diagnosed using a labelled compound of the invention before being treated with the same or with a distinct label compound. The identity of the nuclide may not be the same in diagnostic and therapy applications.

In an embodiment of this therapeutic method or use, the labelled compound is:
the antibody fragment as defined earlier herein linked via SGMIB to lodine-131,
the antibody fragment as defined earlier herein linked via DOTA to Lutetium-177 or
the antibody fragment as defined earlier herein linked via DOTA to Actinium-225.

Each of these labelled compounds had been synthesized and tested in the experimenal part.

Within the context of the invention, a disease or condition or disorder has been prevented or treated when the administration of a compound respectively a labelled compound has been carried out and has resulted:
in the improvement of at least one symptom associated with said disease or condition or disorder and/or
in the improvement of at least one parameter associated with said disease or condition or disorder.

The improvement may be observed at least one day, two days, three days, four days, five days, six days, one week after the compound, respective labelled compound has been administrated. Alternatively, the improvement may be observed at least one month, six months after the administration of the compound, respectively the labelled compound. Envisaged doses and administration modes are further disclosed in the definition part at the end of the description.

A labelled compound or a composition comprising the same exhibits an anti-cancer activity when at least one of the following is fulfilled:
it can kill a tumour cell or cancer cell that expresses human FOLR1,
It can reduce or slow the growth and/or proliferation of such a tumour cell or cancer cell.
It can reduce the size of a primary tumour or of a metastatic lesion,
It can delay the occurrence of metastases and/or of tumour cell migration,
It can delay the increase of a tumour weight or growth, and
It can extend patient survival of at least one month, several months or more (compared to those not treated or treated with a control or compared with the subject at the onset of the treatment).

An anti-cancer activity may have been identified or determined when the number of viable cancer cells, and/or viable tumor cells after the administration of the labelled compound is less than 90%, less than 80%, less than 70%, less than 60%, less than 50%, less than 40%, less than 30%, less than 20%, less than 10% of the number of initial viable cancer cells and/or initial viable tumour cells.

An anti-cancer activity may have been identified or determined when the size of a primary tumour and/or the size of a metastatic lesion after the administration of the labelled compound is less than 90%, less than 80%, less than 70%, less than 60%, less than 50%, less than 40%, less than 30%, less than 20%, less than 10% of the size of said primary tumour and/or of the size of said metastatic lesion.

Tumor cell death may be assessed by measurement of radiolabeled Annexin A5, a molecular imaging agent to measure cell death in vitro, and non-invasively in patients with cancer such as ICH (Schutters K. et al., Apoptosis 2010 and de Saint-Hubert M. et al., Methods 48: 178, 2009). ICH has been defined in the definition part at the end of the description.

Tumor growth may be inhibited at least 5%, 10%, 20%, 30%, 40%, 50%, 55%, 60%, 65%, 70% or 75%, or more. Tumor growth may be assessed using techniques known to the skilled person. Tumor growth may be assessed using MRI (Magnetic Resonance Imaging) or CT (Computer Tomography).

In certain embodiments, tumor weight increase or tumor growth may be inhibited at least 20%, 30%, 40%, 50%, 55%, 60%, 65%, 70% or 75%, or more. Tumor weight or tumor growth may be assessed using techniques known to the skilled person. The detection of tumor growth or the detection of the proliferation of tumor cells may be assessed in vivo by measuring changes in glucose utilization by positron emission tomography with the glucose analogue 2-[18F]-fluor-2-deoxy-D-glucose (FDG-PET) or [18F]-′3-fluoro-′3-deoxy-L-thymidine PET. An ex vivo alternative may be staining of a tumor biopsy with Ki67.

A delay in occurrence of metastases and/or of tumor cell migration may be a delay of at least one week, one month, several months, one year or longer. The presence of metastases may be assessed using MRI, CT or Echography or techniques allowing the detection of circulating tumour cells (CTC). Examples of the latter tests are CellSearch CTC test (Veridex), an EpCam-based magnetic sorting of CTCs from peripheral blood.

In certain embodiments, tumor growth may be delayed or inhibited at least one day, two days, three days, four days, five days, six days or one week, two weeks, three weeks, one month, two months or more. In a certain embodiment, an occurrence of metastases is delayed at least one week, two weeks, three weeks, four weeks, one months, two months, three months, four months, five months, six months or more.

The labelled compound of the invention exerts its anti-cancer activity through the mechanism of radiotoxicity once it is bound to a cancer or tumour cell or metastatic lesion expressing human FOLR1. Tumour cell, cancer cell, lesion, metastatic lesion and dose of the labelled compound have been defined in the section entitled definition.

In a further aspect, there is provided a method for the prevention and/or treatment of a disease and/or disorder and/or condition comprising administering to a subject in need thereof, an antibody fragment, preferably a VHH or a fragment thereof or a compound or a labelled compound or a composition as envisaged herein. All features of this method have been defined earlier herein.

### Non-Human Mammal

In a further aspect, there is provided a non-human animal comprising a nucleic acid construct allowing the expression of human FOLR1. A non-human animal may be a mammal. Preferred mammals include mouse, rat, rabbit. Such animal may be obtained using common knowledge techniques known to the skilled person. In an embodiment, such non-human animal may have been modified to no longer express its endogenous FOLR1. In an embodiment, the endogenous FOLR1 of the non-human animal has been replaced by the human FOLR1 gene. The human FOLR1 coding nucleic acid is represented by SEQ ID NO:58. This gene replacement may be carried out by homologous recombination as known to the skilled person. In an embodiment, the targeting vector used comprises SEQ ID NO:59. In a preferred embodiment, the non-human animal is a mouse and the targeting vector comprising SEQ ID NO:59 has been introduced into it using techniques known to the skilled person. The resulting mouse does no longer express murine FOLR1 and instead thereof expresses human FOLR1. In an embodiment, the mouse has been obtained as described in example 2. In an embodiment, the expression of human FOLR1 is assessed in said non-human animal by the labeled compound of the invention. Alternatively, it can be assessed using other antibodies or antibody fragments known to be specific for human FOLR1. Such commercial anti-human FOLR1 antibody may be IgG1 clone #548908 (R&D systems #MAB5646). Once the expression of human FOLR1 has been validated in this non-human animal, it can be used to assess the functionality of an antibody fragment, a compound or of a labeled compound of the invention. This non-human animal may therefore be used in a method for screening a molecule specifically binding human FOLR1, preferably a new antibody fragment or a compound of the invention. This non-human animal is a quite interesting model and surprisingly it is viable: although the mouse deficient for the murine FOLR1 has a lethal phenotype at the embryonic stage, this human FOLR1 knock-in mouse is viable. It means that although murine and human FOLR1 are not both recognized by the antibody fragment of the invention, human FOLR1 is able to at least partially compensate for the loss of expression of its murine counterpart.

### DEFINITIONS

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise.

The terms ‘comprising’, ‘comprises’ and ‘comprised of as used herein are synonymous with ‘including’, ‘includes’ or ‘containing’, ‘contains’, and are inclusive or open-ended and do not exclude additional, non-recited members, elements or method steps. The expression “essentially consists of” used in the context of a product or a composition (“a product essentially consisting of” or “a composition essentially consisting of”) means that additional molecule may be present but that such molecule does not change/alter the characteristic/activity/functionality of said product or composition. For example, a composition may essentially consist of an antibody fragment if the composition as such would exhibit similar characteristic/activity/functionality as the one of the antibody fragment.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/-10% or less, preferably +/-5% or less, more preferably +/-1% or less, and still more preferably +/-0.1% or less of and from the specified value, insofar such variations are appropriate to perform in the disclosed invention. It is to be understood that the value to which the modifier ‘about’ refers is itself also specifically, and preferably, disclosed.

### Polypeptide/Nucleic Acid Molecule and Identity/Similarity

As used herein, amino acid residues will be indicated either by their full name or according to the standard three-letter or one-letter amino acid code.

As used herein, the terms ‘polypeptide’ or ‘protein’ are used interchangeably, and refer to a polymeric form of amino acids of any length, which can include coded and non-coded amino acids, chemically or biochemically modified or derivatized amino acids, and polypeptides having modified peptide backbones. A “peptide” is also a polymer of amino acids with a length which is usually of up to 50 amino acids. A polypeptide or peptide is represented by an amino acid sequence.

As used herein, the terms ‘nucleic acid molecule’, ‘polynucleotide’, ‘polynucleic acid’, ‘nucleic acid’ are used interchangeably and refer to a polymeric form of nucleotides of any length, either deoxyribonucleotides or ribonucleotides, or analogs thereof. A nucleic acid molecule is represented by a nucleic acid sequence, which is primarily characterized by its base sequence. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term ‘homology’ denotes at least secondary structural identity or similarity between two macromolecules, particularly between two polypeptides or polynucleotides, from same or different taxons, wherein said similarity is due to shared ancestry. Hence, the term ‘homologues’ denotes so-related macromolecules having said secondary and optionally tertiary structural similarity. For comparing two or more nucleotide sequences, the ‘(percentage of) sequence identity’ between a first nucleotide sequence and a second nucleotide sequence may be calculated using methods known by the person skilled in the art, e.g. by dividing the number of nucleotides in the first nucleotide sequence that are identical to the nucleotides at the corresponding positions in the second nucleotide sequence by the total number of nucleotides in the first nucleotide sequence and multiplying by 100% or by using a known computer algorithm for sequence alignment such as NCBI Blast. In determining the degree of sequence similarity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions will be clear to the person skilled in the art. Amino acid sequences and nucleic acid sequences are said to be ‘exactly the same’ if they have 100% sequence identity over their entire length.

Throughout this application, each time one refers to a specific amino acid sequence SEQ ID NO (take SEQ ID NO: Y as example), one may replace it by: a polypeptide comprising an amino acid sequence that has at least 60% sequence identity or similarity with amino acid sequence SEQ ID NO: Y.

Each amino acid sequence described herein by virtue of its identity or similarity percentage (at least 60%) with a given amino acid sequence respectively has in a further preferred embodiment an identity or a similarity of at least 60%, 65%, 70%, 75%, 80%, 85%, 90%, 95%, 97%, 98%, 99% or more identity or similarity with the given amino acid sequence respectively. In a preferred embodiment, sequence identity or similarity is determined by comparing the whole length of the sequences as identified herein. Unless otherwise indicated herein, identity or similarity with a given SEQ ID NO means identity or similarity based on the full length of said sequence (i.e., over its whole length or as a whole).

“Sequence identity” is herein defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include, e.g., the GCG program package (Devereux, J., et al., Nucleic Acids Research 12 (1): 387 (1984)), BestFit, FASTA, BLASTN, and BLASTP (Altschul, S. F. et al., J. Mol. Biol. 215:403-410 (1990)), EMBOSS Needle (Madeira, F., et al., Nucleic Acids Research 47(W1): W636-W641 (2019)). The BLAST program is publicly available from NCBI and other sources (BLAST Manual, Altschul, S., et al., NCBI NLM NIH Bethesda, MD 20894; Altschul, S., et al., J. Mol. Biol. 215:403-410 (1990)). The EMOSS program is publicly available from EMBL-EBI. The well-known Smith Waterman algorithm may also be used to determine identity. The EMBOSS Needle program is the preferred program used.

Preferred parameters for polypeptide sequence comparison include the following: Algorithm: Needleman and Wunsch, J. Mol. Biol. 48 (3):443-453 (1970); Comparison matrix: BLOSUM62 from Henikoff and Henikoff, Proc. Natl. Acad. Sci. USA. 89:10915-10919 (1992); Gap Open Penalty: 10; and Gap Extend Penalty: 0.5. A program useful with these parameters is publicly available as the EMBOSS Needle program from EMBL-EBI. The aforementioned parameters are the default parameters for a Global Pairwise Sequence alignment of proteins (along with no penalty for end gaps).

Preferred parameters for nucleic acid comparison include the following: Algorithm: Needleman and Wunsch, J. Mol. Biol. 48:443-453 (1970); Comparison matrix: DNAfull; Gap Open Penalty: 10; Gap Extend Penalty: 0.5. A program useful with these parameters is publicly available as the EMBOSS Needle program from EMBL-EBI. The aforementioned parameters are the default parameters for a Global Pairwise Sequence alignment of nucleotide sequences (along with no penalty for end gaps).

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; a group of amino acids having acidic side chains is aspartate and glutamate; and a group of amino acids having sulphur-containing side chains is cysteine and methionine. Preferred conservative substitutions for each of the naturally occurring amino acids are as follows: Ala to Ser; Arg to Lys or Gln; Asn to Asp, His or Ser; Asp to Glu or Asn; Gln to Glu, Lys or Arg; Glu to Lys, Asp, Gln; His to Tyr or Asn; Ile to Leu, Val, or Met; Leu to Ile, Met or Val; Lys to Arg, Gln or Glu; Met to Val, Leu or Ile; Phe to Trp or Tyr; Ser to Thr, Ala or Asn; Thr to Ser; Trp to Tyr or Phe; Tyr to His, Trp or Phe; and Val to Ile, Leu or Met. Substitutional variants of the amino acid sequence disclosed herein are those in which at least one residue in the disclosed sequences has been removed and a different residue inserted in its place. Preferably, the amino acid change is conservative.

### Method for Isolating Suitable Antibody Fragment Against Human Folr1

In particular embodiments, antibody fragment, preferably VHH or fragment thereof disclosed herein are obtained by affinity selection against human FOLR1 present on and/or specific for a solid tumor and/or a cancer cell. Obtaining suitable polypeptides by affinity selection against a particular solid tumor antigen or cancer cell may for example be performed by screening a set, collection or library of cells that express antibody fragment, preferably VHH’s on their surface (e.g. bacteriophages) for binding against a tumor-specific antigen and/or a cancer cell-specific antigen; all of which may be performed in a manner known per se, essentially comprising the following non-limiting steps: a) obtaining an isolated solution or suspension of a tumor-specific or cancer cell-specific protein target molecule, which molecule is known to be a target for a potential cancer drug; b) bio-panning phages or other cells from a VHH library against said protein target molecule; c) isolating the phages or other cells binding to the tumor-specific or cancer cell-specific protein target molecule; d) determining the nucleotide sequence encoding the VHH insert from individual binding phages or other cells; e) producing an amount of VHH according to this sequence using recombinant protein expression and f) determining the affinity of said VHH domain for said tumor-specific or cancer cell-specific protein target molecule and optionally g) testing the tumoricidal or anticancer activity of said VHH domain in a bio-assay. Various methods may be used to determine the affinity between the VHH domain and the tumor-specific or cancer cell-specific protein target molecule, including for example, enzyme linked immunosorbent assays (ELISA) or Surface Plasmon Resonance (SPR) assays, which are common practice in the art, for example, as described in Sambrook et al. (2001), Molecular Cloning, A Laboratory Manual. Third Edition. Cold Spring Harbor Laboratory Press, Cold Spring Harbor, NY. The equilibrium dissociation constant is commonly used to describe the affinity between a polypeptide and its target molecule. Typically, the equilibrium dissociation constant is lower than 10⁻⁷ M. Preferably, the equilibrium dissociation constant is lower than 10⁻⁸ M, or lower than 10⁻⁹ M, or more preferably, ranged from 10⁻⁹ M and 10⁻¹¹ M.

### Antibody

As used herein, the term ‘antibody’ refers to polyclonal antibodies, monoclonal antibodies, humanized antibodies, single-chain antibodies, and fragments thereof such as Fab F(ab)2, Fv, VHH and other fragments that retain the antigen binding function of the parent antibody. As such, an antibody may refer to an immunoglobulin or glycoprotein, or fragment or portion thereof, or to a construct comprising an antigen-binding portion comprised within a modified immunoglobulin-like framework, or to an antigen-binding portion comprised within a construct comprising a non-immunoglobulin-like framework or scaffold.

As used herein, the term ‘monoclonal antibody’ refers to an antibody composition having a homogeneous antibody population. The term is not limited regarding the species or source of the antibody, nor is it intended to be limited by the manner in which it is made. The term encompasses whole immunoglobulins as well as fragments and others that retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies.

As used herein, the term ‘polyclonal antibody’ refers to an antibody composition having a heterogeneous antibody population. Polyclonal antibodies are often derived from the pooled serum from immunized animals or from selected humans.

‘Heavy chain variable domain of an antibody or a fragment thereof’, as used herein, means (i) the variable domain of the heavy chain of a heavy chain antibody, which is naturally devoid of light chains (also indicated hereafter as VHH), including but not limited to the variable domain of the heavy chain of heavy-chain antibodies of camelids or sharks or (ii) the variable domain of the heavy chain of a conventional four-chain antibody (also indicated hereafter as V_{H}), including but not limited to a camelized (as further defined herein) variable domain of the heavy chain of a conventional four-chain antibody (also indicated hereafter as camelized V_{H}) or any fragments thereof, such as but not limited to one or more stretches of amino acid residues (i.e. small peptides) that are particularly suited for binding to a tumor antigen or an antigen present on cancer cells and which are present in, and/or may be incorporated into, the VHH’s as disclosed herein (or may be based on and/or derived from CDR sequences of the VHH’s as disclosed herein). In an embodiment, the fragment of a VHH is a functional fragment.

As further described herein below, the amino acid sequence and structure of a heavy chain variable domain of an antibody can be considered, without however being limited thereto, to be comprised of four framework regions or ‘FR’s’, which are referred to in the art and herein below as ‘framework region 1’ or ‘FR1’; as ‘framework region 2’ or ‘FR2’; as ‘framework region 3’ or ‘FR3’; and as ‘framework region 4’ or ‘FR4’, respectively, which framework regions are interrupted by three complementary determining regions or ‘CDR’s’, which are referred to in the art as ‘complementarity determining region 1’ or ‘CDR1’; as ‘complementarity determining region 2’ or ‘CDR2’; and as ‘complementarity determining region 3’ or ‘CDR3’, respectively.

As used herein, the terms ‘complementarity determining region’ or ‘CDR’ within the context of antibodies refer to variable regions of either the H (heavy) or the L (light) chains (also abbreviated as VH and VL, respectively) and contain the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains.

As also further described herein below, the total number of amino acid residues in a heavy chain variable domain of an antibody (including a VHH or a V_{H}) can be in the region of 110-130. It should however be noted that parts, fragments or analogs of a heavy chain variable domain of an antibody are not particularly limited as to their length and/or size, as long as such parts, fragments or analogs retain (at least part of) the functional activity, and/or retain (at least part of) the binding specificity of the original heavy chain variable domain of an antibody from which these parts, fragments or analogs are derived from. Parts, fragments or analogs retaining (at least part of) the functional activity, and/or retaining (at least part of) the binding specificity of the original heavy chain variable domain of an antibody from which these parts, fragments or analogs are derived from are also further referred to herein as ‘functional fragments’ of a heavy chain variable domain.

The amino acid residues of a variable domain of an antibody (including a VHH or a V_{H}) are preferably numbered according to the IMGT unique numbering for V-domain (immunoglobulins and T cell receptors) given by the IMGT nomenclature as described (Lefranc M.P. et al 1997 Immunology today, 18: 509, PMID: 9386342; Lefranc, M.-P., 1999 The Immunologist, 7: 132-136 and Lefranc M.P. et al 2003, Dev. Comp. Immunol., 27: 55-77 PMID: 12477501). According to this numbering (see for example table 1 of Lefranc 2003), the conserved amino acids always have the same position, for instance cysteine 23 (1st-CYS), tryptophan 41 (CONSERVED-TRP), hydrophobic amino acid 89, cysteine 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. Gaps represent unoccupied positions. Gaps in the CDR1-IMGT and CDR2-IMGT (less than 12 and 10 amino acid long, respectively) are put at the top of the CDR-IMGT loops. The basic length of a rearranged CDR3-IMGT is 13 amino acids (positions 105 to 117), which corresponds to a JUNCTION of 15 amino acids (2nd-CYS 104 to J-TRP or J-PHE 118). If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1,111.1, 112.2, 111.2, 112.3, 111.3, etc.

In this respect, it should be noted that-as is well known in the art for VHH domains-the total number of amino acid residues in each of the CDR’s may vary and may not correspond to the total number of amino acid residues indicated by the IMGT numbering (that is, one or more positions according to the IMGT numbering may not be occupied in the actual sequence, or the actual sequence may contain more amino acid residues than the number allowed for by the IMGT numbering). This means that, generally, the numbering according to IMGT may or may not correspond to the actual numbering of the amino acid residues in the actual sequence.

Alternatively, the amino acid residues of a variable domain of an antibody (including a VHH or a VH) can be numbered according to Kabat numbering (Kabat et al 1987, National Institute of Health; 1987. 804 pp., Publication no. 165-462.). Correspondence between the IMGT and Kabat numbering for the immunoglobulin V-regions can be found for example in Table 2 of Lefranc et al., 2003.

For a general description of heavy chain antibodies and the variable domains thereof, reference is inter alia made to Muyldermans S., et al 2013 Annual Review of Biochemistry, 82: 775-797 as general background art.

Generally, it should be noted that the term ‘heavy chain variable domain’ as used herein in its broadest sense is not limited to a specific biological source or to a specific method of preparation. For example, as will be discussed in more detail below, the heavy chain variable domains derived from heavy chain antibodies (i.e. VHH’s) as disclosed herein can be obtained (1) by isolating the VHH domain of a naturally occurring heavy chain antibody; (2) by expression of a nucleotide sequence encoding a naturally occurring VHH domain; (3) by ‘camelization’ (as described below) of a naturally occurring V_{H} domain from any animal species, in particular a species of mammal, such as from a human being, or by expression of a nucleic acid encoding such a camelized V_{H} domain; (4) by ‘camelisation’ of a ‘domain antibody’ or ‘dAb’ as described by Weizao C., et al Methods Mol Biol 2009, 525:81-99)), or by expression of a nucleic acid encoding such a camelized V_{H} domain (5) using synthetic or semi-synthetic techniques for preparing proteins, polypeptides or other amino acid sequences; (6) by preparing a nucleic acid encoding a VHH using techniques for nucleic acid synthesis, followed by expression of the nucleic acid thus obtained; and/or (7) by any combination of the foregoing. Suitable methods and techniques for performing the foregoing will be clear to the skilled person based on the disclosure herein and for example include the methods and techniques described in more detail herein below.

An antibody fragment such as a single-domain antibody fragment as disclosed herein is considered to be ‘(in) essentially isolated (form)’ as used herein, when it has been extracted or purified from the host cell and/or medium in which it is produced.

### Variant of Antibody Fragments

It should be noted that the invention is not limited as to the origin of the antibody fragment, preferably VHH sequences or fragments thereof of the invention (or of the nucleotide sequences of the invention used to express them). Furthermore, the present invention is also not limited as to the way that the antibody fragment, preferably VHH sequences or nucleotide sequences as disclosed herein have been generated or obtained. Thus, the amino acid sequences as disclosed herein may be synthetic or semi-synthetic amino acid sequences, polypeptides or proteins.

The present invention also encompasses parts, fragments, analogs, mutants, variants, and/or derivatives of the antibody fragment, preferably VHH specifically binding to human FOLR1 as disclosed herein and/or polypeptides comprising or essentially consisting of one or more of such parts, fragments, analogs, mutants, variants, and/or derivatives, as long as these parts, fragments, analogs, mutants, variants, and/or derivatives are suitable for the purposes envisaged herein: deliver to the CNS a molecule linked to it and suitable to be used in diagnostic and therapeutic applications when linked to a radionuclide. Such parts, fragments, analogs, mutants, variants, and/or derivatives according to the invention are still capable of specifically binding to human FOLR1 and not capable of specifically binding to murine FOLR1 and to human FOLR2 and to human FOLR3.

For example, the invention provides a number of stretches of amino acid residues (i.e. small peptides), also referred to herein as CDR sequences or part of the antibody fragment and identified as SEQ ID NO, such as sequences having at least 60% identity with at least one of SEQ ID NO: 2, 3, 4, 5, 6, 7, 8, 9, 10, 11, 12, 17, 18, 19, 25, 26, 27, 33, 34, 35, 41, 42, 43 of the antibody fragment, preferably VHH’s as disclosed herein, that are particularly suited for binding to human FOLR1. These stretches may be regarded as being functional fragments of the antibody fragment preferably VHH’s as disclosed herein and may be present in, and/or may be incorporated into any suitable scaffold (protein), such as but not limited to the VHH’s or compound or labelled compounds as disclosed herein, in particular in such a way that they form (part of) the antigen binding site of that suitable scaffold or VHH. It should however be noted that the invention in its broadest sense is not limited to a specific structural role or function that these stretches of amino acid residues may have in the scaffolds or antibody fragment, preferably VHH’s as disclosed herein, as long as these stretches of amino acid residues allow these scaffolds or antibody fragment (preferably VHH’s) as disclosed herein to specifically bind to human FOLR1.

### Further Posttranslational Structural Characterization of the Antibody Fragment

In certain aspects, the antibody fragment, preferably VHH domains or fragments thereof specifically binding to human FOLR1 as disclosed herein may be optionally linked to one or more further groups, moieties, or residues via one or more linkers. These one or more further groups, moieties or residues can serve for binding to other targets of interest. It should be clear that such further groups, residues, moieties and/or binding sites may or may not provide further functionality to the antibody fragment as disclosed herein and may or may not modify its properties as disclosed herein. Such groups, residues, moieties or binding units may also for example be chemical groups which can be biologically active.

These groups, moieties or residues are, in particular embodiments, linked N- or C-terminally to the heavy chain variable domain, in particularly C-terminally linked.

In particular embodiments, the antibody fragment, preferably VHH domains or fragments thereof specifically binding to human FOLR1 antigen as disclosed herein may also have been chemically modified. For example, such a modification may involve the introduction or linkage of one or more functional groups, residues or moieties into or onto the antibody fragment, preferably VHH domain. These groups, residues or moieties may confer one or more desired properties or functionalities to the antibody fragment, preferably VHH domain. Examples of such functional groups will be clear to the skilled person.

For example, the introduction or linkage of such functional groups to antibody fragment, preferably VHH domains or fragments thereof can result in an increase in their solubility and/or their stability, in a reduction of their toxicity, or in the elimination or attenuation of any undesirable side effects, and/or in other advantageous properties.

In particular embodiments, the one or more groups, residues, moieties are linked to the antibody fragment, preferably VHH domains or fragments thereof via one or more suitable linkers or spacers.

In cases where all of the two or more binding sites of an antibody fragment such as a VHH or fragments thereof, as disclosed herein are directed against or specifically bind to the same site, determinant, part, epitope, domain or stretch of amino acid residues of the human FOLR1, the antibody fragment as disclosed herein is said to be ‘bivalent’ (in the case of two binding sites on the single-domain antibody fragment) or multivalent (in the case of more than two binding sites on the single-domain antibody fragment), such as for example trivalent.

In an embodiment, the antibody fragment, preferably VHH or fragment thereof is present in a monovalent format.

As used herein, the term ‘monovalent’ when referring to an antibody fragment, such as a VHH or fragments thereof, denotes an antibody fragment in monomeric form. A monovalent antibody fragment contains only one binding site. In this context, the binding site of an antibody fragment, such as a VHH or fragments thereof, encompasses the one or more ‘complementarity determining regions’ or ‘CDRs’ and/or the one or more regions identified herein as having at least 60% identity with at least one of SEQ ID NO: 2, 3, 4, 5, 6, 7, 8, 9, 10, 11, 12, 17, 18, 19, 25, 26, 27, 33, 34, 35, 41, 42, 43 of an antibody fragment that are directed against or specifically bind to a particular site, determinant, part, epitope, domain or stretch of amino acid residues of human FOLR1.

In particularly preferred embodiments, the present invention provides an antibody fragment, preferably a VHH or a fragment thereof in its monomeric form, i.e., comprising only one VHH domain. The small size of such molecule is attractive for therapeutic/diagnostic applications. For specific application, such a small size may also be attractive if a high tissue penetration is needed in order to reach an optimal therapeutic effect.

In alternative embodiments, however the present invention also provides an antibody fragment, preferably a VHH or a fragment comprising two or more identical or different VHH domains resulting in a bivalent (or multivalent) or a bispecific or (multispecific) polypeptide.

While the antibody fragment, preferably a VHH or a fragment thereof may be present in its monomeric form, in particular alternative embodiments, two or more of the antibody fragments, preferably VHHs or fragments thereof may be linked to each other or may be interconnected. In particular embodiments, the two or more antibody fragments, preferably two or more VHHs or fragments thereof are linked to each other via one or more suitable linkers or spacers. Suitable spacers or linkers for use in the coupling of such antibody fragment, as disclosed herein will be clear to the skilled person and may generally be any linker or spacer used in the art to link peptides and/or proteins.

Some particularly suitable linkers or spacers include for example, but are not limited to, polypeptide linkers such as glycine linkers, serine linkers, mixed glycine/serine linkers, glycine- and serine-rich linkers or linkers composed of largely polar polypeptide fragments, or homo- or heterobifunctional chemical crosslinking compounds such as glutaraldehyde or, optionally PEG-spaced, maleimides or NHS esters.

For example, a polypeptide linker or spacer may be a suitable amino acid sequence having a length between 1 and 50 amino acids, such as between 1 and 30, and in particular between 1 and 10 amino acid residues. It should be clear that the length, the degree of flexibility and/or other properties of the linker(s) may have some influence on the properties of the antibody fragments, preferably VHHs or fragments thereof, including but not limited to the affinity, specificity or avidity for the tumor target or the target on a cancer cell or pharmacological behavior. It should be clear that when two or more linkers are used, these linkers may be the same or different. In the context and disclosure of the present invention, the person skilled in the art will be able to determine the optimal linkers for the purpose of coupling antibody fragments, preferably VHHs or fragments thereof as disclosed herein without any undue experimental burden.

As used herein, the term ‘untagged’ when referring to an antibody fragment, such as a VHH or functional fragments thereof, denotes an antibody fragment that contains no extraneous polypeptide sequences (e.g., contains only an antibody fragment, preferably a VHH sequence, or a fragment thereof, preferably linked to a medicament and/or labeled with a radioisotope as described herein). Exemplary extraneous polypeptide sequences include carboxy-terminal polypeptide tags, e.g., a His-tag, a cysteine-containing tag (e.g., a GGC-tag as described in Pruszyski et al 2013 Nucl Med Biol 40: 52-59), and/or a Myc-tag. A His-tag may contain 4, 5, 6, 7, 8, 9, 10 Histidines. In an embodiment, 6 Histidines are present.

Also in one embodiment, the one or more groups, residues or moieties that may be present do not induce multimerization such as dimerization of the antibody fragment, preferably VHH or functional fragments thereof as disclosed herein.

Therefore in an embodiment, an antibody fragment such as a VHH or a fragment thereof is devoid of a tag that induces multimerization such as dimerization, preferably devoid of a cysteine-containing tag, preferably a GGC-tag.

Therefore in an embodiment, an antibody fragment such as a VHH or a fragment thereof is devoid of a carboxy-terminal polypeptide tag, preferably it is untagged.

Advantageously, kidney retention was shown to be significantly reduced when using an antibody fragment without a carboxy-terminal polypeptide tag compared to a polypeptide tagged, such as His-tagged and Myc-His-tagged antibody fragment (D′Huyvetter et al. (2014), Theranostics. 4(7):708-20).

The term ‘bi-specific’ when referring to an antibody fragment, such as a VHH, as disclosed herein implies that either a) two or more of the binding sites of an antibody fragment as disclosed herein are directed against or specifically bind human FOLR1 but not to the same (i.e. to a different) site, determinant, part, epitope, domain or stretch of amino acid residues of human FOLR1, the antibody fragment as disclosed herein is said to be ‘bi-specific’ (in the case of two binding sites on the antibody fragment or multispecific (in the case of more than two binding sites on the antibody fragment) or b) two or more binding sites of an antibody fragment as disclosed herein are directed against or specifically bind to different target molecules of interest. The term ‘multispecific’ is used in the case that more than two binding sites are present on the antibody fragment as disclosed herein.

Accordingly, a ‘bispecific’ antibody fragment, such as a ‘bispecific’ VHH or a ‘multi-specific’ antibody fragment, such as a ‘multispecific’ VHH as used herein, shall have the meaning of an antibody fragment, such as a VHH, as disclosed herein comprising respectively two or at least two binding sites, wherein these two or more binding sites have a different binding specificity. Thus, an antibody fragment, such as a VHH, as disclosed herein is considered ‘bispecific’ or ‘multispecific’ if respectively two or more than two different binding regions exist in the same, monomeric antibody fragment.

The ‘half-life’ of an antibody fragment, in particular such as a VHH or fragments thereof, as disclosed herein can generally be defined as the time that is needed for the in vivo serum concentration of the antibody fragment, as disclosed herein to be reduced by 50%. The in vivo half-life of an antibody fragment, as disclosed herein can be determined in any manner known to the person skilled in the art, such as by pharmacokinetic analysis. As will be clear to the skilled person, the half-life can be expressed using parameters such as the t½-alpha, t½-beta and the area under the curve (AUC). An increased half-life in vivo is generally characterized by an increase in one or more and preferably in all three of the parameters t½-alpha, t½-beta and the area under the curve (AUC).

The term “lifetime extended” when referring to an antibody fragment, such as a VHH or fragments thereof as disclosed herein, is used to denote that the antibody fragment has been modified to extend the half-life of the antibody fragment. Strategies for extending the half-life of antibodies and antibody fragments are well-known in the art and include for example, but without limitation, linkage (chemically or otherwise) to one or more groups or moieties that extend the half-life, such as polyethylene glycol (PEG) or bovine serum albumin (BSA) or human serum albumin (HSA), antibody Fc fragments, or antigen-binding antibody fragments targeting serum proteins such as serum albumin.

Therefore, in an embodiment, the antibody fragment such as a VHH or a functional fragment thereof is non-lifetime extended.

### Nucleic Acid Molecule Encoding the Antibody Fragment

In a further aspect, the present invention provides nucleic acid molecules represented by nucleic acid sequences encoding the antibody fragment, preferably the VHH or suitable fragments thereof as defined herein.

In an embodiment, this nucleic acid molecule is represented by a nucleic acid sequence that comprises, consists of or essentially consists of a nucleic acid sequence having at least 60% identity with any of SEQ ID NO: 82, 83, 84, 85 or 86. Preferably, the identity is at least 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. The identity is usually assessed over the full length of said SEQ ID NO. However, it is not excluded the identity is assessed over a portion of said SEQ ID NO as defined herein.

These nucleic acid sequences can also be in the form of a vector or a genetic construct or polynucleotide. The nucleic acid sequences as disclosed herein may be synthetic or semi-synthetic sequences, nucleotide sequences that have been isolated from a library (and in particular, an expression library), nucleotide sequences that have been prepared by PCR using overlapping primers, or nucleotide sequences that have been prepared using techniques for DNA synthesis known per se.

TABLE 5

nucleid acid molecule encoding the antibody fragment

| VHH | SEQ ID NO: | DNA sequence | Translates to SEQ ID NO: |
|---|---|---|---|
| A1 | 82 | GATGTGCAGCTGGTGGAGTCTGGGGGAGGCTTGGTGCA GCCTGGGGGGTCTCTGAGACTCTCCTGTGCAGCCTCTG AAAGCATCTTCAGTCGCAATCACATGAGCTGGTACCGCC AGGCTCCAGGGAAGCAGCGCGAGTTGGCCGCGATTATT ACTAGTGATGGTAACACAAACTATCCAGACTCCGTGAAG GGCCGATTCACCATCTCCAGAGACAACGCTAAGAACAC GGTGTATCTGCAAATGAACAGCCTGAAACCTGAGGACAC GGCCGTCTATTACTGTAATACCTTCGGTCGTAGCGTAGC TGGTTTTTATGACTACTGGGGCCAGGGGACCCAGGTCA CCGTCTCCTCA | 8 |
| A2 | 83 | GATGTGCAGCTGGTGGAGTCTGGGGGAGGCTTGGTGCA GGCTGGGGGGTCTCTGAGACTCTCCTGTGCAGCCGATG AAATGAAATTCAGTAATTATGACTTGGCCTGGTACCGCC GGGCTCCAGGGAAGCAGCGCGAGTTGGCCGCAATTATT ACTAGTGGTGGTAGCACAAACTATCCGGACTCCGTGAA GGGCCGATTCACCATCTCCAGAGACAACGCCAAGAACA | 9 |
| | | CGGTGTATCTAGAAATGAACAGCCTGAAACCTGAGGACA CAGCCGTCTATTACTGCAATGCACTTCGACCCTGGCCTC AATTTAGCGACTACTGGGGCCAGGGGACCCAGGTCACC GTCTCCTCA | |
| A3 | 84 | GATGTGCAGCTGGTGGAGTCTGGGGGAGGCTTGGTGCA GGCTGGGGGGTCTCTGAGACTCTCCTGTGGAGTCTCTG GGAACATGTTCAGTATGAATCTCATGGGCTGGTACCGCC AACCTCCAGGGAAGGAGCGCGACTGGGTCGCAGTTATT ACTAAAGATGGGAGCACAAACTATGCAGACTCCGTGAAG GGCCGATTCACCATCTCCAGGGACAACGCCAAGAACAC GGTGTCTCTGCAAATGAACAGCCTGAAACCTGAGGACA CAGCCGTCTACTACTGTAATGTCCAGGATGGTAGTGGTT GGATTCGGGCGTGGGGCTGGGACTGGGGCCAGGGAAC CCAGGTCACCGTCTCCTCA | 24 |
| A4 | 85 | GATGTGCAGCTGGTGGAGTCTGGAGGAGGATTGGCCAG GGCTGGGGGCTCTCTGAGACTCTCCTGTGCAGCCTCTG GACGCGCCTTCAGTAGTACGTATGCCATGGGTTGGTTC CGCCAGGCTCCAGGGAAGGAGCGTGAGTTTGTAGCGGA TATTAATTGGAGTGAAGGTAGCCCGATGTATATAGATTC CGTGAAGGACCGATTCACCATCTCCAGAGACAACGCCA GAAATTCGCTGTATCTTCAGATGAACAGCCTGAAATCTG AGGACACGGCCGTTTATTACTGTGCAGCGCGGAGGTTC GGTCAGGGGTATGAGTACTGGGGCCAGGGGACCCAGG TCACCGTCTCCTCA | 32 |
| A5 | 86 | GATGTGCAGCTGGTGGAGTCTGGGGGAGGCTTGGTGCA GGCTGGGGGGTCTCTGAGACTCTCCTGTGGAGTCTCTG GGAACATCTTCAGTATGAATCTCATGGGCTGGTACCGCC AACCTCCAGGGAAGGAGCGCGAGTGGGTCGCACTAATT ACTAAAGATGGGAGCACAAATTATGAAGACTCCGTGAAG GGCCGATTCACCATCTCCAGGGACAACGCCAAGAACAC GGTGTCTCTGCAAATGAACAGCCTGAAACCTGAGGACA CAGCCGTCTACTACTGTAATGCCCAGGATGGTAGTGGCT GGATTCGGGCGTGGGGCTGGGACTGGGGCCAGGGAAC CCAGGTCACCGTCTCCTCA | 40 |

### Constructs, Vectors, Host Cells

The genetic constructs as disclosed herein may be DNA or RNA, and are preferably double-stranded DNA. The genetic constructs of the invention may also be in a form suitable for transformation of the intended host cell or host organism in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e., a vector that can provide for expression in vitro and/or in vivo (e.g. in a suitable host cell, host organism and/or expression system).

Accordingly, in another further aspect, the present invention also provides vectors comprising one or more nucleic acid sequences as disclosed herein.

In still a further aspect, the present invention provides hosts or host cells or cells that comprise and preferably express or are capable of expressing one or more nucleic acid sequences and therefore one or more amino acid sequences as disclosed herein. Suitable examples of hosts or host cells will be clear to the skilled person.

### Methods of Producing and Manufacturing Antibody Fragments

The invention further provides methods for preparing or generating the antibody fragment, in particular such as a VHH or fragments thereof, as well as methods for producing nucleic acids encoding these and host cells, products and compositions comprising these antibody fragments, in particular such as a VHH or fragments thereof. Some preferred but non-limiting examples of such methods will become clear from the further description herein.

As will be clear to the skilled person, one particularly useful method for preparing an antibody fragment, in particular such as a VHH or fragments thereof as disclosed herein generally comprises the steps of:
(a) expressing a nucleotide sequence encoding an antibody fragment, in particular such as a VHH or fragments thereof as disclosed herein and
(b) optionally isolating and/or purifying said antibody fragment.

The nucleic acid encoding the antibody fragment may be comprised in a vector or genetic construct.

In particular embodiments envisaged herein, the antibody fragment, in particular such as a VHH or fragments thereof can be obtained by methods which involve generating a random library of VHH sequences and screening this library for a VHH sequence capable of specifically binding to human FOLR1.

Accordingly, in particular embodiments, methods for preparing an antibody fragment, in particular such as a VHH or fragments thereof as disclosed herein comprise the steps of
a) providing a set, collection or library of amino acid sequences of VHH domains; and
b) screening said set, collection or library of amino acid sequences for amino acid sequences that can bind to and/or have affinity for human FOLR1. and
c) isolating the amino acid sequence(s) that can bind to and/or have affinity for human FOLR1.

In such a method, the set, collection or library of VHH sequences may be any suitable set, collection or library of amino acid sequences. For example, the set, collection or library of amino acid sequences may be a set, collection or library of immunoglobulin fragment sequences (as described herein), such as a naïve set, collection or library of immunoglobulin fragment sequences; a synthetic or semi-synthetic set, collection or library of immunoglobulin fragment sequences; and/or a set, collection or library of immunoglobulin fragment sequences that have been subjected to affinity maturation.

In particular embodiments of this method, the set, collection or library of VHH sequences may be an immune set, collection or library of immunoglobulin fragment sequences, for example derived from a mammal that has been suitably immunized with human FOLR1 or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of VHH sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art, for example on the basis of the further disclosure herein. Reference is also made to the review by Hoogenboom in Nature Biotechnology, 23, 9, 1105-1116 (2005).

In other embodiments, the methods for generating the antibody fragment, in particular such as a VHH or fragments thereof as disclosed herein comprise at least the steps of:
a) providing a collection or sample of cells expressing VHH domain amino acid sequences;
b) screening said collection or sample of cells for cells that express an amino acid sequence that can bind to and/or have affinity for human FOLR1; and
c) either (i) isolating said amino acid sequence; or (ii) isolating from said cell a nucleic acid sequence that encodes said amino acid sequence, followed by expressing said amino acid sequence.

The collection or sample of cells may for example be a collection or sample of B-cells. Also, in this method, the sample of cells may be derived from a mammal that has been suitably immunized with human FOLR1 or with a suitable antigenic determinant based thereon or derived therefrom, such as an antigenic part, fragment, region, domain, loop or other epitope thereof. In one particular embodiment, the antigenic determinant may be an extracellular part, region, domain, loop or other extracellular epitope(s).

In other embodiments, the method for generating an antibody fragment, in particular such as a VHH or fragments thereof as disclosed herein directed against human FOLR1 may comprise at least the steps of:
a) providing a set, collection or library of nucleic acid sequences encoding a VHH domain amino acid sequence;
b) screening said set, collection or library of nucleic acid sequences for nucleic acid sequences that encode an amino acid sequence that can bind to and/or has affinity for human FOLR1; and
c) isolating said nucleic acid sequence, followed by expressing said amino acid sequence.

In the above methods, the set, collection or library of nucleic acid sequences encoding amino acid sequences may for example be a set, collection or library of nucleic acid sequences encoding a naïve set, collection or library of immunoglobulin fragment sequences; a set, collection or library of nucleic acid sequences encoding a synthetic or semi-synthetic set, collection or library of immunoglobulin fragment sequences; and/or a set, collection or library of nucleic acid sequences encoding a set, collection or library of immunoglobulin fragment sequences that have been subjected to affinity maturation.

In particular, in such a method, the set, collection or library of nucleic acid sequences encodes a set, collection or library of an antibody fragment, in particular such as a VHH or fragments thereof as disclosed herein directed against human FOLR1 (as defined herein).

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to the review by Hoogenboom in Nature Biotechnology, 23, 9, 1105-1116 (2005).

The invention also relates to an antibody fragment, in particular such as a VHH or fragments thereof as disclosed herein that are obtainable or obtained by the above methods, or alternatively by a method that comprises one of the above methods and in addition at least the steps of determining the nucleotide sequence or amino acid sequence of said antibody fragment, in particular such as a VHH or fragments thereof as disclosed herein; and of expressing or synthesizing said antibody fragment, in particular such as a VHH or fragments thereof as disclosed herein in a manner known per se, such as by expression in a suitable host cell or host organism or by chemical synthesis.

In some cases, the methods for producing the antibody fragment, in particular such as a VHH or fragments thereof as disclosed herein binding specifically to human FOLR1 as envisaged herein may further comprise the step of isolating from the amino acid sequence library at least one antibody fragment, in particular such as a VHH or fragments thereof as disclosed herein having detectable binding affinity for, or detectable in vitro effect on human FOLR1.

These methods may further comprise the step of amplifying a sequence encoding at least one antibody fragment, in particular such as a VHH or fragments thereof as disclosed herein having detectable binding affinity for, or detectable in vitro effect on the activity of human FOLR1. For example, a phage clone displaying a particular amino acid sequence, obtained from a selection step of a method described herein, may be amplified by reinfection of a host bacteria and incubation in a growth medium.

In particular embodiments, these methods may encompass determining the sequence of the one or more amino acid sequences capable of binding to human FOLR1.

Where an antibody fragment, in particular such as a VHH or fragments thereof as disclosed herein, comprised in a set, collection or library of amino acid sequences, is displayed on a suitable cell or phage or particle, it is possible to isolate from said cell or phage or particle, the nucleotide sequence that encodes that amino acid sequence. In this way, the nucleotide sequence of the selected amino acid sequence library member(s) can be determined by a routine sequencing method.

In further particular embodiments, the methods for producing an antibody fragment, in particular such as a VHH or fragments thereof as envisaged herein comprise the step of expressing said nucleotide sequence(s) in a host organism under suitable conditions, so as to obtain the actual desired amino acid sequence. This step can be performed by methods known to the person skilled in the art.

In addition, the obtained antibody fragment, in particular such as a VHH or fragments thereof as disclosed herein having detectable binding affinity for, or detectable in vitro effect on the activity of human FOLR1, may be synthesized as soluble protein construct, optionally after their sequence has been identified.

For instance, the antibody fragment, in particular such as a VHH or fragments thereof as disclosed herein obtained, obtainable or selected by the above methods can be synthesized using recombinant or chemical synthesis methods known in the art. Also, the amino acid sequences obtained, obtainable or selected by the above methods can be produced by genetic engineering techniques. Thus, methods for synthesizing the antibody fragment, in particular such as a VHH or fragments thereof as disclosed herein obtained, obtainable or selected by the above methods may comprise transforming or infecting a host cell with a nucleic acid or a vector encoding an amino acid sequence having detectable binding affinity for, or detectable in vitro effect on the activity of human FOLR1. Accordingly, the antibody fragment, in particular such as a VHH or fragments thereof as disclosed herein having detectable binding affinity for, or detectable in vitro effect on the activity of human FOLR1 can be made by recombinant DNA methods. DNA encoding the amino acid sequences can be readily synthesized using conventional procedures. Once prepared, the DNA can be introduced into expression vectors, which can then be transformed or transfected into host cells such as E. coli or any suitable expression system, in order to obtain the expression of amino acid sequences in the recombinant host cells and/or in the medium in which these recombinant host cells reside.

It should be understood, as known by someone skilled in the art of protein expression and purification, that the antibody fragment, in particular such as a VHH or fragments thereof as disclosed herein produced from an expression vector using a suitable expression system may be tagged (typically at the N-terminal or C-terminal end of the amino acid sequence) with e.g. a His-tag or other sequence tag for easy purification.

Transformation or transfection of nucleic acids or vectors into host cells may be accomplished by a variety of means known to the person skilled in the art including calcium phosphate-DNA coprecipitation, DEAE-dextran-mediated transfection, polybrene-mediated transfection, electroporation, microinjection, liposome fusion, lipofection, protoplast fusion, retroviral infection, and biolistics.

Suitable host cells for the expression of the desired heavy chain variable domain sequences may be any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic plant.

Thus, the application also provides methods for the production of an antibody fragment, in particular such as a VHH or fragments thereof as disclosed herein having detectable binding affinity for, or detectable in vitro effect on the activity of human FOLR1 comprising transforming, transfecting or infecting a host cell with nucleic acid sequences or vectors encoding such antibody fragment, in particular such as a VHH or fragments thereof as disclosed herein and expressing their amino acid sequences under suitable conditions.

In yet another embodiment, the invention further provides methods for the manufacture (‘or the production of’ which is equivalent wording) a pharmaceutical composition as disclosed herein.

In particular embodiments, the invention provides methods for producing a pharmaceutical composition as disclosed herein, at least comprising the steps of:
obtaining at least one antibody fragment, in particular such as a VHH or fragments thereof as disclosed herein, which specifically binds to human FOLR1, and
formulating said antibody fragment, in particular such as a VHH or fragments thereof in a pharmaceutical composition.

In particular embodiments of these methods, the step of obtaining at least one antibody fragment, in particular such as a VHH or fragments thereof as disclosed herein, which specifically binds to human FOLR1 comprises:
(a) expressing a nucleotide sequence encoding an antibody fragment, in particular such as a VHH or fragments thereof as disclosed herein, which specifically binds to human FOLR1, and optionally
(b) isolating and/or purifying the antibody fragment, in particular such as a VHH or fragments thereof as disclosed herein.

In other particular embodiments of these methods, the step of obtaining at least one antibody fragment, in particular such as a VHH or fragments thereof as disclosed herein, which specifically binds to human FOLR1 comprises:
a) providing a set, collection or library of VHH domain sequences or fragments of VHH sequences;
b) screening said set, collection or library of VHH domain sequences or sequences of fragments thereof for sequences that specifically bind to and/or have affinity for human FOLR1, and optionally
c) isolating the VHH sequences or sequences of fragments thereof that specifically bind to and/or have affinity for human FOLR1.

### Process for Labeling the Antibody Fragment

There are various radiolabeling strategies available to incorporate a radionuclide into a protein. The choice of technique for a radiochemist depends primarily on the radionuclide used. The radioactive isotopes of iodine possess the ability to be directly integrated into a molecule by electrophilic substitution or indirectly via conjugation. Radioactive metals on the other hand are labeled via complexation with a chelating agent. Many metallic radionuclides possess the ability to form stable complexes with chelating agents, thus allowing for conjugation with a protein. Radiolabeling molecules with iodine nuclides is of great importance in pharmaceutical radiochemistry. There are over thirty different identified iodine isotopes, but only four are commonly used in radioiodine chemistry: ¹²³I, ¹²⁴I, ¹²⁵I and ¹³¹I.

The direct radioiodination of a protein is a key method for the synthesis of tumor-targeting or cancer cell-targeting radiopharmaceuticals. Generally, there are two basic approaches of protein radioiodination. The most straightforward approach is direct protein labeling using electrophilic substitution at tyrosine and histidine residues. The radioiodide is oxidized in situ creating the electrophile *I⁺. This is done using oxidizing agents like chloramine T, Iodogen® and N-halosuccinimides. The generated electrophile attacks the electron-rich aromatic ring of the amino acid tyrosine, forming a σ-complex. This substitution is performed at the tyrosine residue due to the electron donating hydroxyl group which stabilizes the σ-complex. As the labeling of proteins must take place under mild conditions, the attachment of iodine to the tyrosine is highly suitable.

This method is performed under mild conditions, which is optimal for the labeling of proteins. This is however only possible when the protein contains accessible tyrosine or histidine residues.

Indirect iodination of proteins via conjugation is a frequently used alternative method. In this approach iodine is incorporated by the application of prosthetic groups containing two functional groups to enable both radioiodination and incorporation to the protein. There are a variety of prosthetic groups used for radioiodination, but the most frequently used are N-succinimidyl 5-[*1]iodo-3-pyridinecarboxyl ([¹³¹I]SIPC) and N-succinimidyl-3-[*I]-iodobenzoate ([*I]SIB). Both active esters are conjugated to amino groups of the protein and exhibit a high in vivo stability.

Another prosthetic group for the acylation of aromatic groups is N-succinimidyl-4-guanidinomethyl-3-[I-131]iodobenzbate ([I-131]SGMIB).

In particular embodiments of the present invention, the labelled compounds as disclosed herein are labelled with lodine-131 using N-succinimidyl-4-guanidinomethyl-3-[I-131]iodobenzbate ([I-131]SGMIB) or suitable derivatives or variants thereof.

Detailed protocols for radiotherapy are readily available to the expert (Cancer Radiotherapy: Methods and Protocols (Methods in Molecular Medicine), Huddart RA Ed., Human Press 2002). The skilled person knows how to determine an appropriate dosing and application schedule, depending on the nature of the disease and the constitution of the patient. In particular, the skilled person knows how to assess dose-limiting toxicity (DLT) and how to determine the maximum tolerated dose (MTD) accordingly.

In particular embodiments, the labelled compounds thereof as disclosed herein are administered at a radioactive dosage of lower than about 800 mCi, such as for instance lower than about 150 mCi, such as for instance lower than about 30 mCi, such as lower than about 15 mCi.

In particular embodiments, the radioimmunoconjugate has a specific activity of from about 0.5 mCi/mg to about 8000 mCi/mg, such as for instance from 1 mCi/mg to about 1500 mCi/mg, such as for instance from 1 mCi/mg to about 300 mCi/mg, such as for instance from 1 mCi/mg to about 150 mCi/mg, depending on the radionuclide, and may be administered via an intravenous, intraperitoneal or other route such as intrathecal route. Depending on the desired duration and effectiveness of the treatment, the labelled compounds as disclosed herein may be administered once or several times, in combination with other therapeutic drugs or radio-sensitizing agents. The amount of the labelled compounds applied depends on the precise nature of the carcinoma. The dose of radioactivity per administration must be high enough to be effective, but must be below the dose limiting toxicity (DLT).

### Formulation/Uses in Therapy/Diagnostic

In yet a further aspect, compositions are provided comprising one or more antibody fragment, preferably VHH or fragments thereof disclosed herein and/or nucleic acid sequences as envisaged herein and optionally at least one acceptable carrier.

According to certain particular embodiments, the compositions as envisaged herein may further optionally comprise at least one other compound.

As used herein, a “screening dose” or a “biomarker dose” is a dose of an agent, such as a labelled compound as described herein, that is sufficient for selecting a subject for treatment, such as a dose that can bind to a cancer cell or solid tumor in the subject and subsequently be detected at the location of the cancer cell or solid tumor, e.g., by imaging the subject using gamma camera imaging such as planar gamma camera imaging, single photon emission computed tomography or positron emission tomography, optionally combined with a non-nuclear imaging technique such as X-ray imaging, computed tomography and/or magnetic resonance imaging. In some embodiments, a screening dose is a dose that is not therapeutically effective. In some embodiments, the screening dose is different than (e.g., lower than) a therapeutic dose as described herein.

As used herein, a “therapeutic dose” is a dose of an agent, such as a labelled compound as described herein, that is therapeutically effective in at least 1%, at least 5%, at least 10%, at least 20%, at least 30%, at least 40%, or at least 50% of subjects in need of such treatment (e.g., in subjects having cancer). In some embodiments, the therapeutic dose is higher than a screening dose as described herein.

As used herein, “imaging a subject” refers to capturing one or more images of a subject using a device that is capable of detecting a labelled compound as described herein. The one or more images may be further altered by a computer program and/or a person skilled in the art in order to enhance the images (e.g., by adjusting contrast or brightness of the one or more images). Any device capable of detecting a labelled compound as described herein is contemplated for use, such as a device for gamma camera imaging such as planar gamma camera imaging, for single photon emission computed tomography or for positron emission tomography, or a device able to combine a nuclear imaging technique with a non-nuclear imaging technique such as X-ray imaging, computed tomography and/or magnetic resonance imaging. For example, such device can be a device for single photon emission computed tomography/computed tomography (SPECT/CT) or positron emission computed tomography/computed tomography (PET/CT) imaging. Such devices are known in the art and commercially available.

In some embodiments, the administration of the screening dose and the detection by imaging are separated by at least 1 about minute, at least 5 about minutes, at least about 10 minutes, at least about 20 minutes, at least about 30 minutes, at least about 40 minutes, at least about 50 minutes, at least about 1 hour, at least about 1.5 hours, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 24 hours, at least about 2 days, or at least about 7 days. In some embodiments, the administration of the screening dose and the detection are separated by between about 1 hour and about 24 hours.

In some embodiments, the screening dose and the therapeutic dose are administered at least about 1 day, at least about 2 days, at least about 3 days, at least about 4 days, at least about 5 days, at least about 6 days, at least about 1 week, at least about 2 weeks, at least about 3 weeks, at least one month, at least about 2 months, or at least about 6 months apart. In some embodiments, the screening dose and the therapeutic dose are administered between about 1 day and about 6 months apart (e.g., between about 1 day and about 2 months, between about 1 day and about 1 month, or between about 1 day and about 1 week apart).

The screening dose and therapeutic dose may each independently be administered by any suitable route, such as systemically, locally or topically. Exemplary routes include intravenous, intraperitoneal, and intrathecal administration. The particular route utilized may, in some embodiments, depend on the nature of the disease (e.g., type, grade, location and stage of the tumor or cancer cell etc.) and the type of subject (e.g., species, constitution, age, gender, weight, etc.).

As used herein for all diagnostic and therapeutic applications, the term “subject” generally refers to a mammal, such as a human, a non-human primate, a rat, a mouse, a rabbit, a dog, a cat, a pig, a horse, a goat, or a sheep. In some embodiments, the subject is a human subject. In some embodiments, the subject is a subject having cancer (e.g., a human subject having cancer). Methods for identifying subjects having cancer include detection of tumor antigens or other tumor biomarkers, genetic testing, MRI, X-ray, PET scan, biopsies, and combinations thereof.

As used herein, the terms ‘diagnosis’, ‘prediction’ and/or ‘prognosis’ as used herein comprise diagnosing, predicting and/or prognosing a certain disease and/or disorder and/or condition, thereby predicting the onset and/or presence of a certain disease and/or disorder and/or condition, and/or predicting the progress and/or duration of a certain disease and/or disorder and/or condition, and/or predicting the response of a patient suffering from of a certain disease and/or disorder and/or condition to therapy.

In some embodiments of any one of the labelled compounds, composition comprising the same or diagnostic or therapeutic applications provided, a screening dose (i.e. used in a diagnostic method) is a dose that is not therapeutically effective. In some embodiments, the screening dose is lower than a therapeutic dose as described herein (e.g., at least about 10 times, at least about 20 times, at least about 30 times, at least about 40 times, at least about 50 times, at least about 100 times, at least about 200 times, at least about 300 times, at least about 400 times, at least about 500 times or at least about 1000 times lower than a therapeutic dose as described herein, or at least about 10, at least about 20, at least about 30, at least about 40, at least about 50, at least about 60, at least about 70, at least about 80, at least about 90, at least about 100, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 320, at least about 340, at least about 360, at least about 380, at least about 400, at least about 450, at least about 500, at least about 1000, at least about 5000, at least about 10000, at least about 13000, at least about 15000, at least about 18000, or at least about 20000 MBq lower than a therapeutic dose as described herein). In some embodiments, the screening dose is between 10 about MBq and about 400 MBq, between about 20 MBq and about 400 MBq, between about 30 MBq and about 400 MBq, about 40 MBq and about 400 MBq, between about 50 MBq and about 400 MBq, between about 100 MBq and about 400 MBq, between about 200 MBq and about 400 MBq, between about 300 MBq and about 400 MBq, between about 10 MBq and about 300 MBq, between about 20 MBq and about 300 MBq, between about 30 MBq and about 300 MBq, about 40 MBq and about 300 MBq, between about 50 MBq and about 300 MBq, between about 100 MBq and about 300 MBq, or between about 200 MBq and about 300 MBq. In some embodiments, the screening dose is between 3 about 7 MBq and about 370 MBq. It is to be understood that any screening dose described herein may be combined with any therapeutic dose as described herein.

In some embodiments, the therapeutic dose is higher than a screening dose as described herein (e.g., at least about 10 times, at least about 20 times, at least about 30 times, at least about 40 times, at least about 50 times, at least about 100 times, at least about 200 times, at least about 300 times, at least about 400 times, at least about 500 times or at least about 1000 times higher than a screening dose as described herein, or at least about 10, at least about 20, at least about 30, at least about 40, at least about 50, at least about 60, at least about 70, at least about 80, at least about 90, at least about 100, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 320, at least about 340, at least about 360, at least about 380, at least about 400, at least about 450, at least about 500, at least about 1000, at least about 5000, at least about 10000, at least about 13000, at least about 15000, at least about 18000, or at least about 20000 MBq higher than a screening dose as described herein). In some embodiments, the therapeutic dose is between about 300 MBq and about 20000 MBq, between about 400 MBq and about 20000 MBq, between about 500 MBq and about 20000 MBq, between about 1000 MBq and about 20000 MBq, between about 2000 MBq and about 20000 MBq, between about 3000 MBq and about 20000 MBq, between about 4000 MBq and about 20000 MBq, between about 5000 MBq and about 20000 MBq, between about 10000 MBq and about 20000 MBq, between about 5000 MBq and about 20000 MBq, between about 10000 MBq and about 20000 MBq, between about 300 MBq and about 10000 MBq, between about 400 MBq and about 10000 MBq, between about 500 MBq and about 10000 MBq, between about 1000 MBq and about 10000 MBq, between about 2000 MBq and about 10000 MBq, between about 3000 MBq and about 10000 MBq, between about 4000 MBq and about 10000 MBq, or between about 5000 MBq and about 10000 MBq. In some embodiments of any one of the methods provided, the therapeutic dose is between about 370 MBq and about 18500 MBq.

The screening and/or therapeutic dose may conveniently be presented in a single dose or as divided doses (which can again be sub-dosed) administered at appropriate intervals. An administration regimen of the therapeutic dose could include long-term (e.g., at least two weeks, and for example several months or years) or daily treatment. In some embodiments, an administration regimen of the therapeutic dose can vary between once a day to once a month, such as between once a day and once every two weeks, such as but not limited to once a week. Thus, in some embodiments, pharmaceutical compositions as disclosed herein may be administered once or several times, also intermittently, for instance on a daily basis for several days, weeks or months.

The particular screening dose and therapeutic dose utilized may, in some embodiments, depend on the nature of the disease (e.g., type, grade, and stage of the tumor or cancer cell etc.) and the type of subject (e.g., species, constitution, age, gender, weight, etc.).

In some aspects, the invention provides kits, such as a kit for diagnostic and therapeutic applications as described herein. In some embodiments, the kit comprises a screening dose of a labelled compound as described herein and a therapeutic dose of the same compound. Screening doses and therapeutic doses are described herein.

In some embodiments of any one of the kits, the kit further comprises one or more means for injection of the screening dose and the therapeutic dose. In some embodiments, the screening dose and therapeutic dose are each individually housed in a means for injection. In some embodiments, the means for injection is a syringe. In some embodiments of any one of the kits, the kit further comprises instructions for carrying out a method as described herein (e.g., a method of stratifying and treating a subject as described herein). The instructions may be in any suitable form, e.g., in printed form (e.g., as a paper or laminated insert or label) or in electronic form (e.g., on a disc or USB stick).

Dose, route of administration, application scheme, repetition and duration of treatment will in general depend on the nature of the disease (type, grade, and stage of the tumor or cancer cell or type, grade and stage of the disease or condition associated with the CNS or the brain etc.) and the patient (constitution, age, gender etc.), and will be determined by the skilled medical expert responsible for the treatment. With respect to the possible doses for the components of the disclosed combination which are described above, it is clear that the medical expert responsible for the treatment will carefully monitor whether any dose-limiting toxicity or other severe side effects occur and undertake the necessary steps to manage those.

Generally, for pharmaceutical (diagnostic and therapeutic) use, the (labelled) compound comprising an antibody fragment, preferably VHH or fragments thereof as envisaged herein may be formulated as a pharmaceutical preparation or compositions comprising the (labelled) compound as envisaged herein and at least one pharmaceutically acceptable carrier, diluent or excipient and/or adjuvant, and optionally one or more further pharmaceutically active polypeptides and/or compounds. Such a labelled compound or composition comprising the same may be suitable for intraperitoneal, intravenous or other administration such as intrathecal administration. Thus, the (labelled) compounds and/or the compositions comprising the same can for example be administered systemically, locally or topically to the tissue or organ of interest, depending on the location, type and origin of the tumor or cancer cell, and preferably intraperitoneally, intravenously or intrathecally, depending on the specific pharmaceutical formulation or composition to be used. The clinician will be able to select a suitable route of administration and a suitable pharmaceutical formulation or composition to be used in such administration. The same holds for the compound which is to deliver a medicament to the CNS, preferably the brain.

The pharmaceutical dosage forms suitable for injection or infusion can include sterile aqueous solutions or dispersions or sterile powders comprising the active ingredients which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

The amount of the (labelled) compound as envisaged herein required for use in prophylaxis and/or treatment may vary not only with the particular antibody fragment, preferably a VHH or functional fragments thereof but also with the route of administration, the nature of the condition being treated and the age and condition of the patient and will be ultimately at the discretion of the attendant physician or clinician. Also, the dosage of the (labelled) compound envisaged herein may vary depending on the target cell, tumor, tissue, graft, or organ.

In particular, the (labelled) compound as envisaged herein will be administered in an amount which will be determined by the medical practitioner based inter alia on the severity of the condition and the patient to be treated. Typically, for each disease indication an optimal dosage will be determined specifying the amount to be administered per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment.

Useful dosages of the (labelled) compound thereof as envisaged herein can be determined by determining their in vitro activity, and/or in vivo activity in animal models. The non-human animal of the invention may be used to this end.

In certain embodiments, the present invention provides a labelled compound as disclosed herein for use in the prevention and/or treatment of cancer (preferably cancer which is associated with the expression of human FOLR1 on cancer cells) by administering to a subject in need thereof the labelled compound at a dose of ranged from 10 µg and 10 mg or from 10 µg and 7 mg or from 10 µg and 5 mg or from 10 µg and 2 mg or from 10 µg and 1.5 mg or from 10 µg and 1 mg of VHH. In further particular embodiments, the present invention provides a labelled compound as disclosed herein for use in the prevention and/or treatment of cancer by administering to a subject in need thereof the labelled compound at a dose ranged from 10 µg and 2 mg of labelled compound, such as in particular ranged from 10 µg and 1.5 mg or ranged from 100 µg and 1 mg of labelled compound.

Accordingly, the dose of radioactivity applied to the patient per administration has to be high enough to be effective but must be below the dose limiting toxicity (DLT). For pharmaceutical compositions comprising radiolabeled antibodies, e.g., with 131-lodine, the maximally tolerated dose (MTD) has to be determined which must not be exceeded in therapeutic settings.

The compound and labeled compound as envisaged herein and/or the compositions comprising the same are administered according to a regimen of treatment that is suitable for preventing and/or treating the disease or disorder to be prevented or treated. The clinician will generally be able to determine a suitable treatment regimen. Generally, the treatment regimen will comprise the administration of a labelled compound, or of one or more compositions comprising the same, in one or more pharmaceutically effective amounts or doses.

The desired dose may conveniently be presented in a single dose or as divided doses (which can again be sub-dosed) administered at appropriate intervals. An administration regimen could include long-term (i.e., at least two weeks, and for example several months or years) or daily treatment. In particular, an administration regimen can vary between once a day to once a month, such as between once a day and once every two weeks, such as but not limited to once a week. Thus, depending on the desired duration and effectiveness of the treatment, labelled compound or composition comprising the same as disclosed herein may be administered once or several times, also intermittently, for instance on a daily basis for several days, weeks or months and in different dosages. The amount applied of the labelled compound or composition disclosed herein depends on the nature of the particular cancer disease. Multiple administrations are preferred. However, radiolabelled materials are typically administered at intervals of 4 to 24 weeks apart, preferable 12 to 20 weeks apart. The skilled artisan knows however how to choose dividing the administration into two or more applications, which may be applied shortly after each other, or at some other predetermined interval ranging, e.g., from 1 day to 1 week.

In particular, the labelled compounds as envisaged herein may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician.

In the context of this invention, “in combination with”, “in combination therapy” or “in combination treatment” shall mean that the labelled compound as disclosed herein or composition comprising these labelled compounds as disclosed herein are applied together with one or more other pharmaceutically active compounds or principles to the patient in a regimen wherein the patient may profit from the beneficial effect of such a combination. In particular, both treatments are applied to the patient in temporal proximity. In a preferred embodiment, both treatments are applied to the patient within four weeks (28 days). More preferably, both treatments are applied within two weeks (14 days), more preferred within one week (7 days). In a preferred embodiment, the two treatments are applied within two or three days. In another preferred embodiment, the two treatments are applied at the same day, i.e., within 24 hours. In another embodiment, the two treatments are applied within four hours, or two hours, or within one hour. In another embodiment, the two treatments are applied in parallel, i.e., at the same time, or the two administrations are overlapping in time.

In particular non-limiting embodiments, the labelled compounds or composition comprising these labelled compounds as disclosed herein are applied together with a plasma or blood substitute such as modified gelatin. An example of modifed gelatin that may be used in this context is Gelofusine™. The use of such plasma or blood substitute is expected to optimize and therefore reduce the retention of the labeled compound in the kidney and therefore to optimize unwanted side effects.

In particular non-limiting embodiments, the labelled compounds or composition comprising these labelled compounds as disclosed herein are applied together with immunotherapy. In an embodiment, with one or more therapeutic antibodies or therapeutic antibody fragments. Thus, in these particular non-limiting embodiments, the radioimmunotherapy with the labelled compounds as disclosed herein or composition comprising these labeled compounds is combined with regular immunotherapy with one or more therapeutic antibodies or therapeutic antibody fragments. In further particular embodiments, the labelled compounds as disclosed herein or composition comprising these labeled compounds as disclosed herein are used in a combination therapy or a combination treatment method with one or more therapeutic antibodies or therapeutic antibody fragments.

In an embodiment, there is provided a combination therapy comprising a labelled compound as defined herein and an additional antibody or antibody fragment.

For example, the labelled compounds as disclosed herein or composition comprising these labeled compounds and the one or more therapeutic antibodies or therapeutic antibody fragments or plasma or blood substitutes may be infused at the same time, or the infusions may be overlapping in time. If the two drugs or component are administered at the same time, they may be formulated together in one single pharmaceutical preparation, or they may be mixed together immediately before administration from two different pharmaceutical preparations, for example by dissolving or diluting into one single infusion solution. In another embodiment, the two drugs or component are administered separately, i.e., as two independent pharmaceutical compositions. In one preferred embodiment, administration of the two treatments is in a way that tumour cells within the body of the patient are exposed to effective amounts of the cytotoxic drug and the radiation at the same time. In another preferred embodiment, effective amounts of both the labelled compounds as disclosed herein or composition comprising these labeled compounds as disclosed herein and the one or more therapeutic antibodies or therapeutic antibody fragments or component are present at the site of the tumour at the same time. The present invention also embraces the use of further agents, which are administered in addition to the combination as defined. This could be, for example, one or more further chemotherapeutic agent(s). It could also be one or more agent(s) applied to prevent, suppress, or ameliorate unwanted side effects of any of the other drugs given. For example, a cytokine stimulating proliferation of leukocytes may be applied to ameliorate the effects of leukopenia or neutropenia.

The efficacy of the compound or labelled compounds described herein, and of compositions comprising the same, can be tested using any suitable in vitro assay, cell-based assay, in vivo assay and/or animal model known per se, or any combination thereof, depending on the specific disease or disorder involved. Suitable assays and animal models will be clear to the skilled person. A suitable animal model is the non-human animal disclosed as an aspect of the invention and expressing human FOLR1.

The skilled person will generally be able to select a suitable in vitro or in vivo assay, cellular assay or animal model to test the antibody fragment, preferably VHH or fragments thereof or compound or labelled compound or composition comprising the same (all defined herein) for binding to human FOLR1; as well as for their therapeutic and/or prophylactic effect in respect of one or more cancer-related diseases and disorders or for their efficacy in delivering a medicament to the brain. Such assay may be an imaging assay as disclosed herein.

The term ‘effective amount’, as used herein, means the amount needed to achieve the desired result or results.

As used herein, the terms ‘determining’, ‘measuring’, ‘assessing’, ‘monitoring’ and ‘assaying’ are used interchangeably and include both quantitative and qualitative determinations.

As used herein, the term ‘prevention and/or treatment’ comprises preventing and/or treating a certain disease and/or disorder and/or condition, preventing the onset of a certain disease and/or disorder and/or condition, slowing down or reversing the progress of a certain disease and/or disorder and/or condition, preventing or slowing down the onset of one or more symptoms associated with a certain disease and/or disorder and/or condition, reducing and/or alleviating one or more symptoms associated with a certain disease and/or disorder and/or condition, reducing the severity and/or the duration of a certain disease and/or disorder and/or condition, and generally any prophylactic or therapeutic effect of the antibody fragment as disclosed herein that is beneficial to the subject or patient being treated.

### Cancer/Tumour/Metastatic Cell

As used herein, the term ‘tumor cell’ refers to a cell that is present in a primary or metastatic tumour lesion. In this context, tumours consist not only of cancer cells, but should be considered as organ-like structures in which a complex bidirectional interplay exists between transformed and non-transformed cells. The malignant potential of transformed cells requires an apt support structure from the stroma, which can consist of fibroblasts, adipocytes, blood and lymph vessels, but may also be considerably infiltrated by a wide range of immune cells.

By “solid tumor(s)” or “tumor(s)” are meant primary tumors and/or metastases (wherever located).

As used herein, the term ‘cancer cell’ refers to a cell that divides and reproduces abnormally and limitlessly with uncontrolled growth and which can break away and travel to other parts of the body and set up another site, referred to as metastasis.

A ‘lesion’ as used herein can refer to any abnormal change in a body tissue or organ resulting from injury or disease. In cancer terminology, lesion typically refers to a tumour.

The term ‘primary tumour(s)’ as used herein is a tumor growing at the anatomical site where tumor progression began and proceeded to yield a cancerous mass.

The term ‘metastatic lesion(s)’ as used herein refers to malignant, or cancerous, tumours that have spread from their original location to other parts of the body. Related medical terms that might be used interchangeably include late-stage cancer, advanced cancer, or metastatic disease. In general, metastatic lesions are considered to be incurable, although treatment is often available to control the spread of cancerous cells and potentially increase the individual’s life expectancy.

Metastasis is the term for the spread of cancer beyond its originating site in the body. Thus, metastatic lesions are cancerous tumours that are found in locations apart from the original starting point of the primary tumour. Metastatic tumours occur when cells from the primary tumour break off and travel to distant parts of the body via the lymph system and blood stream. Alternately, cells from the original tumour could seed into new tumours at adjacent organs or tissues. ‘Metastatic disease’ as used herein refers to late-stage cancer and to the medical classification of cancer as being in stage III, when cancer cells are found in lymph nodes near the original tumour, or in stage IV, when cancer cells have travelled far beyond the primary tumour site to distant parts of the body. Metastatic lesions are most commonly found in the brain, lungs, liver, or bones. An individual with metastatic cancer might or might not experience any symptoms, and the symptoms could be related to the area where metastasized cells have relocated. Once metastatic lesions are present in the body, the individual’s cancer will be considered incurable for most cancer types. This means it is excessively difficult to eradicate every existing cancer cell with available treatments. In this case, the goal of treatment becomes slowing the growth of tumours to maintain the highest possible quality of life and potentially extend the individual’s life expectancy. In some cases, people with metastatic lesions can live for a number of years with appropriate treatment for symptom management.

### Radiolabel/Label/ Radionuclide/Dose

As used herein, the term “labelled” as in “labelled compound” refers to the radioisotopic labeling of that antibody fragment or VHH or fragment thereof, wherein the antibody fragment or VHH or fragment thereof is labelled by including, coupling, or chemically linking a radionuclide to its amino acid sequence structure.

As used herein, the terms ‘radionuclide’, ‘radioactive nuclide’, ‘radioisotope’ or ‘radioactive isotope’, are used interchangeably herein and refer to atoms with an unstable nucleus, characterized by excess energy available to be imparted either to a newly created radiation particle within the nucleus or via internal conversion. During this process, the radionuclide is said to undergo radioactive decay, resulting in the emission of gamma ray(s) and/or subatomic particles such as alpha or beta particles. These emissions constitute ionizing radiation. Radionuclides occur naturally or can be produced artificially.

### IHC Techniques

The term ‘immunohistochemistry (IHC)’ as used herein refers to the process of detecting antigens (e.g., proteins) in cells of a tissue section by exploiting the principle of antibodies binding specifically to antigens in sections of biological tissues. Immunohistochemical staining is widely used in the diagnosis of abnormal cells such as those found in cancerous tumors. IHC is also widely used in basic research to understand the distribution and localization of biomarkers and differentially expressed proteins in different parts of a biological tissue.

TABLE 6

Sequence listing

| Seq id no | Amino acid/nucleic acid | Characterization of the sequence |
|---|---|---|
| 1 | Amino acid | Human FOLR1 |
| 2 | Amino acid | IMGT: part of A1: N37-W118 = longest region of A1 interacting with human FOLR1 |
| 3 | Amino acid | IMGT: part of A2: D38-W118 = longest region of A2 interacting with human FOLR1 |
| 4 | Amino acid | IMGT: 1^{st} of 2 discrete regions of A1 interacting with human FOLR1 = N37-D69 |
| 5 | Amino acid | IMGT: 2^{nd} of 2 discrete regions of A1 interacting with human FOLR1 = N105-W118 |
| 6 | Amino acid | IMGT: 1^{ST} of 2 discrete regions of A2 interacting with human FOLR1= D38-D69 |
| 7 | Amino acid | IMGT: 2^{nd} of 2 discrete regions of A2 interacting with human FOLR1= N105-W118 |
| 8 | Amino acid | Full length sequence of A1 |
| 9 | Amino acid | Full length sequence of A2 |
| 10 | Amino acid | CDR1 of A1 |
| 11 | Amino acid | CDR2 of A1 |
| 12 | Amino acid | CDR3 of A1 |
| 13 | Amino acid | FR1 of A1 |
| 14 | Amino acid | FR2 of A1 |
| 15 | Amino acid | FR3 of A1 |
| 16 | Amino acid | FR4 of A1 |
| 17 | Amino acid | CDR1 of A2 |
| 18 | Amino acid | CDR2 of A2 |
| 19 | Amino acid | CDR3 of A2 |
| 20 | Amino acid | FR1 of A2 |
| 21 | Amino acid | FR2 of A2 |
| 22 | Amino acid | FR3 of A2 |
| 23 | Amino acid | FR4 of A2 |
| 24 | Amino acid | Full length sequence of A3 |
| 25 | Amino acid | CDR1 of A3 |
| 26 | Amino acid | CDR2 of A3 |
| 27 | Amino acid | CDR3 of A3 |
| 28 | Amino acid | FR1 of A3 |
| 29 | Amino acid | FR2 of A3 |
| 30 | Amino acid | FR3 of A3 |
| 31 | Amino acid | FR4 of A3 |
| 32 | Amino acid | Full length sequence of A4 |
| 33 | Amino acid | CDR1 of A4 |
| 34 | Amino acid | CDR2 of A4 |
| 35 | Amino acid | CDR3 of A4 |
| 36 | Amino acid | FR1 of A4 |
| 37 | Amino acid | FR2 of A4 |
| 38 | Amino acid | FR3 of A4 |
| 39 | Amino acid | FR4 of A4 |
| 40 | Amino acid | Full length sequence of A5 |
| 41 | Amino acid | CDR1 of A5 |
| 42 | Amino acid | CDR2 of A5 |
| 43 | Amino acid | CDR3 of A5 |
| 44 | Amino acid | FR1 of A5 |
| 45 | Amino acid | FR2 of A5 |
| 46 | Amino acid | FR3 of A5 |
| 47 | Amino acid | FR4 of A5 |
| 48 | Amino acid | HA-tagged variant of A1 |
| 49 | Amino acid | HA-tagged variant of A2 |
| 50 | Amino acid | HA-tagged variant of A3 |
| 51 | Amino acid | HA-tagged variant of A4 |
| 52 | Amino acid | HA-tagged variant of A5 |
| 53 | Amino acid | His6-tagged variant of A1 |
| 54 | Amino acid | His6-tagged variant of A2 |
| 55 | Amino acid | His6-tagged variant of A3 |
| 56 | Amino acid | His6-tagged variant of A4 |
| 57 | Amino acid | His6-tagged variant of A5 |
| 58 | Nucleic acid | Human FOLR1 |
| 59 | Nucleic acid | Targeting vector to knock-in human folr1 cDNA after the stop-codon in exon 4 of the mouse folr1 gene, by homologous recombination |
| 60 | Amino Acid | Non-targeting control VHH R3B23 |
| 61 | Amino acid | Part of A3: N37-W118 (IMGT) |
| 62 | Amino acid | Part of A3: N37-D69 (IMGT) |
| 63 | Amino acid | Part of A3: N105-W118 (IMGT) |
| 64 | Amino acid | Part of A4: A38-W118 (IMGT) |
| 65 | Amino acid | Part of A4: A38-D69 (IMGT) |
| 66 | Amino acid | Part of A4: A105-W118 (IMGT) |
| 67 | Amino acid | Part of A5: N37-W118 (IMGT) |
| 68 | Amino acid | Part of A5: N37-D69 (IMGT) |
| 69 | Amino acid | Part of A5: N105-W118 (IMGT) |
| 70 | Amino acid | D59E variant of A1 |
| 71 | Amino acid | N64E variant of A1 |
| 72 | Amino acid | N64Q variant of A1 |
| 73 | Amino acid | N64S variant of A1 |
| 74 | Amino acid | E90Q variant of A2 |
| 75 | Amino acid | K29Q-E90Q variant of A2 |
| 76 | Amino acid | K29R-E90Q variant of A2 |
| 77 | Amino acid | M28A-E90Q variant of A2 |
| 78 | Amino acid | M28T-E90Q variant of A2 |
| 79 | Amino acid | HA-tag |
| 80 | Amino acid | Other HA-tag |
| 81 | Amino acid | Cys-tag |
| 82 | DNA | Sequence coding for A1 (i.e. SEQ ID NO:8) |
| 83 | DNA | Sequence coding for A2 (i.e. SEQ ID NO:9) |
| 84 | DNA | Sequence coding for A3 (i.e. SEQ ID NO:24) |
| 85 | DNA | Sequence coding for A4 (i.e. SEQ ID NO:32) |
| 86 | DNA | Sequence coding for A5 (i.e. SEQ ID NO:40) |

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

### EXAMPLES

### Example 1: Production of VHH A1, A2 and A3 and Variants Thereof

The hemagglutinin-tagged (HA-tagged) variants of VHHs A1, A2, A3, A4 and A5 (SEQ ID NO: 48, 49, 50, 51 and 52 resp.) were produced in E. coli TG1 cells transduced with the VHH phagemid vector and extracted from the periplasm by freeze-thaw methods, according to standard protocols such as those in Vincke, C., et al. (2012). Methods in Molecular Biology 907: 145-176. In short, bacteria were grown in 2xTY conditioned medium and VHH expression was induced in the exponential growth phase. During further growth VHHs were translocated to the periplasm, from which they were extracted by a freeze-thaw cycle.

The hexahistidine-tagged (His6-tagged) variants of VHHs A1, A2, A3, A4 and A5 (SEQ ID NO: 53, 54, 55, 56 and 57 resp.) were produced in E. coli WK6 cells transformed with the VHH recombinant expression plasmid and purified from the periplasm by immobilized-metal affinity chromatography (IMAC) and size-exclusion chromatography (SEC), according to standard protocols such as those in Vincke, C., et al. (2012). Methods in Molecular Biology 907: 145-176. In short, bacteria were grown in Terrific Broth conditioned medium and VHH expression was induced in the exponential growth phase. During overnight growth VHHs were translocated to the periplasm, from which they were collected with an osmotic shock. VHHs were purified from the periplasmic extract via IMAC by batch incubation with HIS-select suspension (Sigma-Aldrich) followed by a stepwise elution with 0.5 M imidazole. Subsequent SEC was performed on a HiLoad 16/600 Superdex 75 PG column (GE Healthcare) or Superdex 75 10/300 GL (GE Healthcare), equilibrated in PBS.

The untagged variants of VHHs A1, A2 and A3 (SEQ ID NO: 8, 9 and 24 resp.) were produced in HEK293-E cells transiently transfected with endotoxin-free plasmid DNA. Six days post transfection, conditioned medium containing the VHH was harvested by centrifugation (Baldi et al (2005) Biotechnol Prog. 21(1):148-53). VHHs were purified from the harvested medium via protein A-affinity chromatography by batch incubation with rmp Protein A Sepharose (GE Healthcare) followed by a stepwise acidic elution with 20 mM citrate, 150 mM NaCl, pH 3.0 and neutralization of the eluate fractions with 1 M K2HPO4/KH2PO4, pH 8.0 to reach a final pH of 7.0. Alternatively, VHHs were purified from the harvested medium via cation-exchange chromatography using a Capto SP ImpRes column (GE Healthcare) equilibrated in 20 mM NaAc, pH 4.0 and employing a salt gradient. Subsequent SEC was performed on a Superdex 75 26/600 column (GE Healthcare), equilibrated in PBS.

The untagged variants of VHHs A1 and A2 containing amino acid mutations (SEQ ID NO: 70, 71, 72, 73, 74, 75, 76, 77 and 78) were produced in E. coli WK6 cells transformed with the VHH recombinant expression plasmid and purified from the periplasm by protein A-affinity chromatography and SEC, according to standard protocols such as those in Henry et al. (2016) PLoS One 11(9);e0163113. In short, bacteria were grown in Terrific Broth conditioned medium and VHH expression was induced in the exponential growth phase. During overnight growth VHHs were translocated to the periplasm, from which they were collected with an osmotic shock. VHHs were purified from the periplasmic extract via protein A-affinity chromatography by batch incubation with Amsphere A3 resin (JSR Life Sciences). Following a stepwise acidic elution with 0.1 M glycine-HCl, pH 2.7 the collected eluate fractions were neutralized using 1 M Tris-HCl, pH 8.0, and further purified by SEC on a HiLoad 16/600 Superdex 75 PG column (GE Healthcare) or Superdex 75 10/300 GL (GE Healthcare), equilibrated in PBS.

TABLE 7

Correspondence between VHH variants and SEQ ID

| Clone name (patent) | Variant | SEQ ID |
|---|---|---|
| A1 | HA-tag | 48 |
| | His6-tag | 53 |
| | untagged | 8 |
| | untagged, D59E | |
| | untagged, N64E | |
| | untagged, N64Q | |
| | untagged, N64S | |
| A2 | HA-tag | 49 |
| | His6-tag | 54 |
| | untagged | 9 |
| | untagged; E90Q | |
| | untagged; K29Q, E90Q | |
| | untagged; K29R, E90Q | |
| | untagged; M28A, E90Q | |
| | untagged; M28T, E90Q | |
| A3 | HA-tag | 50 |
| | His6-tag | 55 |
| | untagged | 24 |
| A4 | HA-tag | 51 |
| | His6-tag | 56 |
| | untagged | 32 |
| A5 | HA-tag | 52 |
| | His6-tag | 57 |
| | untagged | 40 |

### Example 2: Knock-in Mice

Human FOLR1 knock-in (KI) mice were generated by the introduction, via homologous recombination, of a human FOLR1 cDNA (NM_016725.3; SEQ ID NO 58, coding for SEQ ID NO 1) after the start codon in exon 4 of the mouse folr1 gene on chromosome 7 (Gene ID: 14275, NCBI accession number NM_001252552.1) in C57BL/6 mice. This disrupts the functional expression of normal mouse folr1 gene, and instead drives the expression of human FOLR1 under the mouse folr1 promotor.

In the KI mice, the region from ATG start codon in exon 4 to part of intron 4 of mouse folr1 is replaced with a “human FOLR1CDS-polyA” cassette.

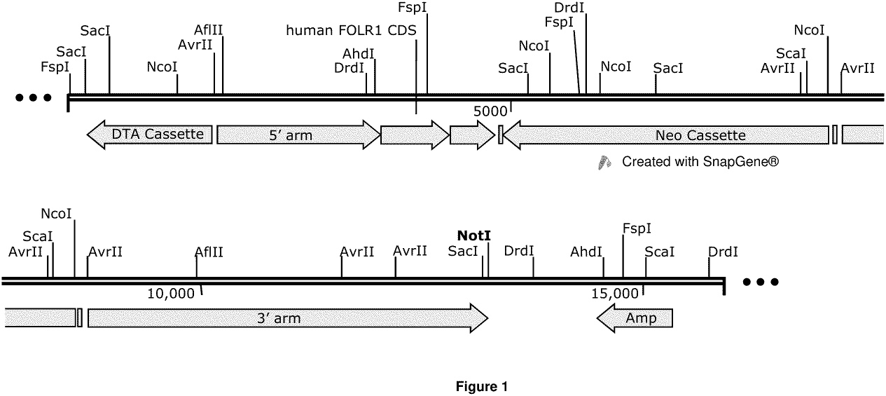

The different elements of the targeting vector are schematically depicted in FIG. 1. The “human FOLR1CDS-polyA” cassette stretches from “5′arm” to “3′arm”. The 15927 bp sequence of the targeting vector is shown in SEQ ID NO 59.

The “human FOLR1CDS-polyA” cassette contains the following relevant elements:
1. The first homology arm (“5′arm” in FIG. 1), encompassing mouse folr1 intron 3 and part of exon 4, is from nucleotides 1704 to 3556. The homology arm is generated by PCR using BAC clone RP23-333J21 or RP23-224L17 from a C57BL/6 BAC library as template.
2. The mouse folr1 exon 4 starts at nucleotide 3549
3. The mouse folr1 start codon is from nucleotide 3557 to 3559
4. The human folr1 coding sequence (derived from NCMI reference sequence NM_016725.3, SEQ ID NO 58), denoted “human FOLR1 CDS” in FIG. 1, is from nucleotides 3557 to 4330
5. The rabbit beta-globin polyadenylation sequence (“rBGpA” in FIG. 1) is from nucleotides 4331 to 4852
6. The Neomycine-resistance gene (“Neo Cassette” in FIG. 1), used for positive selection of ES cells, is between the rBGpA signal sequence and the second homology arm and is flanked by two LoxP sites (“loxP(r)” in FIG. 1, nucleotides 4872-4905 and 8638-8671)
7. The second homology arm (“3′arm” in FIG. 1), encompassing part of mouse folr1 intron 4, exon 5, intron 5, exon 6 and part of intron 6, is from nucleotides 8744 to 13267. The homology arm is generated by PCR using BAC clone RP23-333J21 or RP23-224L17 from a C57BL/6 BAC library as template.

The targeting vector additionally contains:
1. The diphtheria toxin A gene (“DTA Cassette” in FIG. 1), used for negative selection of ES cells not undergoing homologous recombination, located 5′ of the first homology arm
2. An ampicillin-resistance gene (“Amp” in FIG. 1), used for positive selection of the targeting vector in E. coli

The targeting vector is validated by RFLP analysis and sequencing. The targeting vector is linearized with a Notl DNA restriction enzymes and electroporated in C57BL/6N embryonic stem (ES) cells. Individual clones were selected after positive selection on neomycine. The genotype of selected ES clones is validated by karyotype analysis, long-range genomic PCR and southern blot analysis.

Selected ES cell clones were micro-injected into C57BL/6 albino blastocysts, which were then re-implanted into CD-1 pseudo-pregnant females. Male F0 founder mice were identified by their coat color and genomic PCR. F0 founder mice were mated with C57BL/6 females. Upon germline transmission, the neomycine cassette self-deletes from the genome. F1 offspring was genotyped by genomic PCR analysis. F1 heterozygous KI mice were generated from 3 different ES clones and were cross-bred. Heterozygous KI mice bred in the expected mendelian genetic ratios of offspring, indicating the sufficiency and lack of toxicity of the human FOLR1 knock-in construct. Both heterozygous and homozygous genotypes were viable for at least 1 year without any macroscopic abnormalities.

Homozygous deletion of mouse FOLR1 produces embryos with severe growth retardation and multiple developmental abnormalities leading to embryonic lethality (Spiegelstein, O. et al. (2004), Dev. Dyn. 231, 221-231; Tang, L.S. et al. (2005), Am. J. Med. Genet. C Semin. Med. Genet. 135C, 48-58; Zhu, H. et al. (2007), BMC Dev. Biol. 7, 128). In contrast, the in this example described homozygous human FOLR1 knock-in/mouse FOLR1 knockout are viable, with no obvious abnormalities. This indicates that human FOLR1 has functionally compensated for a lack of functional mouse FOLR1.

In wild-type mice, mouse FOLR1 is described to be predominantly localized in epithelial cells of the brain choroid plexus, proximal kidney tubules, ovaries, placenta, and retina (Alam, C et al., Trends Pharmacol Sci. 2020 May;41(5):349-361). It is expected that in the human FOLR1 knock-in mice of this example human FOLR1 is expressed at these sites as well. This is validated by biodistribution studies with radiolabeled anti-human FOLR1 VHHs. Indeed, as explained in example 5, in homozygous and heterozygous human FOLR1 knock-in mice, radioactive anti-human FOLR1 VHH uptake was observed in for instance brain and ovaries where this was not observed in wild type C57BL/6 mice.

### Example 3: Synthesis of Labelled Compound

His6-tagged VHHs are radiolabeled with Technetium-99m (99mTc) for diagnostic purposes, because it emits detectable gamma rays with a photon energy of 140 keV. In short, VHHs are labeled with [99mTc(H2O)3(CO)3] at their His6-tail, as described previously in Xavier et al. Methods Mol Biol. 2012;911:485-90. [99mTc(H2O)3(CO)3] was added to 1 mg/ml VHH solution and incubated for 90 min at 37-50° C. After labeling, the 99mTc-VHH solution was purified on a disposable size-exclusion column pre-equilibrated with PBS to remove unbound [99mTc(H2O)3(CO)3] and passed through a 0.22 µm filter prior to further use. Quality control (QC) was performed by instant thin layer chromatography.

In the examples described below, following human FOLR1-targeting VHHs have been radiolabeled with the diagnostic radioisotope 99mTc, and subsequently characterized in vitro and in vivo for diagnostic applications: His6-tagged VHHs A1, A2, A3, A4 and A5 (SEQ ID NO: 53, 54, 55, 56 and 57 resp.).

VHHs are radiolabeled with 1311 for theranostic purposes, which means that the same radiolabeled VHH can be used for both diagnostic and therapeutic purposes. This can be achieved by applying radioisotopes that emit different types of radiation, that can be used for diagnostic (for example gamma radiation) and therapeutic (for example beta-radiation) purposes. One such a radioisotope is lodine-131 (1311), which emits both gamma-rays of about 364 keV and beta-minus particles with a maximum energy of 606 keV. In short, [131I]SGMIB was synthesized and purified following a procedure adapted from D′Huyvetter M et al. Cancer Res. 2017 Nov 1;23(21):6616-6628. Sodium [131I] iodide was reacted with its trimethylstannyl precursor in acetonitrile for 20 min at RT, after which bisBoc-[131I]SGMIB was deprotected by the addition of trifluoroacetic acid and subsequently purified using reversed-phase HPLC. Purified [131I]SGMIB was incubated with 150 µg VHH in 0.1 M borate buffer pH 8.5 for 20 min at RT, conjugating it to lysine side-chain amine reactive groups via nucleophilic substitution, after which [131I]SGMIB-VHH was purified using a disposable size exclusion column pre-equilibrated with PBS to remove unreacted [131I]SGMIB, and passed through a 0.22 µm filter prior to further use. Quality control (QC) was performed by instant thin layer chromatography.

In the examples described below, following human FOLR1-targeting VHHs have been radiolabeled with the theranostic radioisotope 1311, and subsequently characterized in vitro and in vivo for theranostic applications: (i) His6-tagged VHHs A1, A2, A3, A4 and A5 (SEQ ID NO: 53, 54, 55, 56 and 57 resp.); (ii) untagged VHHs A1, A2 and A3 (SEQ ID NO: 8, 9 and 24 resp.); (iii) untagged variants from VHHs A1 and A2 containing amino acids mutations: A1-D59E, A1-N64E, A1-N64Q, A1-N64S, A2-E90Q, A2-K29Q-E90Q, A2-K29R-E90Q, A2-M28A-E90Q and A2-M28T-E90Q (SEQ ID NO: 70, 71, 72, 73, 74, 75, 76, 77 and 78 resp.).

VHHs are also radiolabeled with 177Lu for theranostic purposes (D′Huyvetter et al. (2012) Contrast Media Mol Imaging 7(2):254-264). Lutetium-177 emits both gamma-rays of about 113 and 210 keV and beta-minus particles with a maximum energy of 497 keV. In short, the bifunctional chelator p-SCN-Bn-DOTA was conjugated to lysine side-chain amine reactive groups of the VHH in a 0.05 M sodium carbonate buffer (pH 8.5). After size exclusion purification, resulting VHH-DOTA was reconstituted in 0.1 M ammonium acetate buffer pH 7.0. The necessary amount of 177Lu was added to a test vial containing metal-free 0.1 M ammonium acetate buffer pH 5.0. Then, 25-100 µg of VHH-DOTA was added and incubated for 30 min at 55° C. 177Lu-DOTA-VHH was purified via size exclusion purification and passed through a 0.22 µm filter prior to further use. Quality control (QC) was performed by instant thin layer chromatography.

In example 7a described below, human FOLR1-targeting VHHs A1 and A2 (SEQ ID NO: 8 and 9) have been radiolabeled with theranostic radioisotope 177Lu, and subsequently characterized in vivo for theranostic applications.

Finally, VHHs are also radiolabeled with 225Ac for therapeutic purposes (Pruszynski et al. (2018) Mol Pharm 15(4):1457-1466). Actinium-225 emits alpha particles of 5.8 MeV. In short, the bifunctional chelator p-SCN-Bn-DOTA was conjugated to lysine side-chain amine reactive groups of the VHH in a 0.05 M sodium carbonate buffer (pH 8.5). After size exclusion purification, resulting VHH-DOTA was reconstituted in 0.1 M ammonium acetate buffer pH 7.0. The desired activity of 225Ac was added to a test vial containing 0.8 M ammonium acetate (pH 5.0) followed by the incubation with VHH-DOTA (25 - 100 µg) for 90 min at 55° C. The mixture was cooled to RT and quenched with 50 mM DTPA (in 0.8 M ammonium acetate) and Chelex 100 in order to complex any free 225Ac. 225Ac-DOTA-VHH was purified via size exclusion purification and passed through a 0.22 µm filter prior to further use. Quality control (QC) was performed by instant thin layer chromatography.

In example 7b described below, human FOLR1-targeting VHH A1 (SEQ ID NO: 8) has been radiolabeled with the therapeutic radioisotope 225Ac, and subsequently characterized in vivo for therapeutic applications:

### Example 4: Binding Activities of the VHH or of the Labelled Compound Comprising the VHH

### Example 4a

This part of the example describes the specific binding of VHHs A1, A2, A3, A4 and A5 to the extracellular domain of human FOLR1 (NCBI reference sequence NP_057937.1) and absence of binding to the extracellular domain of human FOLR2 (NCBI reference sequence NP_000794), human FOLR3 (NCBI reference sequence NP_000795) and murine FOLR1 (NCBI reference sequence NP_032060.2), as tested in ELISA.

The day before, 0.1 µg recombinant protein at a concentration of 1 µg/mL in 100 mM NaHCO3, pH 8.2 was coated in a 96-well ELISA plate (Nunc MaxiSorp). Per VHH clone also a blank coated well was foreseen (only buffer). The wells were overcoated with protein-free T20 (PBS) blocking buffer (Pierce). After washing with PBS, pH7.4, 0.05% Tween, VHH-containing bacterial extract was added to every well. Binding of HA-tagged VHHs was detected by using mouse anti-HA.11 epitope tag (clone 16B12, Biolegend) as the primary Ab and goat anti-mouse IgG (whole molecule) alkaline phosphatase conjugate (Sigma-Aldrich) as the secondary Ab, with thorough washing with PBS, pH 7.4, 0.05% Tween in between. Signals were developed using phosphatase substrate (Sigma-Aldrich) in AP blot buffer (100 mM NaCl, 50 mM MgCl2, 100 mM Tris, pH 9.5). The absorbance was determined at 405 nm using an absorbance microplate reader (Molecular Devices). Per clone the ratio was determined between the absorbance in the antigen-coated well versus the well without antigen.

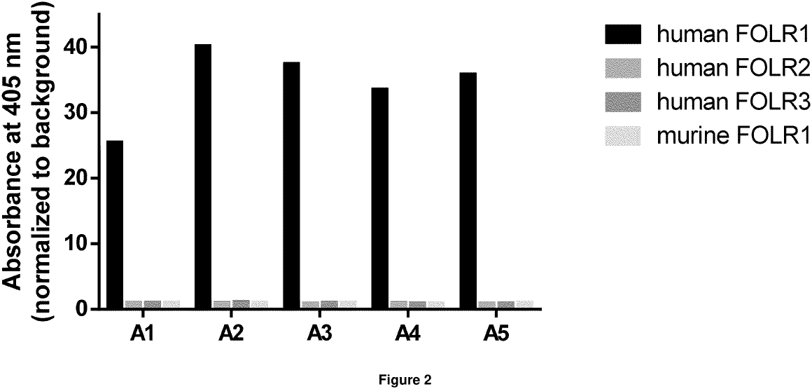

As show in FIG. 2, VHHs A1, A2, A3, A4 and A5 (SEQ ID NO: 48, 49, 50, 51 and 52 resp.) showed signal-to-background ratios greater than 20 for wells coated with human FOLR1 recombinant protein (Sino Biological), confirming specific binding to human FOLR1. All VHHs showed absence of specific binding to human FOLR2 (Sino Biological), human FOLR3 (R&D Systems) and murine FOLR1 (Sino Biological), with signal-to-background ratios around 1.

### Example 4b

This part of the example describes the in vitro cell binding in presence or absence of folic acid, the natural ligand of human FOLR1.

The ability of the VHHs to target the naturally expressed receptor was tested on the human FOLR1-expressing ovarian carcinoma cell line SKOV-3 in flow cytometry experiments. For testing in flow cytometry, purified His₆-tagged VHHs A1, A2, A3, A4, A5 (SEQ ID NO: 53, 54, 55, 56 and 57 resp.) were prepared at 1 µM in PBS, 0.5% BSA. The VHH samples (100 µl final volume) were pre-incubated with the primary mouse anti-Histidine tag antibody (clone AD1.1.10, Bio-Rad). Per test condition 10⁵ to 2.10⁵ cells, washed in PBS, 0.5% BSA, were pelleted in round-bottom tubes. Next, the cell pellets were resuspended and incubated with the VHH-primary antibody mixture. Binding of the VHHs was detected by using PE-conjugated rat anti-mouse IgG1 (clone A85-1, BD Pharmingen) as secondary detection antibody, with thorough washing in between and finally dissolved in PBS, 0.5% BSA. After addition of 7-AAD (BD Pharmingen), flow cytometry was performed on a FACS Canto II (BD Biosciences). Data were analyzed using FlowJo software. Based on the forward scatter - side scatter plot a single cell gate was drawn, followed by selection of live cells by gating for PerCP-Cy5.5 negative population. The median fluorescence intensity was determined on a histogram for the phycoerythrin signal of the live cells. A compensation matrix was applied to correct for PerCP-Cy5.5 signal in the phycoerythrin window. The difference in median fluorescence intensity (Δmfi) was calculated relative to a test condition without incubation of VHHs (cells + primary Ab + secondary Ab), and are shown in FIG. 3.

In case of a condition in which folic acid competition was tested, the cells were first pre-incubated in PBS, 0.5% BSA supplemented with 100 µM folic acid, and the prepared VHH samples also contained 100 µM folic acid (Acros Organics).

All VHHs A1, A2, A3, A4 and A5 were able to target the human FOLR1-expressing cell line SKOV-3. The presence of a 100-fold excess of folic acid did not interfere with VHH binding. This means that the VHH could bind to human FOLR1-expressing cells in vivo while circulating folic acid levels in the serum are high.

### Example 4c

This part of the example describes the determination of competitive binding characteristics of His₆-tagged VHHs.

Competitive binding characteristics of His₆-tagged VHHs were determined via surface plasmon resonance with a Biacore T200 instrument (GE Healthcare). Immobilization of human FOLR1 recombinant protein (Sino Biological) and regeneration were performed as described below for kinetic measurements.

Measurements were performed in dual injection mode. In a first step the ligand is saturated by injection of a 250 nM solution of analyte 1. In the second step an equimolar mixture of analyte 1 and 2 at 250 nM is injected. Contact times were 180 s for every step.

Side-by-side comparison of VHHs A1, A2, A3, A4 and A5 (SEQ ID NO: 53, 54, 55, 56 and 57 resp.) in table 8 shows loss of binding to human FOLR1, after the receptor was bound by any of the other VHHs. These data illustrate the VHHs A1, A2, A3, A4 and A5 bind an overlapping epitope.

Table 8: Side-by-side comparison of His₆-tagged VHHs A1, A2, A3, A4, A5 for binding to human FOLR1 to identify competitive binding characteristics. Binding was measured in surface plasmon resonance with immobilized human FOLR1 recombinant protein as the ligand. After saturation of the ligand with VHH 1 an equimolar mixture of VHH 1 and VHH 2 was injected. Percentages express the loss in response level of VHH 2, relative to the response when VHH 2 was injected alone.

| | | 2nd VHH | | | | |
|---|---|---|---|---|---|---|
| | | A1 | A2 | A3 | A4 | A5 |
| 1st VHH | A1 | | -96% | -97% | -97% | -96% |
| | A2 | -98% | | n.d. | -99% | -99% |
| | A3 | -100% | -99% | | -100% | -100% |
| | A4 | -98% | -96% | -97% | | -98% |
| | A5 | -91% | -86% | -89% | -91% | |

### Example 4d

This part of the example describes the determination of antigen binding kinetics, and its effect on human FOLR1 targeting behavior in vivo.

The kinetic parameters of antigen binding by purified VHHs were determined via surface plasmon resonance with a Biacore T200 instrument (GE Healthcare). The human FOLR1 recombinant protein (Sino Biological) was immobilized in 10 mM sodium acetate, pH 5.5 on a CM5 chip (GE Healthcare) via amine coupling chemistry to 500 RU. His₆-tagged VHHs were analyzed in a twofold serial dilution in HBS (125 nM-0.488 nM for VHHs A4 and A5; 10 nM-0.039 nM for VHHs A1, A2 and A3) with an analyte flow rate of 30 µl/min. The association phase took 180 s (A4-A5) or 450 s (A1-A3) and the dissociation phase 600 s (A4-A5) or 900 s (A1-A3). Regeneration was done by a single injection of 50 mM glycine-HCl, pH 2.5, 2 M MgCl₂ for 30 s at 30 µl/min, followed by 120 s stabilization. Binding curves were fitted using a ‘1:1 (antigen:analyte)’ binding model in Biacore T200 evaluation software.

Table 9 gives an overview of the kinetic parameters of antigen binding, as determined by surface plasmon resonance with immobilized human FOLR1 recombinant protein. His₆-tagged VHHs A1, A2, A3 and A4 (SEQ ID NO: 53, 54, 55 and 56 resp.) showed at least nanomolar affinities with equilibrium dissociation constants (K_{D}) that were smaller than 5 nM. In particular best binding characteristics were observed for VHHs A1 and A2 with K_{D} values of 40 pM and 240 pM, respectively, and dissociation reaction rate constants (k_{d}) < 5 10⁻⁴ s⁻¹. VHH A5 (SEQ ID NO: 57) had an affinity (K_{D}) of 14 nM and k_{d} that was ≥ 10⁻² s⁻¹.

TABLE 9

Kinetic binding parameters ka, kd and KD of purified, His₆-tagged VHHs A1, A2, A3, A4 and A5

| VHH | kₐ (M⁻¹ s⁻¹) | k_{d} (s⁻¹) | K_{D} (M) | Rmax (RU) | Chi² (RU²) |
|---|---|---|---|---|---|
| A1 | 3.6 E+06 | 1.4 E-04 | 4.0 E-11 | 164.7 | 1.71 |
| A2 | 1.3 E+06 | 3.3 E-04 | 2.4 E-10 | 168.1 | 1.99 |
| A3 | 1.2 E+06 | 1.1 E-03 | 9.2 E-10 | 157.1 | 0.31 |
| A4 | 1.3 E+06 | 5.7 E-03 | 4.3 E-9 | 143.7 | 0.31 |
| A5 | 1.3 E+06 | 1.8 E-02 | 1.4 E-8 | 135.9 | 0.99 |

Next, we describe the in vivo human FOLR1 tumor targeting after radiolabelling with the diagnostic radioisotope ^{99m}Tc. The in vivo tumor targeting of human FOLR1-targeting VHHs was assessed via necropsy studies in athymic nude mice bearing FOLR1^{pos} SKOV-3 tumors.

His₆-tagged human FOLR1-targeting VHHs A1, A2, A3, A4 and A5 (SEQ ID NO: 53, 54, 55, 56 and 57 resp.) were radiolabelled with the diagnostic radioisotope ^{99m}Tc, according to the radiochemical procedure described in example 3.

After quality control (> 95%), human FOLR1^{pos} SKOV-3 cancer cells were incubated with serial dilutions with concentrations ranging from 0 to 300 nM of the different ^{99m}Tc-labelled VHHs. A 100-fold excess of the corresponding unlabeled VHH was added in parallel to saturate the human FOLR1 receptors expressed on cancer cells, to assess non-specific binding.

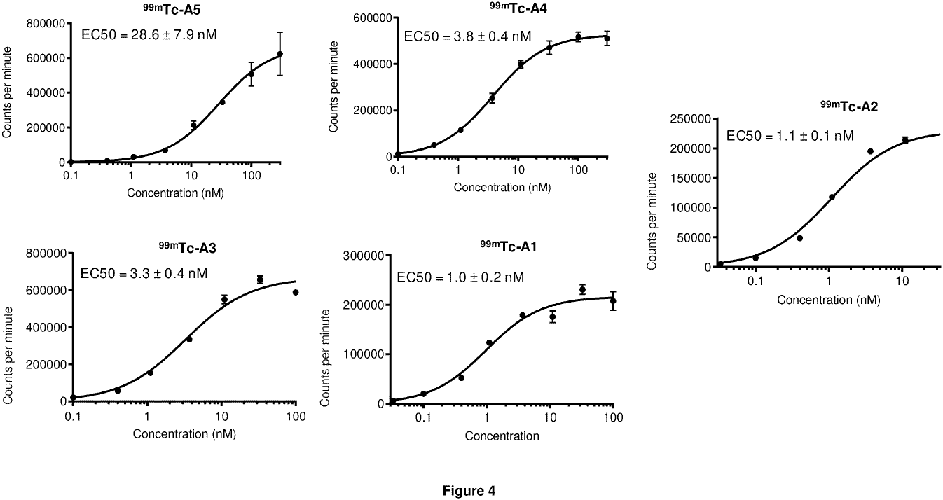

EC50 of the VHHs on human FOLR1^{pos} SKOV-3 cancer cells calculated 28.6 ± 7.9 nM (A5), 3.8 ± 0.4 nM (A4), 3.3 ± 0.4 nM (A3), 1.0 ± 0.2 (A1) and 1.1 ± 0.1 nM (A2). indicating that the EC50 of both A1 and A2 calculated about 1 nM, and those of both A4 and A3 slightly more inferior, ranging 3 nM, as shown in FIG. 4.

The different ^{99m}Tc-labeled human FOLR1-targeting VHHs were evaluated in vivo in athymic nude mice bearing human FOLR1^{pos} SKOV-3 tumors via SPECT/CT imaging and dissection studies.

To this, athymic nude mice (n=3 per time point/per VHH) were inoculated subcutaneously with human FOLR1^{pos} SKOV-3 tumor cells in the right hind leg. After validation of tumor growth (about 100 mm³), all mice were intravenously injected in the tail vein with about 1 mCi (± 5 µg) of the different ^{99m}Tc-labeled VHHs. Next, SPECT/CT was performed 1 h after tracer administration using a Vector⁺/CT MILabs system. SPECT/CT imaging was performed using a SPECT collimator and a spiral scan mode of six bed positions (2 min30 s per position). For CT, a normal scan mode of only one position was used. The obtained SPECT data are reconstructed with a 0.4 voxel size, 2 subsets and 4 iterations, after which images are fused and corrected for attenuation based on the CT scan. Images are analyzed using a medical image data analysis tool (AMIDE). Uptake values of the anti-human FOLR1 VHH radioconjugates in a selection of organs and tissues were analyzed and expressed as % injected activity per cm³ (%IA/cc).

Next, mice were euthanized by cervical dislocation after 1.5 h, dissected after which different organs and tissues were collected. Organs and tissues of interest were weighed and measured for radioactivity using an automatic gamma counter, along with injection standards. Results were expressed as % of injected activity (IA)/g tissue.

All ^{99m}Tc-labeled, His₆-tagged, human FOLR1-targeting VHHs A1-A5 revealed a biodistribution in mice that is typical for a radiolabeled VHH (Tables 10 and 11). Low radioactive signal was measured in all organs and tissues except for kidneys and tumor. All human FOLR1-targeting VHHs accumulate specifically in tumors, with uptake values significantly higher (unpaired t-test, p < 0.05) compared to the value obtained for nontargeting control VHH R3B23 (Lemaire et al. (2014) Leukemia 28(2):444-7). ^{99m}Tc-labeled A3 and A1 calculated uptake values in tumor (2.94 ± 0.92 and 3.85 ± 1.75%IA/g) that were higher, but however not significantly higher, compared to that of ^{99m}Tc-A4 (1.52 ± 0.52%IA/g) (p=0.0677, one-way ANOVA). In the case of ^{99m}Tc-A2, tumor uptake was significantly higher compared to that obtained for ^{99m}Tc-A4 (p < 0.05).

TABLE 10

Uptake in different organs and tissues obtained from SPECT/CT images 1 h after injection of ^{99m}Tc-labeled His₆-tagged VHHs in athymic nude mice bearing human FOLR1^{pos} SKOV-3 tumors

| %IA/cc | ^{99m}Tc-A5 | | ^{99m}Tc-A4 | | ^{99m}Tc-A3 | | ^{99m}Tc-A1 | | ^{99m}Tc-A2 | | ^{99m}Tc- R3B23 | |
|---|---|---|---|---|---|---|---|---|---|---|---|---|
| Organ/tissue | Mean | SD | Mean | SD | Mean | SD | Mean | SD | Mean | SD | Mean | SD |
| Kidney | 31.11 | 2.67 | 43.09 | 9.26 | 42.01 | 0.85 | 69.63 | 3.04 | 46.82 | 3.11 | 55.36 | 1.32 |
| Heart | 0.30 | 0.07 | 0.43 | 0.02 | 0.30 | 0.03 | 0.24 | 0.05 | 0.31 | 0.07 | 0.21 | 0.02 |
| Liver | 0.42 | 0.01 | 0.66 | 0.07 | 1.26 | 0.07 | 1.24 | 0.31 | 0.66 | 0.17 | 0.17 | 0.04 |
| Lung | 0.26 | 0.07 | 0.33 | 0.01 | 0.28 | 0.07 | 0.28 | 0.06 | 0.24 | 0.05 | 0.19 | 0.04 |
| Muscle | 0.10 | 0.02 | 0.20 | 0.03 | 0.11 | 0.02 | 0.17 | 0.04 | 0.15 | 0.08 | 0.07 | 0.02 |
| Tumor | 0.50 | 0.19 | 1.03 | 0.27 | 1.39 | 0.47 | 1.61 | 0.56 | 1.56 | 0.29 | 0.15 | 0.003 |

Data expressed as %IA/cc and presented as mean ± SD (n = 3).

TABLE 11

Uptake in different organs and tissues obtained from dissections 1.5 h after injection of ^{99m}Tc-labeled His₆-tagged VHHs in athymic nude mice bearing human FOLR1^{pos} SKOV-3 tumors

| %IA/g | ^{99m}Tc-A5 | | ^{99m}Tc-A4 | | ^{99m}Tc-A3 | | ^{99m}Tc-A1 | | ^{99m}Tc-A2 | | ^{99m}Tc- R3B23 | |
|---|---|---|---|---|---|---|---|---|---|---|---|---|
| Organ/tissue | Mean | SD | Mean | SD | Mean | SD | Mean | SD | Mean | SD | Mean | SD |
| Blood | 0.38 | 0.06 | 0.39 | 0.08 | 0.49 | 0.06 | 0.26 | 0.06 | 0.42 | 0.10 | 0.26 | 0.02 |
| Lymphnodes | 0.20 | 0.10 | 0.56 | 0.41 | 0.68 | 0.67 | 0.55 | 0.48 | 0.51 | 0.21 | 0.27 | 0.02 |
| Heart | 0.18 | 0.03 | 0.21 | 0.03 | 0.26 | 0.02 | 0.25 | 0.04 | 0.24 | 0.08 | 0.13 | 0.01 |
| Lung | 0.35 | 0.04 | 0.49 | 0.02 | 0.62 | 0.15 | 0.80 | 0.16 | 0.57 | 0.13 | 0.35 | 0.02 |
| Galbladder | 0.50 | 0.18 | 1.45 | 0.83 | 1.63 | 1.05 | 1.26 | 0.45 | 1.86 | 0.74 | 0.30 | 0.05 |
| Liver | 0.62 | 0.02 | 0.77 | 0.10 | 2.75 | 0.23 | 2.49 | 0.48 | 1.29 | 0.16 | 0.28 | 0.02 |
| Pancreas | 0.15 | 0.07 | 0.20 | 0.09 | 0.11 | 0.01 | 0.17 | 0.03 | 0.15 | 0.04 | 0.07 | 0.003 |
| Spleen | 0.17 | 0.03 | 0.24 | 0.004 | 0.53 | 0.03 | 0.41 | 0.06 | 0.27 | 0.07 | 0.10 | 0.01 |
| Adrenals | 0.31 | 0.07 | 2.23 | 3.22 | 0.72 | 0.05 | 0.59 | 0.03 | 0.46 | 0.14 | 0.14 | 0.01 |
| Kidney | 88.09 | 10.00 | 107.62 | 38.74 | 190.66 | 10.12 | 252.02 | 34.10 | 231.77 | 26.58 | 169.32 | 10.25 |
| Stomach | 0.53 | 0.19 | 0.41 | 0.03 | 0.28 | 0.01 | 0.41 | 0.07 | 0.59 | 0.08 | 0.32 | 0.06 |
| Small intestine | 0.32 | 0.19 | 0.34 | 0.06 | 0.27 | 0.04 | 0.40 | 0.05 | 0.32 | 0.09 | 0.12 | 0.02 |
| Large intestine | 0.22 | 0.03 | 0.30 | 0.03 | 0.27 | 0.02 | 0.47 | 0.09 | 0.32 | 0.06 | 0.15 | 0.02 |
| Uterus | 0.67 | 0.25 | 0.61 | 0.23 | 1.75 | 0.99 | 0.89 | 0.16 | 1.40 | 0.61 | 0.53 | 0.03 |
| Skin | 0.39 | 0.07 | 0.38 | 0.05 | 0.67 | 0.48 | 0.49 | 0.07 | 0.53 | 0.13 | 0.26 | 0.06 |
| Muscle | 0.12 | 0.02 | 0.20 | 0.13 | 0.18 | 0.04 | 0.19 | 0.05 | 0.39 | 0.15 | 0.09 | 0.03 |
| Bone | 0.13 | 0.08 | 0.22 | 0.11 | 0.29 | 0.07 | 0.21 | 0.01 | 0.26 | 0.05 | 0.08 | 0.01 |
| Tumor | 0.55 | 0.11 | 1.52 | 0.57 | 2.94 | 0.92 | 3.85 | 1.75 | 4.11 | 0.62 | 0.19 | 0.01 |
| Brain | 0.02 | 0.004 | 0.02 | 0.01 | 0.22 | 0.34 | 0.02 | 0.003 | 0.03 | 0.01 | 0.01 | 0.002 |

Data expressed as %IA/g and presented as mean ± SD (n = 3).

Taken together, this example demonstrates that VHHs require at least nanomolar affinity (K_{D} ≤ 5 nM) and antigen binding kinetics with a dissociation reaction rate constant k_{d} < 10⁻² s⁻¹ in order to selectively target human FOLR1, with suitable tumor accumulation when for example expressed on tumor tissue.

### Example 4e:

The kinetic parameters of antigen binding for purified VHHs A1, A2 and A3 were determined via surface plasmon resonance with a Biacore T200 instrument (GE Healthcare) using single cycle kinetics mode. While example 4d provides a first and preferred way of assessing the kinetic parameters of antigen binding for VHHs using surface plasmon resonance, this example provides an alternative setup for using surface plasmon resonance to assess the kinetic parameters of antigen binding, inverting the analyte and immobilized ligand. His₆-tagged VHH A1, A2 or A3 was immobilized in 10 mM sodium acetate, pH 5.5 on a CM5 chip (GE Healthcare) via amine coupling chemistry to 160 RU. The human FOLR1 recombinant protein (Sino Biological) was analyzed in a threefold serial dilution in HBS (38.5 nM-0.47 nM) with an analyte flow rate of 30 µl/min. The association phase took 180 s for every concentration and the dissociation phase 900 s. Regeneration was done by a single injection of 50 mM glycine-HCl, pH 2.5, 2 M MgCl2 for 30 s at 30 µl/min, followed by 120 s stabilization. Binding curves were fitted using a ‘1:1 (antigen:analyte)’ binding model in Biacore T200 evaluation software.

Table 12 gives an overview of the kinetic parameters of antigen binding, as determined by surface plasmon resonance with immobilized His₆-tagged VHH A1 (SEQ ID NO: 53), VHH A2 (SEQ ID NO: 54) or VHH A3 (SEQ ID NO: 55). The VHHs showed at least nanomolar affinities for human FOLR1 recombinant protein, with dissociation reaction rate constants (k_{d}) < 10⁻³ s⁻¹.

TABLE 12

Kinetic binding parameters kₐ, k_{d} and K_{D} of purified, His₆-tagged VHH A1, A2 and A3

| VHH | kₐ (M⁻¹ s⁻¹) | k_{d} (s⁻¹) | K_{D} (M) | Rₘₐₓ (RU) | Chi² (RU²) |
|---|---|---|---|---|---|
| A1 | 4.3 E+05 | 1.9 E-04 | 4.4 E-10 | 254.7 | 4.06 |
| A2 | 4.4 E+05 | 6.8 E-04 | 1.5 E-9 | 282.3 | 5.07 |
| A3 | 3.3 E+05 | 6.3 E-04 | 1.9 E-9 | 252.8 | 1.38 |

The value of the equilibrium dissociation constant (K_{D}) for antigen-binding VHH A1 using the method of example 4d is 4.0 E-11 M wherease the one determined in example 4e is 4.4 E-10 M. This factor 10 difference in affinity is mainly attributable to the difference in the value of the association reaction rate constant (kₐ) and is probably due to the experimental setup of the surface plasmon resonance assay (immobilisation of the antigen in example 4d versus immobilisation of the antigen-binding VHH A1 in example 4e), impeding antigen binding in the setup of example 4e.

However, the value of the dissociation reaction rate constant (k_{d}) of antigen-binding VHH A1 using the experimental setup of example 4d is 1.4 E-4 s⁻¹, wherease the one determined in example 4e is 1.9 E-4 s⁻¹, so very similar and thus not effected by the experimental setup.

The kinetic parameters of antigen binding of full length antibodies against human FOLR1 known in the prior art have already been assessed in US 2014/0205610 using surface plasmon resonance in a similar setup as in example 4e, by immobilizing the antibody. Ab ChRA15-7Acc (US 2014/0205610, example 29) and Ab HuRA15-7CTAcc (US 2014/0205610, example 49) exhibit the same affinity after sequence modification/optimization.

The full length antibodies of US 2014/0205610 and the VHH A1 of the invention have different dissociation reaction rate constants (k_{d}): 1.08 E-03 s⁻¹ in table 3 of US 2014/0205610 versus 1.9 E-04 s⁻¹ in table 12 of example 4e above, respectively. It means that there is a factor 10 difference in favour of the antigen binding VHH A1, that dissociates 10x slower from its antigen than the full length antibody of US 2014/0205610.

### Example 5: Diagnostic Use of the Labelled Compound

In this example we describe the diagnostic potential of several human FOLR1-targeting VHHs by investigating their ability to visualise human FOLR1-expression after radiolabelling with the diagnostic radioisotope ^{99m}Tc and in a second part with the theranostic radionuclide ¹³¹I. The diagnostic potential is confirmed both in relevant human FOLR1 knock-in mice from example 2 as well as in athymic nude mice with human FOLR1-expressing SKOV-3 tumors.

Part one of the example describes the evaluation of ^{99m}Tc-labeled, His₆-tagged VHH A1 in wildtype, hetero-zygote and homozygous human FOLR1 knock-in mice. In a second part, the biodistribution and human FOLR1-targeting of ^{99m}Tc-labeled, His₆-tagged VHHs A1 and A2 was compared to that of ^{99m}Tc-labeled non-targeting control VHH R3B23 in homozygous human FOLR1 knock-in mice. Finally, the capacity of both ¹³¹I-labeled untagged VHHs A1 and A2 to accumulate in human FOLR1-expressing tumors was evaluated in athymic nude mice bearing human FOLR1^{pos} SKOV-3 tumors

In a first part of this example, the diagnostic potential of radiolabeled VHH A1 was evaluated in three distinct mouse breeds with variable expression of human FOLR1, as described in example 2.

To this, his₆-tagged FOLR1-targeting VHH A1 (SEQ ID NO 53) was radiolabelled with the diagnostic radioisotope ^{99m}Tc, according to the radiochemical procedure described in example 3. After quality control (> 95%), a group of wild-type, heterozygous and homozygous human FOLR1 knock-in mice (from example 2) were intravenously injected in the tail vein with about 1.3 mCi (± 5 µg) of ^{99m}Tc-labelled VHH A1. Next, SPECT/CT was performed 1 h after tracer administration using a Vector⁺/CT MILabs system. SPECT/CT imaging was performed using a SPECT collimator and a spiral scan mode of six bed positions (2min30s per position). For CT, a normal scan mode of only one position was used. The obtained SPECT data are reconstructed with a 0.4 voxel size, 2 subsets and 4 iterations, after which images are fused and corrected for attenuation based on the CT scan. Images are analyzed using a medical image data analysis tool (AMIDE). Uptake values in a selection of organs and tissues were analyzed and expressed as % injected activity per cm³ (%IA/cc).

Next, mice were euthanized by cervical dislocation after 1.5 h, dissected after which different organs and tissues were collected. Organs and tissues of interest were weighed and measured for radioactivity using an automatic gamma counter, along with injection standards. Results were expressed as % of injected activity (IA)/g tissue.

Image quantification (table 13) and results obtained via dissections (table 14) indicated that the administration of ^{99m}Tc-VHH A1 reveals focalised uptake in brain and an elevated uptake in ovaries in both homozygote and heterozygote human FOLR1 knock-in mice, while this elevated uptake is absent in the wildtype mice. More specifically, based on the dissection studies, the uptake in brain of homozygous human FOLR1 KI-mice is significant higher compared to uptake in wildtypes (p=0.014, one-way ANOVA, multiple comparisons), while uptake in heterozygote human FOLR1 KI-mice is not significantly higher compared to wildtypes (p=0.056, one-way ANOVA, multiple comparisons). These observations are confirmed with data obtained from corresponding image quantification, with the uptake in brain of homozygous KI-mice significantly higher compared to wild-types (p=0.007), while the uptake in brain was not significantly higher in heterozygous human FOLR1 KI-mice versus wild-type mice (p=0.3120).

TABLE 13

Uptake in different organs and tissues obtained from SPECT/CT images 1 h after i.v. injection of ^{99m}Tc-VHH A1 in wildtype mice and homo- and heterozygous human FOLR1 knock-in mice

| %IA/cc | ^{99m}Tc-VHH A1 in homozygote human FOLR1 KI mice | | ^{99m}Tc-VHH A1 in heterozygote human FOLR1 KI mice | | ^{99m}Tc-VHH A1 in wild type mice | |
|---|---|---|---|---|---|---|
| | Mean | ± SD | Mean | ± SD | Mean | ± SD |
| Kidney | 122.23 | 9.03 | 99.86 | 11.22 | 87.98 | 5.75 |
| Muscle | 0.20 | 0.04 | 0.16 | 0.02 | 0.11 | 0.02 |
| Heart | 0.59 | 0.16 | 0.33 | 0.07 | 0.39 | 0.10 |
| Lung | 0.46 | 0.16 | 0.37 | 0.11 | 0.35 | 0.06 |
| Liver | 3.31 | 0.24 | 3.15 | 0.47 | 2.78 | 0.48 |
| Brain | 0.89 | 0.36 | 0.36 | 0.07 | 0.11 | 0.03 |

Data expressed as %IA/cc and presented as mean ± SD (n = 3)

TABLE 14

Uptake in different organs and tissues obtained from dissections 1.5 h after i.v. injection of ^{99m}Tc-VHH A1 in wildtype mice and homo- and heterozygous human FOLR1 knock-in mice)

| %IA/g | ^{99m}Tc-VHH A1 in homozygous human FOLR1 KI mice | | ^{99m}Tc-VHH A1 in heterozygous human FOLR1 KI mice | | ^{99m}Tc-VHH A1 wild-type mice | |
|---|---|---|---|---|---|---|
| | Mean | ± SD | Mean | ± SD | Mean | ± SD |
| Blood | 0.50 | 0.04 | 0.55 | 0.04 | 0.65 | 0.02 |
| Lymphnodes | 1.48 | 1.77 | 0.85 | 0.37 | 0.53 | 0.20 |
| Thymus | 0.52 | 0.10 | 0.36 | 0.04 | 0.55 | 0.06 |
| Heart | 0.52 | 0.04 | 0.67 | 0.10 | 0.70 | 0.17 |
| Lung | 1.95 | 0.21 | 1.87 | 0.10 | 2.28 | 0.58 |
| Liver | 8.30 | 0.85 | 7.95 | 1.08 | 9.10 | 1.36 |
| Pancreas | 0.81 | 0.10 | 0.77 | 0.17 | 0.52 | 0.08 |
| Spleen | 1.98 | 0.26 | 1.82 | 0.30 | 1.97 | 0.08 |
| Kidney | 463.03 | 34.22 | 429.83 | 47.38 | 494.45 | 30.32 |
| Stomach | 1.08 | 0.14 | 3.77 | 4.53 | 1.32 | 0.11 |
| Small intestine | 1.75 | 0.60 | 1.39 | 0.22 | 1.35 | 0.11 |
| Large intestine | 1.73 | 0.38 | 2.16 | 0.30 | 1.88 | 0.20 |
| Ovary | 11.80 | 12.31 | 33.12 | 51.90 | 1.12 | 0.89 |
| Uterus | 2.14 | 0.76 | 1.54 | 0.13 | 1.43 | 0.42 |
| Skin | 0.93 | 0.35 | 1.06 | 0.45 | 1.35 | 0.58 |
| Muscle | 0.36 | 0.08 | 0.31 | 0.09 | 0.37 | 0.03 |
| Bone | 0.83 | 0.12 | 0.75 | 0.14 | 1.11 | 0.58 |
| Joint | 1.03 | 0.11 | 1.01 | 0.08 | 1.50 | 0.50 |
| Brain | 0.51 | 0.22 | 0.33 | 0.08 | 0.06 | 0.04 |

Data expressed as %IA/g and presented as mean ± SD (n = 3

In a second part of this example, both human FOLR1-targeting, His₆-tagged VHHs A1 and A2 (SEQ ID NO 53 and 54, resp.), as well as the non-targeting control VHH R3B23 were radiolabelled with ^{99m}Tc, after which their biodistribution was assessed via dissections in homozygous human FOLR1 knock-in mice. The experimental procedure was identical to that described in the first part of this example, except for the incubation temperature during radiolabelling which was decreased to 37° C. in this latter case.

Image quantification (table 15) and results obtained via dissections (table 16) indicated that the administration of ^{99m}Tc-labelled VHHs A1 and A2 led to focalised uptake in brain and an elevated uptake in ovaries in homozygote mice, which was specific for these human FOLR1-targeting VHH, as this elevated uptake was found absent for ^{99m}Tc-labelled non-targeting control VHH R3B23 administered to the same mice. Uptake in brain was significantly higher in these mice for both VHHs A1 and A2 compared to R3B23 (one-way ANOVA, multiple comparisons; dissection data: p < 0.0001; image quantification: p < 0.0001).

TABLE 15

Uptake in different organs and tissues obtained from SPECT/CT images 1.5 h after i.v. injection of ^{99m}Tc-VHHs in homozygote human FOLR1 knock-in mice

| %IA/cc 1 h p.i. | ^{99m}Tc-VHH A1 | | ^{99m}Tc-VHH A2 | | ^{99m}Tc-VHH R3B23 | |
|---|---|---|---|---|---|---|
| | Mean | ± SD | Mean | ± SD | Mean | ± SD |
| Kidney | 107.73 | 7.05 | 101.42 | 1.24 | 67.36 | 5.93 |
| Liver | 1.07 | 0.05 | 0.71 | 0.01 | 0.40 | 0.07 |
| Heart | 0.42 | 0.03 | 0.49 | 0.01 | 0.35 | 0.05 |
| Lung | 0.42 | 0.09 | 0.36 | 0.03 | 0.21 | 0.01 |
| Bone | 0.19 | 0.03 | 0.20 | 0.03 | 0.23 | 0.13 |
| Muscle | 0.17 | 0.08 | 0.12 | 0.03 | 0.13 | 0.02 |
| Brain | 0.63 | 0.07 | 0.59 | 0.03 | 0.07 | 0.01 |

Data expressed as %IA/cc and presented as mean ± SD (n = 3)

TABLE 16

Uptake in different organs and tissues obtained from dissections 1 h after i.v. injection of ^{99m}Tc-VHHs in homozygote human FOLR1 knock-in mice

| %IA/g 1.5 h p.i. | ^{99m}Tc-VHH A1 | | ^{99m}Tc-VHH A2 | | ^{99m}Tc-VHH R3B23 | |
|---|---|---|---|---|---|---|
| | Mean | ± SD | Mean | ± SD | Mean | ± SD |
| Blood | 0.66 | 0.05 | 0.84 | 0.03 | 0.83 | 0.05 |
| Lymphnodes | 1.03 | 0.21 | 0.49 | 0.19 | 0.43 | 0.23 |
| Thymus | 0.28 | 0.03 | 0.20 | 0.09 | 0.11 | 0.02 |
| Heart | 0.47 | 0.03 | 0.37 | 0.04 | 0.28 | 0.04 |
| Lung | 1.29 | 0.23 | 1.04 | 0.10 | 0.68 | 0.17 |
| Liver | 1.98 | 0.13 | 1.40 | 0.09 | 0.78 | 0.01 |
| Pancreas | 0.70 | 0.08 | 0.60 | 0.05 | 0.18 | 0.04 |
| Spleen | 0.69 | 0.03 | 0.55 | 0.04 | 0.25 | 0.01 |
| Adrenals | 1.47 | 0.34 | 0.80 | 0.08 | 0.42 | 0.04 |
| Kidney | 290.99 | 39.40 | 304.07 | 8.02 | 223.47 | 19.92 |
| Stomach | 0.65 | 0.21 | 0.55 | 0.05 | 0.53 | 0.09 |
| Small intestine | 0.57 | 0.09 | 0.52 | 0.09 | 0.28 | 0.04 |
| Large intestine | 0.88 | 0.10 | 0.71 | 0.08 | 0.35 | 0.01 |
| Ovary | 13.05 | 2.18 | 6.53 | 3.59 | 0.62 | 0.13 |
| Uterus | 1.25 | 0.05 | 1.29 | 0.15 | 0.65 | 0.14 |
| Skin | 0.95 | 0.22 | 1.02 | 0.66 | 0.74 | 0.34 |
| Muscle | 0.25 | 0.07 | 0.21 | 0.05 | 0.17 | 0.04 |
| Bone | 0.53 | 0.04 | 0.18 | 0.03 | 0.15 | 0.03 |
| Joint | 0.93 | 0.48 | 0.49 | 0.02 | 0.34 | 0.03 |
| Brain | 0.52 | 0.05 | 0.41 | 0.03 | 0.02 | 0.01 |

Data expressed as %IA/g and presented as mean ± SD (n = 3).

In the third part, the diagnostic potential of ¹³¹I-labeled, untagged VHHs A1 and A2 (SEQ ID NO 8 and 9, resp.) was assessed via SPECT/CT imaging in athymic nude mice bearing human FOLR1^{pos} SKOV-3 tumors. In this part the aim was to evaluate whether anti-human FOLR1 VHHs were able to specifically accumulate in human FOLR1^{pos} SKOV-3 tumors.

For this, mice were inoculated with human FOLR1^{pos} SKOV-3 tumor cells. After confirming the presence of tumors (size of 82.15 ± 52.98 mm³), mice were intravenously injected with ± 20 µg of ¹³¹I-labeled VHHs A1 and A2. Micro-SPECT/CT imaging was performed 2 h after tracer injection using a MILabs VECTor⁺/CT system and according to the procedure described in (D′Huyvetter et al. Clin Cancer Res. 2017 Nov 1;23(21):6616-6628.). In short, imaging was performed using a mouse PET collimator and a spiral scan mode of 94 bed positions (19 s per position). For CT, a normal scan mode of only one position was used. The obtained Micro-SPECT/CT data are reconstructed with a 0.6 voxel size, 2 subsets and 7 iterations, after which images are fused and corrected for attenuation based on the CT scan. Images are analyzed using a medical image data analysis tool (AMIDE). Uptake values in organs and tissues were analyzed and expressed as % injected activity per cm³ (%IA/cc).

The SPECT/CT imaging results (table 17) indicate that both ¹³¹I-labeled VHHs accumulate specifically in tumors and that the uptake in human FOLR1^{pos} SKOV-3 tumors is not significantly different between the two compounds two hours after tracer administration (unpaired t-test, p=0.3071). In addition, ¹³¹I-labeled VHH A1 is much less retained in kidneys (2.00 ± 0.56%lA/cc) compared to ¹³¹I-labeled VHH A2 (7.44 ± 4.16%lA/cc).

TABLE 17

Uptake values of the ¹³¹I-labeled VHH A1 and A2 in mice bearing human FOLR1^{pos} SKOV-3 tumors, expressed as %IA/cc

| %IA/cc 2h p.i. | ¹³¹I-VHH A1 | | ¹³¹I-VHH A2 | |
|---|---|---|---|---|
| Organ/tissue | Mean | SD | Mean | SD |
| Heart | 0.15 | 0.05 | 0.11 | 0.01 |
| Lung | 0.17 | 0.04 | 0.12 | 0.02 |
| Liver | 0.36 | 0.05 | 0.26 | 0.05 |
| Kidney | 2.00 | 0.56 | 7.44 | 4.16 |
| Muscle | 0.18 | 0.09 | 0.16 | 0.13 |
| Tumor | 1.88 | 0.44 | 2.36 | 0.56 |

Data are presented as mean ± SD (n = 3).

Taken together, this example indicates that imaging of human FOLR1-expression with different VHHs selective for human FOLR1 is feasible, both in organs that naturally express the target, as well as when expressed on tumor tissue, and this using diagnostic or theranostic radioisotopes. Importantly, we show here that the herein presented radiolabeled human FOLR1-targeting VHHs are able to selectively accumulate in the brain choroid plexus in the case of hetero- and homo-zygote human FOLR1 knock-in mice, but not in the brain choroid plexus of wildtype mice due to lack of crossreactivity with mouse FOLR1.

### Example 6: Therapeutic Use of the Labelled Compound

In this example we describe the therapeutic potential of several human FOLR1-targeting VHHs by investigating their therapeutic potential after radiolabelling with the therapeutic radionuclide ¹³¹I. Therapeutic potential is evaluated in vitro by means of their ability to bind human FOLR1-expressing cells (saturation binding and cellular retention) and in vivo by assessing their biodistribution in relevant mouse models and calculating the corresponding therapeutic index.

His₆-tagged VHHs A1, A2, A3, A4 (SEQ ID NO 53, 54, 55, 56; resp.) and untagged VHHs A1, A2, A3 (SEQ ID NO 8, 9, 24), were radiolabelled with the therapeutic radionuclide ¹³¹I using the SGMIB linker, according to the procedure described in example 3.

In a first part, their in vitro behaviour was assessed by means of investigating their cellular binding and retention over time. The binding potential of the resulting radioconjugates was assessed to confirm that this was not affected by the introduction of ¹³¹I-SGMIB into the amino acid sequence of the different VHHs. To this, human FOLR1^{pos} SKOV-3 cancer cells were incubated with serial dilutions with concentrations ranging from 0 to 300 nM of the different ¹³¹I-labelled, His₆ tagged VHHs. A 100-fold excess of the corresponding unlabeled VHH was added in parallel to saturate the human FOLR1 receptors expressed on cancer cells, to assess non-specific binding.

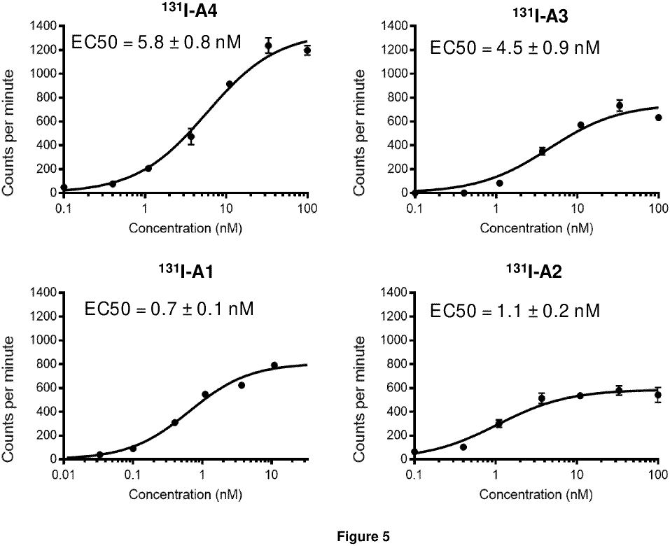

All ¹³¹I-labeled His₆-tagged VHHs showed dose-response curves in cell-binding experiments on human FOLR1^{pos} SKOV-3 cancer cells, indicating that ¹³¹I-labeling did not affect binding potential. The EC50 values of the four His₆-tagged human FOLR1 VHHs on human FOLR1^{pos} SKOV-3 cancer cells calculated 5.8 ± 0.8 nM (VHH A4), 4.5 ± 0.9 nM (VHH A3), 0.7 ± 0.1 (VHH A1) and 1.1 ± 0.2 nM (VHH A2), which agreed with the relative affinities determined in example 3 (FIG. 5).

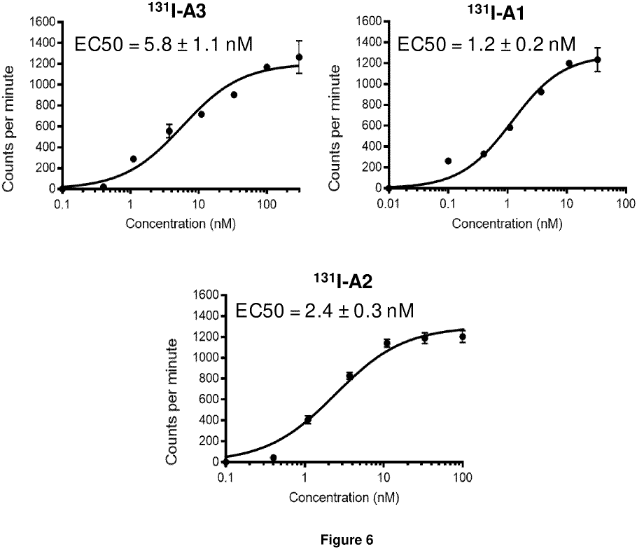

Also ¹³¹I-labeled, untagged VHHs A1, A2, A3, showed dose-response binding curves on human FOLR1^{pos} SKOV-3 cells, with calculated EC50 values of 5.8 ± 1.1 nM (VHH A3), 1.2 ± 0.3 (VHH A1) and 2.4 ± 0.3 nM (VHH A2). Again, these dose-response curved compared relatively with the affinities of the VHHs, as determined in example 4. This indicates that carboxyteminal hexahistidine-tags do not affect the human FOLR1-specific cell binding potential of the theranostic ¹³¹I-labeled compounds (FIG. 6).

The in vitro cellular retention of the different His₆- and untagged ¹³¹I-labeled VHHs was assessed on the same cancer cells. In this particular case, human FOLR1^{pos} SKOV-3 cancer cells were incubated with 10 nM ¹³¹I-labelled VHHs for 1h at 4° C., after which the unbound fraction was collected. A 100-fold excess of the corresponding unlabeled VHH to assess non-specific binding. Next, the cells were incubated with fresh medium up to 24 h at 37° C., after which the dissociated fraction was collected. Afterwards, the cells were washed with 0.05 M glycine pH 2.8 to collect the membrane-bound fraction. Finally, cells were solubilized with 1 M NaOH at room temperature to collect the internalized fraction. The sum of the membrane-bound and internalized fractions corresponds to the total cell-associated fraction.

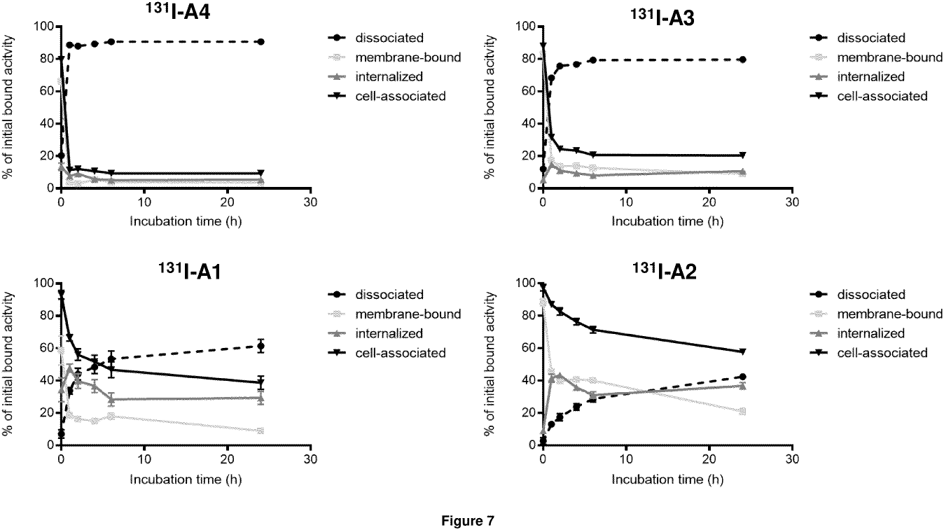

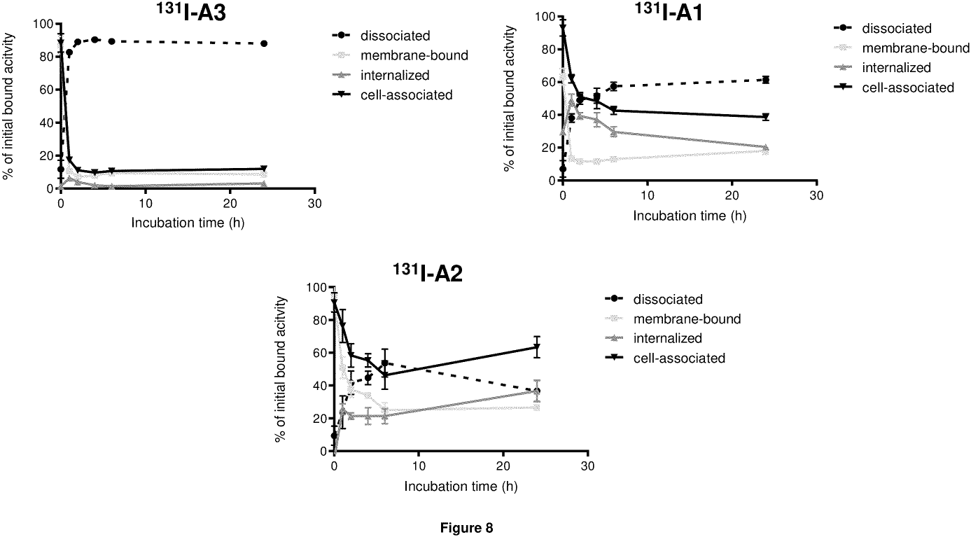

Cell-associated fraction was remarkably higher at all time points for ¹³¹I-labeled, His₆-tagged VHHs A1 and A2 compared to VHHs A3 and A4 (FIG. 7). After 24h, about 60% of initial bound activity was still cell-associated in the case of ¹³¹I-VHH A2 and about 40% for ¹³¹I-VHH A1 compared to only 10% for ¹³¹I-VHH A4 and 20% for ¹³¹I-VHH A3. This observation was confirmed for the ¹³¹I-labeled untagged VHHs, where cell-associated fraction was remarkably higher at all time points for ¹³¹I-VHH A1 and ¹³¹1-VHH A2 compared to ¹³¹I-VHH A3 (FIG. 8). Again, after 24h, about 60% of initial bound activity was still cell-associated in the case of ¹³¹I-VHH A2 and about 40% for ¹³¹I-VHH A1 compared to only 15% for ¹³¹I-VHH A3.

These in vitro observations are important, as they indicate first-hand the therapeutic potential of the described human FOLR1-targeting VHHs. Indeed, The remarkable subnanomolar binding affinity as described in example 4 and a cellular retention over 24h of about 40-60% of initial bound activity obtained for ¹³¹I-labeled VHHs A1 and A2 indicates that therapeutic cytotoxic radiation cancer be targeted to malignant cancer cells in a specific and sufficient manner.

In a second part, the biodistribution of ¹³¹I-labeled, untagged VHHs A1, A2, A3 was evaluated in mice at three time points after i.v. injection (3, 6 and 24 h p.i.). To this, athymic nude mice (n=3 per time point/per VHH) were inoculated subcutaneously with human FOLR1^{pos} SKOV-3 tumor cells in the right hind leg. After validation of tumor growth (about 100 mm³), all mice were intravenously injected in the tail vein with ± 5 µg of the different ¹³¹I-labeled VHHs. Next, the mice were euthanized by cervical dislocation after 3, 6 and 24 h post injection, dissected after which different organs and tissues were collected. Organs and tissues of interest were weighed and measured for radioactivity using an automatic gamma counter, along with injection standards. Results were expressed as % of injected activity (IA)/g tissue. The overall biodistribution of all three ¹³¹I-labeled, untagged VHHs was comparable, with low accumulation in normal organs and tissues (Table 18a-c). However, the highest uptake in tumor was observed for ¹³¹I-labeled VHH A1 at all time points, with uptake values of 7.61 ± 2.49, 5.72 ± 1.30 and 3.92 ± 2.22%IA/g after 3, 6 and 24 h compared to 2.87 ± 1.60, 1.95 ± 0.24 and 1.47 ± 1.19%IA/g for VHH A3 and 3.65 ± 2.59, 2.16 ± 0.21 and 2.53 ± 2.83%IA/g in the case of VHH A3. In addition, the same VHH is characterised by the most efficient clearance from kidneys, indicated by the lowest uptake values in that specific organ at all time points.

As a result, the most optimal therapeutic index (tumor-to-kidney uptake ratio) was obtained for ¹³¹I-labeled untagged VHH A1, as depicted in tables 18a-c and 19, with tumor-to-kidney values significantly higher compared to ¹³¹I-labeled untagged VHHs A2 and A3 at all time points (one-way ANOVA, multiple comparisons, p < 0.05).

TABLE 18a

uptake values in different organs and tissues of SKOV-3 tumor-bearing mice, for ¹³¹I-labeled, untagged VHH A1 at 3, 6 and 24 h post i.v. injection, expressed as %injected activity per gram of tissue (%IA/g)
¹³¹I-A1

| | 3 hpi | | 6 hpi | | 24 hpi | |
|---|---|---|---|---|---|---|
| Organ/tissue | Mean | SD | Mean | SD | Mean | SD |
| Blood | 0.04 | 0.002 | 0.02 | 0.003 | 0.008 | 0.0002 |
| Lymphnodes | 0.14 | 0.02 | 0.13 | 0.07 | / | / |
| Thyroid | 0.29 | 0.33 | 0.13 | 0.04 | 0.08 | 0.01 |
| Heart | 0.09 | 0.01 | 0.04 | 0.01 | 0.01 | 0.001 |
| Lung | 0.20 | 0.01 | 0.11 | 0.04 | 0.01 | 0.01 |
| Galbladder | 0.25 | 0.12 | 0.21 | 0.15 | 0.17 | 0.12 |
| Liver | 0.30 | 0.01 | 0.13 | 0.02 | 0.02 | 0.001 |
| Pancreas | 0.05 | 0.005 | 0.03 | 0.01 | 0.005 | 0.0003 |
| Spleen | 0.11 | 0.01 | 0.06 | 0.01 | 0.01 | 0.002 |
| Adrenals | 0.09 | 0.01 | 0.03 | 0.01 | 0.01 | 0.01 |
| Kidney | 1.93 | 0.20 | 0.51 | 0.11 | 0.08 | 0.01 |
| Stomach | 0.17 | 0.08 | 0.12 | 0.09 | 0.01 | 0.002 |
| Small intestine | 0.14 | 0.11 | 0.14 | 0.09 | 0.02 | 0.01 |
| Large intestine | 0.12 | 0.02 | 0.11 | 0.06 | 0.02 | 0.01 |
| Uterus | 0.17 | 0.03 | 0.08 | 0.01 | 0.02 | 0.01 |
| Skin | 0.27 | 0.02 | 0.30 | 0.22 | 0.06 | 0.02 |
| Muscle | 0.04 | 0.01 | 0.03 | 0.004 | 0.01 | 0.005 |
| Bone | 0.05 | 0.04 | 0.03 | 0.004 | 0.01 | 0.001 |
| Tumor | 7.61 | 2.49 | 5.72 | 1.30 | 3.92 | 2.22 |
| Brain | 0.007 | 0.0005 | 0.007 | 0.001 | 0.002 | 0.001 |
| Therapeutic index | 1.03 | 1.70 | 11.30 | 0.28 | 48.98 | 21.50 |

Therapeutic index defined as tumor-to-kidney uptake ratio.
Data presented as mean ± SD (n = 3).

TABLE 18b

uptake values in different organs and tissues of SKOV-3 tumor-bearing mice, for ¹³¹I-labeled, untagged VHH A2 at 3, 6 and 24 h post i.v. injection, expressed as %injected activity per gram of tissue (%IA/g)
¹³¹I-A2

| | 3 hpi | | 6 hpi | | 24 hpi | |
|---|---|---|---|---|---|---|
| Organ/tissue | Mean | SD | Mean | SD | Mean | SD |
| Blood | 0.04 | 0.01 | 0.02 | 0.001 | 0.01 | 0.0004 |
| Lymphnodes | 0.54 | 0.03 | 0.23 | 0.04 | 0.06 | 0.02 |
| Thyroid | / | / | 0.43 | 0.02 | 0.64 | 0.74 |
| Heart | 0.07 | 0.01 | 0.04 | 0.01 | 0.01 | 0.002 |
| Lung | 0.17 | 0.01 | 0.05 | 0.01 | 0.01 | 0.0002 |
| Galbladder | 0.37 | 0.13 | 0.06 | 0.05 | 0.05 | 0.04 |
| Liver | 0.24 | 0.03 | 0.10 | 0.02 | 0.02 | 0.003 |
| Pancreas | 0.05 | 0.004 | 0.03 | 0.01 | 0.01 | 0.002 |
| Spleen | 0.09 | 0.01 | 0.05 | 0.01 | 0.01 | 0.002 |
| Adrenals | 0.13 | 0.03 | 0.04 | 0.01 | 0.02 | 0.01 |
| Kidney | 6.75 | 2.16 | 1.60 | 0.41 | 0.29 | 0.03 |
| Stomach | 0.16 | 0.12 | 0.05 | 0.02 | 0.01 | 0.001 |
| Small intestine | 0.26 | 0.24 | 0.08 | 0.03 | 0.01 | 0.01 |
| Large intestine | 0.10 | 0.01 | 0.08 | 0.02 | 0.01 | 0.001 |
| Uterus | 0.13 | 0.04 | 0.06 | 0.01 | 0.01 | 0.003 |
| Skin | 0.44 | 0.16 | 0.19 | 0.03 | 0.07 | 0.04 |
| Muscle | 0.07 | 0.02 | 0.03 | 0.01 | 0.01 | 0.002 |
| Bone | 0.06 | 0.01 | 0.03 | 0.01 | 0.01 | 0.002 |
| Tumor | 3.65 | 2.59 | 2.16 | 0.21 | 2.53 | 2.83 |
| Brain | 0.01 | 0.002 | 0.01 | 0.001 | 0.003 | 0.002 |
| Therapeutic index | 0.57 | 0.46 | 1.42 | 0.42 | 9.23 | 10.90 |

Therapeutic index defined as tumor-to-kidney uptake ratio.
Data presented as mean ± SD (n = 3).

TABLE 18

cuptake values in different organs and tissues of SKOV-3 tumor-bearing mice, for ¹³¹I-labeled, untagged VHH A3 at 3, 6 and 24 h post i.v. injection, expressed as %injected activity per gram of tissue (%IA/g)
¹³¹I-A3

| | 3 hpi | | 6 hpi | | 24 hpi | |
|---|---|---|---|---|---|---|
| Organ/tissue | Mean | SD | Mean | SD | Mean | SD |
| Blood | 0.03 | 0.005 | 0.01 | 0.003 | 0.004 | 0.001 |
| Lymphnodes | 0.29 | 0.07 | 0.19 | 0.08 | 0.18 | 0.08 |
| Thyroid | 0.66 | 0.13 | 0.46 | 0.26 | 1.22 | 0.55 |
| Heart | 0.07 | 0.01 | 0.04 | 0.002 | 0.01 | 0.001 |
| Lung | 0.12 | 0.03 | 0.06 | 0.01 | 0.01 | 0.002 |
| Galbladder | 0.17 | 0.01 | 0.09 | 0.01 | 0.48 | 0.56 |
| Liver | 0.16 | 0.04 | 0.08 | 0.01 | 0.01 | 0.003 |
| Pancreas | 0.06 | 0.01 | 0.03 | 0.01 | 0.01 | 0.002 |
| Spleen | 0.06 | 0.01 | 0.04 | 0.01 | 0.01 | 0.01 |
| Adrenals | 0.10 | 0.02 | 0.04 | 0.01 | 0.03 | 0.01 |
| Kidney | 3.59 | 0.58 | 0.72 | 0.19 | 0.07 | 0.01 |
| Stomach | 0.10 | 0.05 | 0.07 | 0.05 | 0.03 | 0.04 |
| Small intestine | 0.08 | 0.01 | 0.05 | 0.02 | 0.01 | 0.003 |
| Large intestine | 0.11 | 0.04 | 0.07 | 0.02 | 0.02 | 0.01 |
| Uterus | 0.12 | 0.01 | 0.05 | 0.01 | 0.03 | 0.02 |
| Skin | 0.27 | 0.04 | 0.19 | 0.02 | 0.08 | 0.03 |
| Muscle | 0.06 | 0.02 | 0.04 | 0.01 | 0.02 | 0.01 |
| Bone | 0.06 | 0.01 | 0.04 | 0.01 | 0.03 | 0.01 |
| Tumor | 2.87 | 1.60 | 1.95 | 0.24 | 1.47 | 1.19 |
| Brain | 0.02 | 0.01 | 0.01 | 0.002 | 0.002 | 0.001 |
| Therapeutic index | 0.80 | 0.45 | 2.82 | 0.52 | 24.05 | 22.11 |

Therapeutic index defined as tumor-to-kidney uptake ratio.
Data presented as mean ± SD (n = 3)

TABLE 19

therapeutic index (tumor-to-kidney uptake ratio) for ¹³¹I-labeled, untagged VHHs A1, A2 and A3 at 3, 6 and 24 h post i.v. injection

| Therapeutic index | 3 h | 6 h | 24 h |
|---|---|---|---|
| A3 | 0.80 ± 0.45 | 2.82 ± 0.52 | 24.05 ± 22.11 |
| A1 | 4.03 ± 1.70 | 11.30 ± 0.28 | 48.98 ± 21.50 |
| A2 | 0.57 ± 0.46 | 1.42 ± 0.42 | 9.23 ± 10.90 |

Data presented as mean ± SD (n = 3).

Next, the long-term biodistribution and tumor targeting potential of ¹³¹I-labeled, untagged VHHs A1 and A2 was evaluated in mice with human FOLR1^{pos} SKOV-3 tumors over 6 days post i.v. injection. To this, athymic nude mice (n=3 per time point/per VHH) were inoculated subcutaneously with human FOLR1^{pos} SKOV-3 tumor cells in the right hind leg. After validation of tumor growth (size of 82.15 ± 52.98 mm³), all mice were intravenously injected in the tail vein with ± 15 uCi (± 5 µg) of the different ¹³¹I-labeled, untagged VHHs. Next, the mice were euthanized by cervical dislocation up to 144 h post injection, dissected after which different organs and tissues were collected. Organs and tissues of interest were weighed and measured for radioactivity using an automatic gamma counter, along with injection standards. Results were expressed as % of injected activity (IA)/g tissue.

Intravenous injection of ¹³¹I-labelled, untagged VHH A1 reveals higher tumor uptake and retention compared to ¹³¹I-labelled, untagged VHH A2. Also, the retention of ¹³¹I-labelled, untagged VHH A1 in kidneys was measured lower compared to ¹³¹I-labelled, untagged VHH A2 at all time points. For example, after 1h the amount retained in kidneys measures 26.30 ± 8.56%IA/g for ¹³¹I-VHH A1 compared to 54.56 ± 13.21%IA/g for ¹³¹I-VHH A2, a value that is almost double at the same time point after injection (tables 20a-c and 21a-c). This difference is also reflected in the calculated therapeutic index (tumor-to-kidney ratio), which are significantly higher for ¹³¹I-VHH A1 compared to ¹³¹I-VHH A2 at all time points (unpaired t-test, p < 0.05), as depicted in table 22.

TABLE 20a

uptake values in different organs and tissues of SKOV-3 tumor-bearing mice, for ¹³¹I-labeled, untagged VHH A1 up to 6 h post i.v. injection, expressed as %injected activity per gram of tissue (%IA/g)

| | 1 h | | 3 h | | 6 h | |
|---|---|---|---|---|---|---|
| Organ/tissue | Mean | SD | Mean | SD | Mean | SD |
| Blood | 0.27 | 0.05 | 0.04 | 0.003 | 0.02 | 0.001 |
| Lymphnodes | 0.59 | 0.34 | 0.27 | 0.07 | 0.20 | 0.01 |
| Thyroid (%IA) | 0.001 | 0.0004 | 0.0004 | 0.0003 | 0.0002 | 0.00004 |
| Heart | 0.27 | 0.03 | 0.08 | 0.01 | 0.05 | 0.01 |
| Lung | 0.67 | 0.10 | 0.21 | 0.02 | 0.12 | 0.05 |
| Liver | 0.92 | 0.08 | 0.51 | 0.04 | 0.25 | 0.04 |
| Pancreas | 0.16 | 0.03 | 0.06 | 0.01 | 0.03 | 0.002 |
| Spleen | 0.25 | 0.03 | 0.15 | 0.02 | 0.11 | 0.02 |
| Adrenals | 0.82 | 0.38 | 0.12 | 0.02 | 0.06 | 0.01 |
| Kidney | 26.30 | 8.56 | 1.26 | 0.13 | 0.55 | 0.12 |
| Stomach | 0.35 | 0.08 | 0.11 | 0.03 | 0.06 | 0.01 |
| Small intestine | 0.41 | 0.07 | 0.12 | 0.04 | 0.05 | 0.01 |
| Large intestine | 0.38 | 0.05 | 0.13 | 0.02 | 0.08 | 0.003 |
| Uterus | 0.50 | 0.18 | 0.18 | 0.03 | 0.10 | 0.02 |
| Skin | 0.49 | 0.14 | 0.19 | 0.003 | 0.11 | 0.02 |
| Muscle | 0.21 | 0.14 | 0.06 | 0.01 | 0.03 | 0.003 |
| Bone | 0.24 | 0.11 | 0.07 | 0.01 | 0.04 | 0.003 |
| Tumor | 6.32 | 1.30 | 6.02 | 0.95 | 4.70 | 0.57 |
| Brain | 0.05 | 0.02 | 0.01 | 0.003 | 0.01 | 0.001 |
| Therapeutic index | 0.25 | 0.04 | 4.87 | 1.19 | 8.93 | 2.68 |

Therapeutic index defined as tumor-to-kidney uptake ratio.
Data presented as mean ± SD (n = 3).

TABLE 20b

uptake values in different organs and tissues of SKOV-3 tumor-bearing mice, for ¹³¹I-labeled, untagged VHH A1 from 24 h up to 72 h post i.v. injection, expressed as %injected activity per gram of tissue (%IA/g)

| | 24 h | | 48 h | | 72 h | |
|---|---|---|---|---|---|---|
| Organ/tissue | Mean | SD | Mean | SD | Mean | SD |
| Blood | 0.01 | 0.001 | 0.002 | 0.001 | 0.002 | 0.001 |
| Lymphnodes | 0.09 | 0.02 | 0.02 | 0.01 | 0.03 | 0.03 |
| Thyroid (%IA) | 0.0001 | 0.000001 | 0.001 | 0.001 | 0.001 | 0.001 |
| Heart | 0.01 | 0.001 | 0.003 | 0.001 | 0.002 | 0.001 |
| Lung | 0.02 | 0.01 | 0.01 | 0.01 | 0.01 | 0.003 |
| Liver | 0.03 | 0.002 | 0.01 | 0.001 | 0.01 | 0.003 |
| Pancreas | 0.01 | 0.001 | 0.002 | 0.0001 | 0.001 | 0.0002 |
| Spleen | 0.02 | 0.003 | 0.01 | 0.001 | 0.005 | 0.001 |
| Adrenals | 0.01 | 0.01 | 0.01 | 0.01 | 0.01 | 0.01 |
| Kidney | 0.08 | 0.01 | 0.03 | 0.01 | 0.02 | 0.003 |
| Stomach | 0.01 | 0.001 | 0.01 | 0.003 | 0.01 | 0.001 |
| Small intestine | 0.01 | 0.003 | 0.004 | 0.001 | 0.002 | 0.001 |
| Large intestine | 0.02 | 0.004 | 0.01 | 0.004 | 0.01 | 0.004 |
| Uterus | 0.04 | 0.02 | 0.005 | 0.002 | 0.01 | 0.01 |
| Skin | 0.03 | 0.005 | 0.01 | 0.002 | 0.004 | 0.002 |
| Muscle | 0.01 | 0.003 | 0.003 | 0.002 | 0.01 | 0.01 |
| Bone | 0.02 | 0.01 | 0.02 | 0.001 | 0.01 | 0.001 |
| Tumor | 2.98 | 0.78 | 1.08 | 0.31 | 0.22 | 0.06 |
| Brain | 0.003 | 0.001 | 0.001 | 0.0005 | 0.001 | 0.0003 |
| Therapeutic index | 41.26 | 18.16 | 36.81 | 14.57 | 13.94 | 6.05 |

Therapeutic index defined as tumor-to-kidney uptake ratio.
Data presented as mean ± SD (n = 3).

TABLE 20c

uptake values in different organs and tissues of SKOV-3 tumor-bearing mice, for ¹³¹I-labeled, untagged VHH A1 from 96 h up to 144 h post i.v. injection, expressed as %injected activity per gram of tissue (%IA/g)

| | 96 h | | 120 h | | 144 h | |
|---|---|---|---|---|---|---|
| Organ/tissue | Mean | SD | Mean | SD | Mean | SD |
| Blood | 0.002 | 0.0002 | 0.002 | 0.0003 | 0.002 | 0.0002 |
| Lymphnodes | 0.08 | 0.04 | 0.002 | 0.0005 | 0.08 | 0.01 |
| Thyroid (%IA) | 0.002 | 0.002 | 0.00002 | / | 0.0003 | 0.0001 |
| Heart | 0.002 | 0.001 | 0.002 | 0.001 | 0.003 | 0.001 |
| Lung | 0.01 | 0.001 | 0.002 | 0.0005 | 0.002 | 0.002 |
| Liver | 0.01 | 0.002 | 0.004 | 0.001 | 0.01 | 0.001 |
| Pancreas | 0.001 | 0.001 | 0.001 | 0.0003 | 0.001 | 0.001 |
| Spleen | 0.001 | 0.0003 | 0.003 | 0.001 | 0.01 | 0.0005 |
| Adrenals | 0.01 | / | 0.01 | / | 0.01 | 0.003 |
| Kidney | 0.02 | 0.002 | 0.01 | 0.01 | 0.01 | 0.001 |
| Stomach | 0.01 | 0.01 | 0.01 | 0.01 | 0.01 | 0.01 |
| Small intestine | 0.001 | 0.0004 | 0.002 | 0.002 | 0.01 | 0.01 |
| Large intestine | 0.005 | 0.001 | 0.002 | 0.001 | 0.01 | 0.01 |
| Uterus | 0.01 | 0.01 | 0.003 | 0.001 | 0.01 | 0.01 |
| Skin | 0.003 | 0.002 | 0.002 | 0.001 | 0.01 | 0.01 |
| Muscle | 0.01 | 0.004 | 0.001 | 0.001 | 0.01 | / |
| Bone | 0.02 | 0.01 | 0.004 | 0.003 | 0.002 | 0.001 |
| Tumor | 0.21 | 0.05 | 0.10 | 0.04 | 0.06 | 0.02 |
| Brain | 0.0003 | 0.0002 | 0.002 | 0.001 | 0.001 | 0.001 |
| Therapeutic index | 13.68 | 4.59 | 24.62 | 30.01 | 8.53 | 2.92 |

Therapeutic index defined as tumor-to-kidney uptake ratio.
Data presented as mean ± SD (n = 3).

TABLE 21a

uptake values in different organs and tissues of SKOV-3 tumor-bearing mice, for ¹³¹I-labeled, untagged VHH A2 up to 6 h post i.v. injection, expressed as %injected activity per gram of tissue (%IA/g)

| | 1 h | | 3 h | | 6 h | |
|---|---|---|---|---|---|---|
| Organ/tissue | Mean | SD | Mean | SD | Mean | SD |
| Blood | 0.30 | 0.10 | 0.04 | 0.004 | 0.03 | 0.01 |
| Lymphnodes | 0.55 | 0.32 | 0.21 | 0.03 | 0.18 | 0.04 |
| Thyroid (%IA) | 0.001 | 0.001 | 0.0004 | 0.0002 | 0.001 | 0.001 |
| Heart | 0.24 | 0.04 | 0.06 | 0.01 | 0.04 | 0.01 |
| Lung | 0.41 | 0.07 | 0.13 | 0.02 | 0.10 | 0.01 |
| Liver | 0.52 | 0.02 | 0.23 | 0.02 | 0.15 | 0.04 |
| Pancreas | 0.16 | 0.09 | 0.04 | 0.003 | 0.03 | 0.01 |
| Spleen | 0.20 | 0.02 | 0.10 | 0.01 | 0.07 | 0.01 |
| Adrenals | 0.75 | 0.24 | 0.11 | 0.01 | 0.05 | 0.01 |
| Kidney | 54.56 | 13.21 | 5.71 | 1.92 | 2.02 | 0.54 |
| Stomach | 0.35 | 0.10 | 0.10 | 0.01 | 0.10 | 0.03 |
| Small intestine | 0.30 | 0.06 | 0.08 | 0.01 | 0.06 | 0.01 |
| Large intestine | 0.25 | 0.07 | 0.09 | 0.03 | 0.10 | 0.08 |
| Uterus | 0.36 | 0.12 | 0.09 | 0.02 | 0.07 | 0.01 |
| Skin | 0.44 | 0.14 | 0.18 | 0.02 | 0.21 | 0.12 |
| Muscle | 0.15 | 0.04 | 0.05 | 0.01 | 0.04 | 0.01 |
| Bone | 0.16 | 0.06 | 0.09 | 0.06 | 0.04 | 0.01 |
| Tumor | 3.85 | 1.88 | 3.26 | 1.20 | 3.22 | 0.89 |
| Brain | 0.02 | 0.005 | 0.01 | 0.005 | 0.01 | 0.004 |
| Therapeutic index | 0.07 | 0.02 | 0.62 | 0.33 | 1.66 | 0.58 |

Therapeutic index defined as tumor-to-kidney uptake ratio.
Data presented as mean ± SD (n = 3).

TABLE 21b

uptake values in different organs and tissues of SKOV-3 tumor-bearing mice, for ¹³¹I-labeled, untagged VHH A2 from 24 h up to 72 h post i.v. injection, expressed as %injected activity per gram of tissue (%IA/g)

| | 24 h | | 48 h | | 72 h | |
|---|---|---|---|---|---|---|
| Organ/tissue | Mean | SD | Mean | SD | Mean | SD |
| Blood | 0.01 | 0.001 | 0.002 | 0.001 | 0.002 | 0.001 |
| Lymphnodes | 0.05 | 0.02 | 0.003 | 0.001 | 0.02 | 0.01 |
| Thyroid (%IA) | 0.004 | 0.004 | 0.01 | 0.01 | 0.0002 | 0.0002 |
| Heart | 0.01 | 0.002 | 0.002 | 0.001 | 0.003 | 0.001 |
| Lung | 0.01 | 0.003 | 0.003 | 0.0003 | 0.01 | 0.002 |
| Liver | 0.02 | 0.002 | 0.01 | 0.001 | 0.01 | 0.0002 |
| Pancreas | 0.004 | 0.0003 | 0.001 | 0.0007 | 0.001 | 0.0002 |
| Spleen | 0.01 | 0.003 | 0.005 | 0.001 | 0.004 | 0.0004 |
| Adrenals | 0.02 | 0.003 | 0.01 | 0.003 | 0.01 | 0.006 |
| Kidney | 0.34 | 0.03 | 0.09 | 0.01 | 0.08 | 0.01 |
| Stomach | 0.01 | 0.002 | 0.01 | 0.009 | 0.005 | 0.003 |
| Small intestine | 0.01 | 0.003 | 0.01 | 0.004 | 0.005 | 0.004 |
| Large intestine | 0.01 | 0.004 | 0.005 | 0.002 | 0.004 | 0.002 |
| Uterus | 0.02 | 0.01 | 0.003 | 0.002 | 0.003 | 0.001 |
| Skin | 0.04 | 0.01 | 0.01 | 0.01 | 0.005 | 0.001 |
| Muscle | 0.02 | 0.01 | 0.001 | 0.0005 | 0.003 | 0.001 |
| Bone | 0.01 | 0.003 | 0.002 | 0.001 | 0.002 | 0.001 |
| Tumor | 1.94 | 0.81 | 0.48 | 0.15 | 0.39 | 0.12 |
| Brain | 0.002 | 0.001 | 0.001 | 0.0003 | 0.0003 | 0.0002 |
| Therapeutic index | 5.65 | 2.00 | 5.48 | 2.36 | 4.79 | 1.16 |

Therapeutic index defined as tumor-to-kidney uptake ratio.
Data presented as mean ± SD (n = 3)

TABLE 21c

uptake values in different organs and tissues of SKOV-3 tumor-bearing mice, for ¹³¹I-labeled, untagged VHH A2 from 96 h up to 144 h post i.v. injection, expressed as %injected activity per gram of tissue (%IA/g)

| | 96 h | | 120 h | | 144 h | |
|---|---|---|---|---|---|---|
| Organ/tissue | Mean | SD | Mean | SD | Mean | SD |
| Blood | 0.002 | 0.0002 | 0.001 | 0.0003 | 0.001 | 0.0003 |
| Lymphnodes | 0.02 | 0.01 | 0.02 | 0.003 | 0.02 | 0.005 |
| Thyroid (%IA) | 0.0007 | 0.0009 | 0.003 | 0.003 | 0.001 | 0.0002 |
| Heart | 0.002 | 0.0005 | 0.001 | 0.0003 | 0.001 | 0.001 |
| Lung | 0.002 | 0.001 | 0.002 | 0.002 | 0.001 | 0.001 |
| Liver | 0.01 | 0.002 | 0.01 | 0.001 | 0.01 | 0.001 |
| Pancreas | 0.0005 | 0.0004 | 0.001 | 0.001 | 0.001 | 0.0004 |
| Spleen | 0.003 | 0.001 | 0.002 | 0.0003 | 0.002 | 0.001 |
| Adrenals | 0.01 | 0.003 | 0.01 | 0.005 | 0.002 | 0.002 |
| Kidney | 0.06 | 0.01 | 0.05 | 0.02 | 0.04 | 0.01 |
| Stomach | 0.005 | 0.002 | 0.002 | 0.0002 | 0.002 | 0.001 |
| Small intestine | 0.01 | 0.004 | 0.002 | 0.001 | 0.002 | 0.001 |
| Large intestine | 0.004 | 0.001 | 0.004 | 0.0002 | 0.003 | 0.001 |
| Uterus | 0.004 | 0.003 | 0.01 | 0.004 | 0.002 | 0.002 |
| Skin | 0.005 | 0.002 | 0.01 | 0.003 | 0.002 | 0.0004 |
| Muscle | 0.002 | 0.002 | 0.003 | 0.001 | 0.002 | 0.001 |
| Bone | / | / | 0.003 | 0.002 | 0.002 | 0.001 |
| Tumor | 0.27 | 0.17 | 0.13 | 0.13 | 0.07 | 0.01 |
| Brain | 0.0004 | 0.0002 | 0.0002 | 0.0001 | 0.001 | 0.00003 |
| Therapeutic index | 4.61 | 3.40 | 2.14 | 1.56 | 1.84 | 0.44 |

Therapeutic index defined as tumor-to-kidney uptake ratio.
Data presented as mean ± SD (n = 3).

TABLE 22

therapeutic index (tumor-to-kidney ratio) for ¹³¹ I-labeled, untagged VHHs A1 and A2 over 6 days post i.v. injection

| Therapeutic index | A1 | A2 |
|---|---|---|
| 1h | 0.25 ± 0.04 | 0.07 ± 0.02 |
| 3h | 4.87 ± 1.19 | 0.62 ± 0.33 |
| 6h | 8.93 ± 2.68 | 1.66 ± 0.58 |
| 24h | 41.26 ± 18.16 | 5.65 ± 2.00 |
| 48h | 36.81 ± 14.57 | 5.48 ± 2.36 |
| 72h | 13.94 ± 6.05 | 4.79 ± 1.16 |
| 96h | 13.68 ± 4.59 | 4.61 ± 3.40 |
| 120h | 24.62 ± 30.01 | 2.14 ± 1.56 |
| 144h | 8.53 ± 2.92 | 1.84 ± 0.44 |

Data presented as mean ± SD (n = 3).

The uptake values over 6 days post i.v. injection obtained from the long-term biodistribution of ¹³¹I-labeled untagged VHHs A1 and A2 were used as input data to calculate the mean absorbed dose (expressed as centi-gray per millicurie, cGy/mCi) to different organs and tissues after injection of 1 mCi of ¹³¹I-VHH A1 and ¹³¹I-VHH A1, which was compared to the values obtained for ¹³¹I-labeled HER2-targeting VHH 2Rs15d (referred to as CAM-H2) in a similar animal model and described in (D′Huyvetter et al. Clin Cancer Res. 2017 Nov 1;23(21):6616-6628.; and U.S. Pat. no.: 9855348). To obtain the mean absorbed dose to tissues and organs, the biodistribution data were time-integrated to obtain the residence time per gram tissue. Briefly, the area under the curve between 0 and 144 h was made using the trapezoid integration method. Next, the absorbed doses were calculated using S values for ¹³¹I obtained from RADAR phantoms (Unit Density Spheres). The S value for a 1 g sphere (0.0304 Gy.g/MBq.s) was used to calculate all organ doses.

Importantly, the lowest absorbed dose to kidneys was obtained for ¹³¹I-labeled untagged VHH A1, which was 2.7 times lower compared to the value obtained for ¹³¹I-labeled untagged VHHs A2 and even 5.3 times lower compared to CAM-H2 (¹³¹I-2Rs15d). In addition, therapeutic index obtained for ¹³¹I-VHH A1 calculated 4.43 which is a multiple of what has obtained for ¹³¹I-VHH A2 and CAM-H2, as depicted in table 23

TABLE 23

Dosimetry estimations for 1mCi of anti-HER2 untagged VHH 2Rs15d and anti-human FOLR1 untagged VHHs A1 and A2, all radiolabeled with ¹³¹I

| Organ/tissue | 2Rs15d | A1 | A2 |
|---|---|---|---|
| Brain | 9.38 | 1.45 | 1.01 |
| Lungs | 26.66 | 14.85 | 9.78 |
| Heart | 11.50 | 5.90 | 4.96 |
| Liver | 31.13 | 25.86 | 15.40 |
| Kidneys | 835.96 | 157.70 | 422.68 |
| Stomach | 24.94 | 10.31 | 9.77 |
| Pancreas | 3.75 | 3.92 | 3.06 |
| Spleen | 13.22 | 11.63 | 7.11 |
| Skin | 28.38 | 13.47 | 18.95 |
| Muscle | 11.37 | 6.40 | 5.49 |
| Bone | 32.24 | 10.43 | 4.89 |
| Small intestines | 20.79 | 7.21 | 8.15 |
| Large intestines | 15.79 | 11.07 | 8.82 |
| Lymphnodes | 13.11 | 37.99 | 23.35 |
| Blood | 14.72 | 3.90 | 4.47 |
| Uterus | 23.90 | 14.71 | 9.09 |
| Thyroid | 0.15 | 0.43 | 1.79 |
| Adenals | 0.19 | 0.14 | 0.19 |
| Galbladder | 0.18 | 0.10 | 0.11 |
| Tumor | 1188.34 | 699.15 | 477.29 |
| Tumor-to-kidney ratio | 2.55 | 4.43 | 1.13 |

Data are expressed as cGy/mCi

Taken together, this example indicates that targeting of human FOLR1-expression in vitro on cells and in vivo in tumors is feasible with different therapeutic radiolabeled VHHs selective for human FOLR1. Importantly, ¹³¹I-labeled A1 reveals an optimal therapeutic index that is a multiple of the therapeutic index of ¹³¹I-labeled A2 and also of that obtained for CAM-H2, a ¹³¹I-labeled anti-HER2 VHH that has shown to be therapeutically potent in similar animal models. As a consequence, we expect the therapeutic potential to be at least as outspoken as observed for CAM-H2.

### Example 7: Targeting Potential of 177Lu- and 225Ac-Labeled A1 and A2

### Example 7a:

Anti-human FOLR1 VHHs A1 and A2 (SEQ ID NO 8 and 9) were successfully radiolabeled with 177Lu, after which their biodistribution was assessed in athymic mice bearing human FOLR1^{pos} SKOV-3 tumors. To this, athymic nude mice (n=3 per VHH) were inoculated subcutaneously with human FOLR1^{pos} SKOV-3 tumor cells in the neck. After validation of tumor growth (size of about 100 mm³), all mice were intravenously injected in the tail vein with ± 5 µg of the different ¹⁷⁷Lu-DOTA-VHHs. Next, the mice were euthanized by cervical dislocation 1h post injection, dissected after which different organs and tissues were collected. Organs and tissues of interest were weighed and measured for radioactivity using an automatic gamma counter, along with injection standards. Results were expressed as % of injected activity (IA)/g tissue.

Intravenous injection of ¹⁷⁷Lu-DOTA-VHHs A1 and A2 reveals tumor uptake that is comparable to what is observed for their ¹³¹I-SGMIB-labeled variants (table 24). Uptake in additional organs and tissues was low (< 1.5% IA/g tissue) except for kidneys, which is the route for excretion of unbound radiolabeled VHHs. These results indicate that the human FOLR1 targeting potential of VHH A1 is not compromised after conjugation to 177Lu-DOTA.

TABLE 24

Uptake in different organs and tissues obtained from dissections 1 h after i.v. injection of ¹⁷⁷Lu-labeled anti-human FOLR1 VHHs in athymic mice bearing human FOLR1^{pos} SKOV-3 tumors

| Orqan/tissue | ¹⁷⁷Lu-A1 | | ¹⁷⁷Lu-A2 | |
|---|---|---|---|---|
| | MEAN | SD | MEAN | SD |
| Blood | 0.24 | 0.04 | 0.17 | 0.02 |
| Lymphnodes | 1.17 | 0.38 | 0.81 | 0.40 |
| Heart | 0.18 | 0.02 | 0.12 | 0.03 |
| Lung | 0.69 | 0.05 | 0.54 | 0.13 |
| Galbladder | 0.95 | 0.30 | 0.35 | 0.09 |
| Liver | 0.66 | 0.06 | 0.25 | 0.03 |
| Pancreas | 0.17 | 0.04 | 0.15 | 0.09 |
| Spleen | 0.27 | 0.05 | 0.13 | 0.01 |
| Adrenals | 1.48 | 0.17 | 0.87 | 0.25 |
| Kidney | 246.62 | 19.95 | 179.51 | 11.78 |
| Stomach | 0.55 | 0.13 | 0.39 | 0.13 |
| Small intestine | 0.35 | 0.01 | 0.23 | 0.09 |
| Large intestine | 0.40 | 0.07 | 0.26 | 0.10 |
| Uterus | 0.51 | 0.14 | 0.32 | 0.07 |
| Skin | 0.81 | 0.09 | 0.82 | 0.40 |
| Muscle | 0.23 | 0.04 | 0.20 | 0.07 |
| Bone | 0.44 | 0.24 | 0.17 | 0.05 |
| Tumor | 10.16 | 1.52 | 7.68 | 5.31 |
| Brain | 0.08 | 0.05 | 0.04 | 0.01 |

Data expressed as %IA/g and presented as mean ± SD (n = 3).

### Example 7b:

Anti-human FOLR1 VHH A1 (SEQ ID NO: 8) was successfully radiolabeled with 225Ac, after which its biodistribution was assessed in athymic mice bearing human FOLR1^{pos} SKOV-3 tumors. To this, athymic nude mice (n=3) were inoculated subcutaneously with human FOLR1^{pos} SKOV-3 tumor cells in the neck. After validation of tumor growth (size of about 100 mm³), all mice were intravenously injected in the tail vein with ± 5 µg of ²²⁵Ac-DOTA-VHH A1. Next, the mice were euthanized by cervical dislocation 1h post injection, dissected after which different organs and tissues were collected. Organs and tissues of interest were weighed and measured for radioactivity using an automatic gamma counter, along with injection standards. Results were expressed as % of injected activity (IA)/g tissue.

Intravenous injection of ²²⁵Ac-DOTA-VHH A1 reveals tumor uptake that is comparable to what is observed for the ¹³¹I-SGMIB- and ¹⁷⁷Lu-DOTA-labeled variants of VHH A1 (table 25). Uptake in additional organs and tissues was low (< 1.5% IA/g tissue) except for kidneys, which is the route for excretion of unbound radiolabeled VHHs. These results indicate that the human FOLR1 targeting potential of VHH A1 is not compromised after conjugation to 225Ac-DOTA.

TABLE 25

Uptake in different organs and tissues obtained from dissections 1 h after i.v. injection of ¹⁷⁷Lu-labeled anti-human FOLR1 VHH A1 in athymic mice bearing human FOLR1^{pos} SKOV-3 tumors

| Organ/tissue | 225Ac-A1 | |
|---|---|---|
| | Mean | SD |
| Blood | 0.26 | 0.15 |
| Heart | 0.26 | 0.06 |
| Lung | 0.71 | 0.12 |
| Liver | 0.65 | 0.11 |
| Pancreas | 0.26 | 0.05 |
| Spleen | 0.33 | 0.02 |
| Adrenals | 2.86 | 1.75 |
| Kidneys | 185.42 | 30.48 |
| Stomach | 0.69 | 0.29 |
| Small intestine | 0.78 | 0.54 |
| Large intestine | 0.48 | 0.15 |
| Uterus | 0.52 | 0.24 |
| Skin | 0.51 | 0.13 |
| Muscle | 0.31 | 0.06 |
| Bone | 0.67 | 0.14 |
| Tumor | 4.80 | 2.18 |
| Brain | 0.05 | 0.02 |

Data expressed as %IA/g and presented as mean ± SD (n = 3).

### Example 8: Theranostic Potential of Amino Acid Sequence Derivatives of A1 and A2

### Example 8a:

Sequence derivatives of anti-human FOLR1 VHHs A1 and A2 were designed and produced. Amino acid substitutions were introduced to avoid post-translational modifications and issues towards targeting and biodistribution. One- or two-amino acid substitution derivatives from VHH A1 were A1-D59E, A1-N64E, A1-N64Q and A1-N64S (SEQ ID NO: 70, 71, 72 and 73, resp.), and derivatives of VHH A2 were A2-E90Q, A2-K29Q-E90Q, A2-K29R-E90Q, A2-M28A-E90Q and A2-M28T-E90Q (SEQ ID NO: 74, 75, 76, 77 and 78, resp.). VHHs A1-D59E, A1-N64E, A1-N64Q and A1-N64S each have 99% sequence identity to A1. VHH A2-E90Q has 99% sequence identity to A2. VHHs A2-K29Q-E90Q, A2-K29R-E90Q, A2-M28A-E90Q and A2-M28T-E90Q each have 98% sequence identity to A2. These derivatives were compared to the respective parental VHHs with regards to in vitro human FOLR1-binding parameters.

The antigen-binding kinetic parameters of untagged, purified VHH A1 and A2 variants were determined by surface plasmon resonance on immobilized human FOLR1 recombinant protein with a Biacore T200 instrument (GE Healthcare). The human FOLR1 recombinant protein (Sino Biological) was immobilized in 10 mM sodium acetate, pH 5.5 on a CM5 chip (GE Healthcare) via amine coupling chemistry to 250 RU. VHHs were analyzed using single cycle kinetics with a threefold serial dilution in HBS (5 nM-0.062 nM) and an analyte flow rate of 40 µl/min. The association phase took 5× 450 s and the dissociation phase 900 s. Regeneration was done by a single injection of 50 mM glycine-HCl, pH 2.5, 2 M MgCl₂ for 30 s at 30 µl/min, followed by 120 s stabilization. Binding curves were fitted using a ‘1:1 (antigen:analyte)’ binding model in Biacore T200 evaluation software.

Results are described in Table 26 and give an overview of the kinetic parameters of antigen binding, as determined by surface plasmon resonance with immobilized human FOLR1 recombinant protein. Untagged derivatives of VHH’s A1 (A1-D59E, A1-N64E, A1-N64Q and A1-N64S; SEQ ID NO: 70, 71, 72 and 73, resp.) and A2 (A2-E90Q, A2-K29Q-E90Q, A2-K29R-E90Q, A2-M28A-E90Q and A2-M28TE90Q; SEQ ID NO: 74, 75, 76, 77 and 78, resp.) showed similar KD values (affinities) and kd values (off-rates) as the parental VHH’s. These data also indicate that the presence of a His₆-tag on the VHH carboxyterminus does not affect the binding parameters of the VHH’s, as the values in table 26 (untagged VHHs) are similar to those in table 9 (His₆-tagged VHHs).

TABLE 26

Kinetic binding parameters ka, kd and KD of purified, untagged VHH A1 and A2 derivatives

| VHH | ka (M-1 s-1) | kd (s-1) | KD (M) | Rmax (RU) | Chi2 (RU2) |
|---|---|---|---|---|---|
| A1 | | | | | |
| A1-D59E | 2.3E+06 | 1.9E-04 | 8.3E-11 | 75.4 | 0.04 |
| A1-N64E | 1.8E+06 | 1.7E-04 | 9.2E-11 | 73.3 | 0.04 |
| A1-N64Q | 2.3E+06 | 8.2E-05 | 3.6E-11 | 74.0 | 0.07 |
| A1-N64S | 2.5E+06 | 1.4E-04 | 5.7E-11 | 73.2 | 0.09 |
| A2 | | | | | |
| A2-E90Q | 2.3E+06 | 6.0E-04 | 2.5E-10 | 67.2 | 0.15 |
| A2-K29Q-E90Q | 1.8E+06 | 4.8E-04 | 2.7E-10 | 68.9 | 0.08 |
| A2-K29R-E90Q | 1.9E+06 | 5.3E-04 | 2.9E-10 | 71.8 | 0.14 |
| A2-M28A-E90Q | 2.0E+06 | 1.6E-03 | 8.1E-10 | 68.6 | 0.16 |
| A2-M28T-E90Q | 2.3E+06 | 1.3E-03 | 5.6E-10 | 69.0 | 0.08 |

### Example 8b:

A1 and A2 parental untagged VHH’s and derivatives were radiolabeled with theranostic isotope ¹³¹I, as described in example 3, and subjected to saturation binding studies as described in example 6. All ¹³¹I-labeled A1 and A2 VHH-derivatives revealed a concentration-dependent relationship towards binding to human FOLR1-expressing cancer cells, with EC50 binding characteristics that are similar as the parental VHH (table 27).

TABLE 27

corresponding EC50 values for ¹³¹I-labeled, untagged A1 and A2 derivatives compared to parental VHHs, for binding to human FOLR1^{pos} SKOV-3 cells.

| VHH | EC50 ± SD (nM) |
|---|---|
| A1 | 1.1 ± 0.2 |
| A1-D59E | 2.1 ± 0.5 |
| A1-N64E | 2.5 ± 0.6 |
| A1-N64Q | 2.3 ± 0.4 |
| A1-N64S | 0.9 ± 0.3 |
| A2 | 2.4 ± 0.3 |
| A2-E90Q | 2.9 ± 0.9 |
| A2-K29Q-E90Q | 5.1 ± 0.6 |
| A2-K29R-E90Q | 3.6 ± 0.4 |
| A2-M28A-E90Q | 4.1 ± 0.3 |
| A2-M28T-E90Q | 4.8 ± 1.1 |

Data presented as mean ± SD (n = 3).

Collectively, these data indicate that one- or two aminoacid substitutions in A1 or A2 VHHs do not influence their targeting capacity to human FOLR1.

### Example 9: Synthesis of the Compound Comprising the VHH Linked to a Medicament Acting in the Brain and Therapeutic Use Thereof

In this example we describe the capacity of human FOLR1-targeting VHHs to transport a payload specifically to the blood cerebrospinal barrier (BCB). Indeed, ^{99m}Tc-labeled A1 (SEQ ID NO 53) shows increased uptake in brain in homozygous human FOLR1 knock-in mice, and more specifically in the brain choroid plexus.

To this, His₆-tagged human FOLR1-targeting A1 and control non-targeting VHH R3B23 were radiolabelled with the diagnostic radioisotope ^{99m}Tc, according to the radiochemical procedure described in example 3. After quality control (> 95%), a group of homozygous human FOLR1 knock-in mice (groups of 6 weeks and 6 months of age) were intravenously injected in the tail vein with about 1.3 mCi (± 5 µg) of ^{99m}Tc-labelled A1. Next, SPECT/CT was performed 1 h after tracer administration using a Vector⁺/CT MILabs system. SPECT/CT imaging was performed using a SPECT collimator and a spiral scan mode of six bed positions (2min30s per position). For CT, a normal scan mode of only one position was used. The obtained SPECT data are reconstructed with a 0.4 voxel size, 2 subsets and 4 iterations, after which images are fused and corrected for attenuation based on the CT scan. Images are analyzed using a medical image data analysis tool (AMIDE). Uptake values in a selection of organs and tissues were analyzed and expressed as % injected activity per cm³ (%IA/cc).

Next, mice were euthanized by cervical dislocation after 1.5 h, dissected after which different organs and tissues were collected. Organs and tissues of interest were weighed and measured for radioactivity using an automatic gamma counter, along with injection standards. Results were expressed as % of injected activity (IA)/g tissue.

Image quantification (table 15) and results obtained via dissections (table 16) indicated that the administration of ^{99m}Tc-labelled A1 led to focalised uptake in brain in homozygous human FOLR1 knock-in mice, which was specific for this human FOLR1-targeting VHH, as this elevated uptake was found absent for ^{99m}Tc-labelled non-targeting control VHH R3B23 administered to the same mice.

In an additional experiment, uptake in brain was significantly higher (p = 0.001 or lower, unpaired t-test) for ^{99m}Tc-labelled A1, both in mice of 6 week- and 6 month-old compared to ^{99m}Tc-labelled R3B23, as indicated from image quantification (Table 28) as well as the necropsy study (Table 29). No significant difference (p=0.905) was observed in brain uptake for ^{99m}Tc-labelled A1 in mice of 6 weeks and 6 months of age, indicating that human FOLR1-expression on the blood-cerebrospinal-fluid barrier (BCSFB) is not transient with age. These results indicate that human FOLR1-targeting VHHs are able to transport a payload specifically to the BCSFB.

TABLE 28

Uptake in different organs and tissues obtained from SPECT/CT images 1h after i.v. injection of ^{99m}Tc-labeled, His₆-tagged A1 and control non-targeted control VHH R3B23, in homozygous human FOLR1 knock-in mice at 6 weeks or 6 months of age

| %IA/cc | ^{99m}Tc-A1 (6 weeks) | | ^{99m}Tc-A1 (6 months) | | ^{99m}Tc-R3B23 (6 weeks) | | ^{99m}Tc-R3B23 (6 months) | |
|---|---|---|---|---|---|---|---|---|
| Organ/tissue | Mean | SD | Mean | SD | Mean | SD | Mean | SD |
| Kidney | 110.69 | 5.19 | 101.46 | 12.01 | 66.13 | 7.12 | 82.66 | 3.95 |
| Liver | 1.07 | 0.05 | 1.43 | 0.35 | 0.40 | 0.07 | 0.77 | 0.42 |
| Heart | 0.42 | 0.03 | 0.63 | 0.03 | 0.35 | 0.05 | 0.49 | 0.03 |
| Lung | 0.42 | 0.09 | 0.51 | 0.06 | 0.21 | 0.01 | 0.36 | 0.02 |
| Muscle | 0.17 | 0.08 | 0.16 | 0.01 | 0.13 | 0.02 | 0.16 | 0.15 |
| Bone | 0.19 | 0.03 | 0.24 | 0.04 | 0.23 | 0.13 | 0.11 | 0.02 |
| Brain | 0.63 | 0.07 | 0.63 | 0.02 | 0.07 | 0.01 | 0.08 | 0.004 |

Data expressed as %IA/cc and presented as mean ± SD (n = 3)

TABLE 29

Uptake in different organs and tissues obtained from dissections 1.5h after injection of ^{99m}Tc-labeled His₆-tagged A1 or nontargeting control VHH R3B23, in homozygous human FOLR1 knock-in mice at 6 weeks or 6 months of age

| %IA/g | ^{99m}Tc-A1 (6 weeks) | | ^{99m}Tc-A1 (6 months) | | ^{99m}Tc-R3B23 (6 weeks) | | ^{99m}Tc-R3B23 (6 months) | |
|---|---|---|---|---|---|---|---|---|
| Organ/tissue | Mean | SD | Mean | SD | Mean | SD | Mean | SD |
| Blood | 0.66 | 0.05 | 0.95 | 0.11 | 0.83 | 0.05 | 0.84 | 0.08 |
| Lymphnodes | 1.03 | 0.21 | 0.86 | 0.37 | 0.43 | 0.23 | 0.59 | 0.38 |
| Thymus | 0.28 | 0.03 | 0.45 | 0.13 | 0.11 | 0.02 | 0.25 | 0.03 |
| Heart | 0.47 | 0.03 | 0.57 | 0.04 | 0.28 | 0.04 | 0.38 | 0.02 |
| Lung | 1.29 | 0.23 | 1.18 | 0.04 | 0.68 | 0.17 | 0.79 | 0.04 |
| Liver | 1.98 | 0.13 | 2.36 | 0.54 | 0.78 | 0.01 | 0.82 | 0.09 |
| Pancreas | 0.70 | 0.08 | 0.96 | 0.04 | 0.18 | 0.04 | 0.16 | 0.02 |
| Spleen | 0.69 | 0.03 | 0.90 | 0.13 | 0.25 | 0.01 | 0.30 | 0.04 |
| Adrenals | 1.47 | 0.34 | 1.30 | 0.03 | 0.42 | 0.04 | 0.69 | 0.31 |
| Kidneys | 290.99 | 39.40 | 254.50 | 40.69 | 223.47 | 19.92 | 194.62 | 18.67 |
| Stomach | 0.65 | 0.21 | 0.69 | 0.02 | 0.53 | 0.09 | 0.67 | 0.19 |
| Small intestine | 0.57 | 0.09 | 0.77 | 0.13 | 0.28 | 0.04 | 0.42 | 0.11 |
| Large intestine | 0.88 | 0.10 | 0.95 | 0.33 | 0.35 | 0.01 | 0.36 | 0.04 |
| Ovary | 13.05 | 2.18 | 3.99 | 1.57 | 0.62 | 0.13 | 0.74 | 0.70 |
| Uterus | 1.25 | 0.05 | 1.39 | 0.04 | 0.65 | 0.14 | 0.68 | 0.14 |
| Skin | 0.95 | 0.22 | 0.72 | 0.40 | 0.74 | 0.34 | 0.44 | 0.08 |
| Muscle | 0.25 | 0.07 | 0.28 | 0.00 | 0.17 | 0.04 | 0.17 | 0.03 |
| Bone | 0.53 | 0.04 | 0.35 | 0.05 | 0.15 | 0.03 | 0.21 | 0.07 |
| Joint | 0.93 | 0.48 | 0.55 | 0.04 | 0.34 | 0.03 | 0.28 | 0.07 |
| Brain | 0.52 | 0.05 | 0.51 | 0.07 | 0.02 | 0.01 | 0.03 | 0.01 |

Data expressed as %IA/g and presented as mean ± SD (n = 3).

1-20. (canceled)

21. A compound comprising:
a heavy chain antibody (VHH) or a fragment thereof that specifically binds an epitope of human folate receptor alpha (FOLR1), but does not bind human folate receptor beta (FOLR2) or human folate receptor gamma (FOLR3), said VHH being linked to a radionuclide; and
wherein said VHH comprises an amino acid sequence having at least 63% sequence identity with at least one of SEQ ID NO:8 or 9, over the full length of said sequence, or over at least 50% of the length of said sequence.

22. The compound according to claim 21, said VHH comprising the amino acid sequence of SEQ ID NO:8, 9, 12, or 19.

23. The compound according to claim 21, wherein said VHH or fragment thereof specifically binds to human FOLR1 with a K_{D} from 10⁻⁹ to 10⁻¹¹ moles/liter, and/or a k_{off} from 10⁻⁴ to 10⁻² s⁻¹.

24. The compound according to claim 23, wherein the K_{D} and/or k_{off} is assessed using surface plasmon resonance (SPR).

25. The compound according to claim 24, wherein said SPR utilizes human FOLR1 recombinant protein as an immobilized ligand.

26. The compound according to claim 21, wherein said radionuclide is chosen from the group consisting of α-emitting radionuclides and β-emitting radionuclides.

27. The compound according to claim 26, wherein said radionuclide is chosen from the group consisting of: actinium-225, astatine-211, bismuth-212, bismuth-213, caesium-137, chromium-51, cobalt-60, copper-67, dysprosium-165, erbium-169, fermium-255, gold-198, holium-166, iodine-125, iodine-131, iridium-192, iron-59, lead-212, lutetium-177, molydenum-99, palladium-103, phosphorus-32, potassium-42, rhenium-186, rhenium-188, samarium-153, radium-223, radium-224, ruthenium-106, sodium-24, strontium-89, scandium-47, terbium-149, terbium-161, terbium-149, thorium-227, xenon-133, ytterbium-169, ytterbium-177, and yttrium-90.

28. The compound according to claim 26, wherein said radionuclide is chosen from the group consisting of positron-emitting radioisotopes (PET) and γ-emitting radioisotopes (SPECT).

29. The compound according to claim 28, wherein said radionuclide is chosen from the group consisting of: iodine-131, yttrium-90, iodine-125, lutetium-177, rhenium-186, rhenium-188, terbium-161, technetium-99m, indium-111, xenon-133, thallium-201, fluorine-18, gallium-68, gallium-67, copper-67, scandium-44, scandium-43, iodine-123, iodine-124, zirconium-89, and copper-64.

30. The compound according to claim 29, wherein said radionuclide is iodine-131.

31. The compound according to claim 30, wherein said VHH or fragment thereof and said radionuclide are separated by a linker.

32. The compound according to claim 31, wherein said linker is a benzoate linker.

33. The compound according to claim 32, wherein said linker comprises N-succinimidyl-4-guanidinomethy1-3-[1-131]iodobenzoate or a derivative thereof.

34. A composition comprising a compound according to claim 21 and a pharmaceutically acceptable excipient.

35. A method for treating a cancer associated with an expression of human FOLR1 on a cancer cell, comprising administering the compound according to claim 21.

36. The method according to claim 35, further comprising administering an additional antibody or antibody fragment.

37. A method for treating a cancer associated with an expression of human FOLR1 on a cancer cell in a subject, comprising administering the composition according to claim 34 to the subject.

38. The method according to claim 37, further comprising administering an additional antibody or antibody fragment.

39. A diagnostic composition comprising the compound according to claim 21.

40. A method for assessing expression of human FOLR1 in a subject, comprising administering the diagnostic composition according to claim 39 to the subject.


SEQUENCE LISTING (86 sequences)

SEQ ID NO: 1 - protein, 257 aa, Homo sapiens
  REGION 1..257: note = Human FOLR1
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 2 - protein, 78 aa, synthetic construct
NHMSWYRQAPGKQRELAAIITSDGNTNYPDSVKGRFTISRDNAKNTVYLQMNSLKPEDTAVYYCNTFGRSVAGFYDYW

SEQ ID NO: 3 - protein, 76 aa, synthetic construct
DLAWYRRAPGKQRELAAIITSGGSTNYPDSVKGRFTISRDNAKNTVYLEMNSLKPEDTAVYYCNALRPWPQFSDYW

SEQ ID NO: 4 - protein, 30 aa, synthetic construct
NHMSWYRQAPGKQRELAAIITSDGNTNYPD

SEQ ID NO: 5 - protein, 14 aa, synthetic construct
NTFGRSVAGFYDYW

SEQ ID NO: 6 - protein, 29 aa, synthetic construct
DLAWYRRAPGKQRELAAIITSGGSTNYPD

SEQ ID NO: 7 - protein, 13 aa, synthetic construct
NALRPWPQFSDYW

SEQ ID NO: 8 - protein, 119 aa, synthetic construct
DVQLVESGGGLVQPGGSLRLSCAASESIFSRNHMSWYRQAPGKQRELAAIITSDGNTNYPDSVKGRFTISRDNAKNTVYLQMNSLKPEDTAVYYCNTFGRSVAGFYDYWGQGTQVTVSS

SEQ ID NO: 9 - protein, 118 aa, synthetic construct
DVQLVESGGGLVQAGGSLRLSCAADEMKFSNYDLAWYRRAPGKQRELAAIITSGGSTNYPDSVKGRFTISRDNAKNTVYLEMNSLKPEDTAVYYCNALRPWPQFSDYWGQGTQVTVSS

SEQ ID NO: 10 - protein, 8 aa, synthetic construct
ESIFSRNH

SEQ ID NO: 11 - protein, 7 aa, synthetic construct
ITSDGNT

SEQ ID NO: 12 - protein, 13 aa, synthetic construct
NTFGRSVAGFYDY

SEQ ID NO: 13 - protein, 25 aa, synthetic construct
DVQLVESGGGLVQPGGSLRLSCAAS

SEQ ID NO: 14 - protein, 17 aa, synthetic construct
MSWYRQAPGKQRELAAI

SEQ ID NO: 15 - protein, 38 aa, synthetic construct
NYPDSVKGRFTISRDNAKNTVYLQMNSLKPEDTAVYYC

SEQ ID NO: 16 - protein, 11 aa, synthetic construct
WGQGTQVTVSS

SEQ ID NO: 17 - protein, 8 aa, synthetic construct
EMKFSNYD

SEQ ID NO: 18 - protein, 7 aa, synthetic construct
ITSGGST

SEQ ID NO: 19 - protein, 12 aa, synthetic construct
NALRPWPQFSDY

SEQ ID NO: 20 - protein, 25 aa, synthetic construct
DVQLVESGGGLVQAGGSLRLSCAAD

SEQ ID NO: 21 - protein, 17 aa, synthetic construct
LAWYRRAPGKQRELAAI

SEQ ID NO: 22 - protein, 38 aa, synthetic construct
NYPDSVKGRFTISRDNAKNTVYLEMNSLKPEDTAVYYC

SEQ ID NO: 23 - protein, 11 aa, synthetic construct
WGQGTQVTVSS

SEQ ID NO: 24 - protein, 121 aa, synthetic construct
DVQLVESGGGLVQAGGSLRLSCGVSGNMFSMNLMGWYRQPPGKERDWVAVITKDGSTNYADSVKGRFTISRDNAKNTVSLQMNSLKPEDTAVYYCNVQDGSGWIRAWGWDWGQGTQVTVSS

SEQ ID NO: 25 - protein, 8 aa, synthetic construct
GNMFSMNL

SEQ ID NO: 26 - protein, 7 aa, synthetic construct
ITKDGST

SEQ ID NO: 27 - protein, 15 aa, synthetic construct
NVQDGSGWIRAWGWD

SEQ ID NO: 28 - protein, 25 aa, synthetic construct
DVQLVESGGGLVQAGGSLRLSCGVS

SEQ ID NO: 29 - protein, 17 aa, synthetic construct
MGWYRQPPGKERDWVAV

SEQ ID NO: 30 - protein, 38 aa, synthetic construct
NYADSVKGRFTISRDNAKNTVSLQMNSLKPEDTAVYYC

SEQ ID NO: 31 - protein, 11 aa, synthetic construct
WGQGTQVTVSS

SEQ ID NO: 32 - protein, 119 aa, synthetic construct
DVQLVESGGGLARAGGSLRLSCAASGRAFSSTYAMGWFRQAPGKEREFVADINWSEGSPMYIDSVKDRFTISRDNARNSLYLQMNSLKSEDTAVYYCAARRFGQGYEYWGQGTQVTVSS

SEQ ID NO: 33 - protein, 9 aa, synthetic construct
GRAFSSTYA

SEQ ID NO: 34 - protein, 8 aa, synthetic construct
INWSEGSP

SEQ ID NO: 35 - protein, 11 aa, synthetic construct
AARRFGQGYEY

SEQ ID NO: 36 - protein, 25 aa, synthetic construct
DVQLVESGGGLARAGGSLRLSCAAS

SEQ ID NO: 37 - protein, 17 aa, synthetic construct
MGWFRQAPGKEREFVAD

SEQ ID NO: 38 - protein, 38 aa, synthetic construct
MYIDSVKDRFTISRDNARNSLYLQMNSLKSEDTAVYYC

SEQ ID NO: 39 - protein, 11 aa, synthetic construct
WGQGTQVTVSS

SEQ ID NO: 40 - protein, 121 aa, synthetic construct
DVQLVESGGGLVQAGGSLRLSCGVSGNIFSMNLMGWYRQPPGKEREWVALITKDGSTNYEDSVKGRFTISRDNAKNTVSLQMNSLKPEDTAVYYCNAQDGSGWIRAWGWDWGQGTQVTVSS

SEQ ID NO: 41 - protein, 8 aa, synthetic construct
GNIFSMNL

SEQ ID NO: 42 - protein, 7 aa, synthetic construct
ITKDGST

SEQ ID NO: 43 - protein, 15 aa, synthetic construct
NAQDGSGWIRAWGWD

SEQ ID NO: 44 - protein, 25 aa, synthetic construct
DVQLVESGGGLVQAGGSLRLSCGVS

SEQ ID NO: 45 - protein, 17 aa, synthetic construct
MGWYRQPPGKEREWVAL

SEQ ID NO: 46 - protein, 38 aa, synthetic construct
NYEDSVKGRFTISRDNAKNTVSLQMNSLKPEDTAVYYC

SEQ ID NO: 47 - protein, 11 aa, synthetic construct
WGQGTQVTVSS

SEQ ID NO: 48 - protein, 134 aa, synthetic construct
QVQLQESGGGLVQPGGSLRLSCAASESIFSRNHMSWYRQAPGKQRELAAIITSDGNTNYPDSVKGRFTISRDNAKNTVYLQMNSLKPEDTAVYYCNTFGRSVAGFYDYWGQGTQVTVSSGRYPYDVPDYGSGRA

SEQ ID NO: 49 - protein, 133 aa, synthetic construct
QVQLQESGGGLVQAGGSLRLSCAADEMKFSNYDLAWYRRAPGKQRELAAIITSGGSTNYPDSVKGRFTISRDNAKNTVYLEMNSLKPEDTAVYYCNALRPWPQFSDYWGQGTQVTVSSGRYPYDVPDYGSGRA

SEQ ID NO: 50 - protein, 136 aa, synthetic construct
QVQLQESGGGLVQAGGSLRLSCGVSGNMFSMNLMGWYRQPPGKERDWVAVITKDGSTNYADSVKGRFTISRDNAKNTVSLQMNSLKPEDTAVYYCNVQDGSGWIRAWGWDWGQGTQVTVSSGRYPYDVPDYGSGRA

SEQ ID NO: 51 - protein, 134 aa, synthetic construct
QVQLQESGGGLARAGGSLRLSCAASGRAFSSTYAMGWFRQAPGKEREFVADINWSEGSPMYIDSVKDRFTISRDNARNSLYLQMNSLKSEDTAVYYCAARRFGQGYEYWGQGTQVTVSSGRYPYDVPDYGSGRA

SEQ ID NO: 52 - protein, 136 aa, synthetic construct
QVQLQESGGGLVQAGGSLRLSCGVSGNIFSMNLMGWYRQPPGKEREWVALITKDGSTNYEDSVKGRFTISRDNAKNTVSLQMNSLKPEDTAVYYCNAQDGSGWIRAWGWDWGQGTQVTVSSGRYPYDVPDYGSGRA

SEQ ID NO: 53 - protein, 125 aa, synthetic construct
DVQLVESGGGLVQPGGSLRLSCAASESIFSRNHMSWYRQAPGKQRELAAIITSDGNTNYPDSVKGRFTISRDNAKNTVYLQMNSLKPEDTAVYYCNTFGRSVAGFYDYWGQGTQVTVSSHHHHHH

SEQ ID NO: 54 - protein, 124 aa, synthetic construct
DVQLVESGGGLVQAGGSLRLSCAADEMKFSNYDLAWYRRAPGKQRELAAIITSGGSTNYPDSVKGRFTISRDNAKNTVYLEMNSLKPEDTAVYYCNALRPWPQFSDYWGQGTQVTVSSHHHHHH

SEQ ID NO: 55 - protein, 127 aa, synthetic construct
DVQLVESGGGLVQAGGSLRLSCGVSGNMFSMNLMGWYRQPPGKERDWVAVITKDGSTNYADSVKGRFTISRDNAKNTVSLQMNSLKPEDTAVYYCNVQDGSGWIRAWGWDWGQGTQVTVSSHHHHHH

SEQ ID NO: 56 - protein, 125 aa, synthetic construct
DVQLVESGGGLARAGGSLRLSCAASGRAFSSTYAMGWFRQAPGKEREFVADINWSEGSPMYIDSVKDRFTISRDNARNSLYLQMNSLKSEDTAVYYCAARRFGQGYEYWGQGTQVTVSSHHHHHH

SEQ ID NO: 57 - protein, 127 aa, synthetic construct
DVQLVESGGGLVQAGGSLRLSCGVSGNIFSMNLMGWYRQPPGKEREWVALITKDGSTNYEDSVKGRFTISRDNAKNTVSLQMNSLKPEDTAVYYCNAQDGSGWIRAWGWDWGQGTQVTVSSHHHHHH

SEQ ID NO: 58 - DNA, 774 bp, Homo sapiens
  source 1..774: mol_type = genomic DNA
atggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagctga

SEQ ID NO: 59 - DNA, 15927 bp, synthetic construct
  source 1..15927: mol_type = other DNA
cgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcccgggctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctaggatctgcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagagatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcaggatccatggcacgcgcttctacaaggcgctggccgaagaggtgcgggagtttcacgccaccaagatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgctcgacattgggtggaaacattccaggcctgggtggagaggctttttgcttcctcttgcaaaaccacactgctcgacattgggtggaaacattccaggcctgggtggagaggctttttgcttcctcttgaaaaccacactgctcgatttgttagcagcctcgaatcaacccgggcgatcctaggcgatgagatctagctgtcgcgaagagtggcgcgccagcctgggcctacaaacaaacaaacaaacaaattctacaaatcttcaatttatccgtcgcttcttaaggtttttgttgtttgttttgttttattctgaaacagggtcccactgtgtttgctgtggttgccctggctgtctttgaacttagagatctgcctctctctgctcccctagtgccgggatcaaaggcctgtatctccaaactaatacttaatctttaaattcttttttttttttttttttttttcgagacagggttcctctgtatagccctggctgtcctggaactcactttgtagaccagactggcctcgaactcagaaatttgtctgcctctgcctcccgagtgctgggattaaaggtgtgtgccaacacgcccggctcatctttaaatttttttatacaaagtcaattggtatcaaaatctgatttgtcaatcgtggtggtacaaggcagatctcttttgagttcaaggccagcctgatctgcaagaccagcttgatctataagaccaacctggtctatagcaagtttcagagtagccagggctacatagtgagaccttacctaaagatccaaaccaaataaaacagaattcctgagttaattactatacaggcgctagagagatggttcaggcggtaaaggcacttgctgacaacctgagaacaatccctgggtcccttacgctagaaggagagaaccaactcctgcaagttgtccttccacctctacatacaggttcaggcatgcacacgtgtatacccaaatgaattaaatacatagtagatactaggcaaaggtttttgttgttgttgttgtttggttggttttgtttttttcgagacagggtttctctgtgtagccctggctgtcctggaactcattctgtagaccaggctggccttgaactcaggcctgcctctgcctcccaagtgctgggattaaaggcgtgagccaccactgcctggcagccaagctctttttacagaagttgaaaacttgtgacttgttatgaacaatagtcctcagcattccaagaagttatgtgaccttactcaagttgggcttacaaattagaagcattttttgttttatagatctttttagcatagtaatttcaatttaaaacatcacaaattgaggactggagagacagttcatgggtgaagagcactggctatttttagaggccccacattcgtttcccagcacccacatggttgctcacagccatctctaaccctagtcctaagggccctgacaccctcttctggtctctgggtacactgcatgcatgtggtgtatagaatacatgcagagtggatcagtgtgacggacgaatggttttagtgactagttgtgtctagatcctattgcactgatggtcatgaagttgaaacatgggggaaaatgaactttatacccttcttcatgacttctgtccttttgcctgcctcctttctcatctcctaatattacagtcttggtttcctctctaaatttttagacttttaaccctcacctaaacctgtatcagcttttataaaaatcttttcaaaacttcacactgaagcatctgcctccaaaggttttgaatgtgaacgtgggtaaactctgttctttcaaatggcccatctcttattttttaattgccctgtgtgagtctcaggaccactaagtctaacaggctgtgaccagtgattgtctctagggcatctgagcctcacagagtctgggaagactgacaggaggaggtgacccaaggtctgtgagtgcaggctccacccactggagctgagcacacacttggaggttccacttaccttagctctgccttcagggtctgacatggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagctgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcaagcttctcgagggacctaataacttcgtatagcatacattatacgaagttatattaagggttccgcaagctctagtcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagagaattgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatggtttagttcctcaccttgtcgtattatactatgccgatatactatgccgatgattaattgtcaacaggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaatttcgacgacctgcagccaagctagcttcgcgagctcgaccgaacaaacgacccaacacccgtgcgttttattctgtctttttattgccgctcagctttacagtgacaatgacggctggcgactgaatattagtgcttacagacagcactacatattttccgtcgatgttgaaatcctttctcatatgtcaccataaatatcaaataattatagcaatcatttacgcgttaatggctaatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacaccttcctcttcttcttgggcatggccgcaggaaagcagagccctgaagctcccatcaccggccaataagagccaagcctgcagtgtgacctcatagagcaatgtgccagccagcctgaccccaagggccctcaggcttgggcacactgtctctaggaccctgagagaaagacatacccatttctgcttagggccctgaggatgagcccaggggtggcttggcactgaagcaaaggacactggggctcagctggcagcaaagtgaccaggatgctgaggctttgacccagaagccagaggccagaggccaggacttctcttggtcccagtccaccctcactcagagctttaccaatgccctctggatagttgtcgggtaacggtggacgccactgattctctggccagcctaggacttcgccattccgctgattctgctcttccagccactggctgaccggttggaagtactccagcagtgccttggcatccagggcatctgagcctaccaggtccttcagtacctcctgccagggcctggagcagccagcctgcaacacctgcctgccaagcagagtgaccactgtgggcacaggggacacagggtggggcccacaacagcaccattgtccacttgtccctcactagtaaaagaactctagggttgcggggggtgggggaggtctctgtgaggctggtaagggatatttgcctggcccatggagctagcttggctggacgtaaactcctcttcagacctaataacttcgtatagcatacattatacgaagttatattaagggttattgaatatgatcggaattgggctgcaggaattcgatagcttggctgcaggtcgaccctaggtgtatgctgcccagtcacttttctgccagcctttgaagacttgagaaggagactctcatatctgagaagcctttggaggcaggcatctagcgaacacttggactgtggtcctcagcttgagggctggagggcttgagggctctgtgttataacagttgtttgccatagtgcttttagtatcccaaagctcactaaacatttaataaaatcagtgtgatgcaacaactatgaagtcaaccagcagcaggtctgctattggggaggtacaatcagtgcagacaacaaagtgggaggggggtctcaaaaaaagccaagatgagggctggagagttggctcagtggttaaaagcacttgttgagcttgcagaataccaaggtctgatccacaacatccaaggtggtggatccatcccctttcaggggatgcaatgccctcttctgacctctgcaggcaccaggtgtgcatgtgatacatataaaataaataaagctaataagtaaataagcaaataggcctggggatgtagctcagctgatagagtacctgcatggcagtcacaaatctctagcaccatatggaactggatgtgttgacacataaatgtaacctcagcccaggggaggtagaggtagtcagggtcagaagttcaaggttaccttcagctacatagtgagttcagtgccagtctggaatatatgaaaccctgtcaagaaagggatagagagagggaggagagagggaggaggctatttttattatttattttttgagacagggttctactatgtaattctggctagcctgactctcacagagatctgtctgcctcaatctcctgagtgctaggattaaaggtggacataccacaccacaaaaaaatttaaaaaagcaaaagaaaagcaatctgtatcatgtgcatcaatggataaatgagcatgttgactctattagctgagagctgtcaggttctatttgctgctggttttttgtttatttgtttttatgtatttatttttatggtattttgcatgcatatttgtcaatcaaagaagtgcagtgcatgcaaaggccagaagagggtgttggagtctccacgaccagagttataggtggttgtgagcctccacgtgcatgctgggagctgaatccaagtcctctgtaagaatgacaagtgctctcaacccctgagccatcactccaggctctatttactttttgtaatacttaagctaagcagtggtgatgcatgtgtttaatcctagcacttggaggcagaggcagacggatttctgagtttgagatcagcctggtctacatagtgaggtccaggatagtcagatctacacagagaaaccatgttctaaataaataaataaataaataagcctaaataaaatttaaaaatatttaagatttgtaccttgagaattccatatgtacatacaatgtatttccatcatgtgtagctcaggcacacccaccctttgggttctactttgtcttttcctcccacaggagcatggtcagcctatttgaaccacatttgactcttccctgggaatcacaaactatcaatagatcctcagctagggatgaggttcctgtgtccttctctaacccactgctgctgctaatttgttcgtttgtttgtgtctgggagacagggtctctcttgtagcctgtctggcctggaactcaaacaggctggcctcccgggataaaggattaaaggtgtgtgcctccatacctgtgctgctgttttgaaacactgagcacaaggttgctagttttagattgagcagtggctaccttgagcatccttaatttacaaacttattcaaaccttccttccttccttccttccttccttccttccttccttccttccttccttcttttctctctttttttaaaatttatttttgtcacttgggagtcagaggcaggagaatccactgagccacagagctagcctggtctacatagcaagttccaagtaagccagagctatatagtgagaccctgtatcaaaaataaatagccgggcaatggtgatgcacgcctttaatccaagcacttgggaggcagaggcaggcgaatttctgagttcgagaccagcctagtctacagaatgagttccaggacagccaggactatacagagaaaccctgtctcgaaaaaccaaaccaaacaaaaaaaaaaaaaaaaaggaaagaaagaaaaattatatatatgcttttgtttagtgtaggtgtgtgtgtctgtgtgaatgaataccacacatgtatgtatgagttctggtggaactaaaggaaagcatcagatcctttggagctggagctgcagccatatttgagccaggacataggtgctgggcagtagactattaaatgctgagtcatctctctagattcttagggtgtttttgtttttgttttttaaactgggcatggtgccacatgccctttatcctagcactctgagaagcagaggaaaacagatctctgtgagttagaggccagtatgatccacatagcaaatttcagggttatcccagtctcaaaaacaaacaaacaaacaaacaaacaaacaaacaaacagaataaaacaacaacaaaaaacttgttttgaaggtgtgttttgaaactttgttatgtgctaagttccaggccaagagtcgattttcaggagtaagttagcgtattgcgagtgttctttttctcggtgacatttaatgcgaccatctggtggcactgcaagtccacatgcctttctcttgcttttatttatttatttatttatttatttatttccccaaagcgattggcatgttgggtttatcattttatcccttatttcctaggaaagtggcttggggtgggtcggggtctcataaagactagaatcaaggggctggagagatggctcagcagttaagagcacctactgctcttctgaaggtcatgagttcaaatcctagcaaccacatgatggcttacaaccatccgtaatgagatctgatgccctcttctagggtgtctgaagacagctactgtgtacttacatataataaataaataaatcttaaaaaaaaaacaaaacaaaactagaattaaagaactagatgtatgatcagctgtggtggtggtaagcctacctctggggatgctaggttaacctgatctatatagtgagttacaggccagccagggttacctagtgaaaacctggctttttaaaattttaattaactaaggaaagttaaacctggatgtggtagcacgcatctcaccacttgagaagcagaagagattcaggagtccaaggctatccttggttacatatagagtctgaagccacttagactctatgaggctgctcagaaaaaagggaccgggaagacaacacttgcctcagaaccataaggacccgagtttaatccttaggactcatgtacaaatgcctagggtaatgtgggtttatgacctcagcaccgaggagataagatcagtgagtccgaatgtttatcaaaggttagaatagcttcatttgtggtccccaggccagtgagagaccctgcctcaaagtgggcagactgcctgaggttgtcttctgacctctacacttgcaaacaaatgtgtgtgtgaacactcatagatacacaaataggcacacacacacacaaagcacatgtgcaaagaaaaaaataccaaggcatagtgagtggcccatgcctttaattctagcacttagaaggcagaggcagtcagatagcttggagtttaattcaagtctggtttacatagtgaattacagacagttgtgagcctccattgtgggtgcttgaaaccaaaccctggtcttctgcaagagcaaccagtgtgctcttacccactgagccagatgtctcttctgggcttctacagccagcttaacagcaagtatcgaaatggctaggcaatcaaggactcttcaaagtgcaaggctgcatgtggaatattctgtctgtgcttggccacggtcctgtgatttccatctcacagtgcagcccctggaagacgaattcctgctgttccacgaacacaagccaggaagcacataaggacatttcctacctgtaccggttcaactggaaccactgcggaactatgacatcggaatgcaaacggcactttatccaagacacctgcctctatgagtgttccccgaacttgggaccctggatccagcaggtacgagtgtcccccagtgccagcggagtagctgtgctctccaaatctctacaggctatgtctgcagctccgattctactcaggctgcatttctgaggttcctgagttcaaaaccttttacaccctctctcaggtggaccagagctggcgcaaagagcggatccttgatgttcccctgtgcaaagaggactgtcagcagtggtgggaggactgccagagctcttttacctgcaagagcaattggcacaagggatggaactggtcctcgggtaaggagtaggtggagtgggcgcggccgcgtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaa

SEQ ID NO: 60 - protein, 134 aa, synthetic construct
QVQLQESGGGSVQAGGSLRLSCAASGDTGYMGWFRQAPGKEREGVAVINSDSGVGSTYYADSVKGRFTISRDNAKNTVYLQMNSLKPEDTAIYYCAAGHFSDYVSPWTWREIYRYNVWGQGTQVTVSSHHHHHH

SEQ ID NO: 61 - protein, 80 aa, synthetic construct
NLMGWYRQPPGKERDWVAVITKDGSTNYADSVKGRFTISRDNAKNTVSLQMNSLKPEDTAVYYCNVQDGSGWIRAWGWDW

SEQ ID NO: 62 - protein, 30 aa, synthetic construct
NLMGWYRQPPGKERDWVAVITKDGSTNYAD

SEQ ID NO: 63 - protein, 16 aa, synthetic construct
NVQDGSGWIRAWGWDW

SEQ ID NO: 64 - protein, 76 aa, synthetic construct
AMGWFRQAPGKEREFVADINWSEGSPMYIDSVKDRFTISRDNARNSLYLQMNSLKSEDTAVYYCAARRFGQGYEYW

SEQ ID NO: 65 - protein, 30 aa, synthetic construct
AMGWFRQAPGKEREFVADINWSEGSPMYID

SEQ ID NO: 66 - protein, 12 aa, synthetic construct
AARRFGQGYEYW

SEQ ID NO: 67 - protein, 80 aa, synthetic construct
NLMGWYRQPPGKEREWVALITKDGSTNYEDSVKGRFTISRDNAKNTVSLQMNSLKPEDTAVYYCNAQDGSGWIRAWGWDW

SEQ ID NO: 68 - protein, 30 aa, synthetic construct
NLMGWYRQPPGKEREWVALITKDGSTNYED

SEQ ID NO: 69 - protein, 16 aa, synthetic construct
NAQDGSGWIRAWGWDW

SEQ ID NO: 70 - protein, 119 aa, synthetic construct
DVQLVESGGGLVQPGGSLRLSCAASESIFSRNHMSWYRQAPGKQRELAAIITSEGNTNYPDSVKGRFTISRDNAKNTVYLQMNSLKPEDTAVYYCNTFGRSVAGFYDYWGQGTQVTVSS

SEQ ID NO: 71 - protein, 119 aa, synthetic construct
DVQLVESGGGLVQPGGSLRLSCAASESIFSRNHMSWYRQAPGKQRELAAIITSDGETNYPDSVKGRFTISRDNAKNTVYLQMNSLKPEDTAVYYCNTFGRSVAGFYDYWGQGTQVTVSS

SEQ ID NO: 72 - protein, 119 aa, synthetic construct
DVQLVESGGGLVQPGGSLRLSCAASESIFSRNHMSWYRQAPGKQRELAAIITSDGQTNYPDSVKGRFTISRDNAKNTVYLQMNSLKPEDTAVYYCNTFGRSVAGFYDYWGQGTQVTVSS

SEQ ID NO: 73 - protein, 119 aa, synthetic construct
DVQLVESGGGLVQPGGSLRLSCAASESIFSRNHMSWYRQAPGKQRELAAIITSDGSTNYPDSVKGRFTISRDNAKNTVYLQMNSLKPEDTAVYYCNTFGRSVAGFYDYWGQGTQVTVSS

SEQ ID NO: 74 - protein, 118 aa, synthetic construct
DVQLVESGGGLVQAGGSLRLSCAADEMKFSNYDLAWYRRAPGKQRELAAIITSGGSTNYPDSVKGRFTISRDNAKNTVYLQMNSLKPEDTAVYYCNALRPWPQFSDYWGQGTQVTVSS

SEQ ID NO: 75 - protein, 118 aa, synthetic construct
DVQLVESGGGLVQAGGSLRLSCAADEMQFSNYDLAWYRRAPGKQRELAAIITSGGSTNYPDSVKGRFTISRDNAKNTVYLQMNSLKPEDTAVYYCNALRPWPQFSDYWGQGTQVTVSS

SEQ ID NO: 76 - protein, 118 aa, synthetic construct
DVQLVESGGGLVQAGGSLRLSCAADEMRFSNYDLAWYRRAPGKQRELAAIITSGGSTNYPDSVKGRFTISRDNAKNTVYLQMNSLKPEDTAVYYCNALRPWPQFSDYWGQGTQVTVSS

SEQ ID NO: 77 - protein, 118 aa, synthetic construct
DVQLVESGGGLVQAGGSLRLSCAADEAKFSNYDLAWYRRAPGKQRELAAIITSGGSTNYPDSVKGRFTISRDNAKNTVYLQMNSLKPEDTAVYYCNALRPWPQFSDYWGQGTQVTVSS

SEQ ID NO: 78 - protein, 118 aa, synthetic construct
DVQLVESGGGLVQAGGSLRLSCAADETKFSNYDLAWYRRAPGKQRELAAIITSGGSTNYPDSVKGRFTISRDNAKNTVYLQMNSLKPEDTAVYYCNALRPWPQFSDYWGQGTQVTVSS

SEQ ID NO: 79 - protein, 9 aa, synthetic construct
YPYDVPDYA

SEQ ID NO: 80 - protein, 10 aa, synthetic construct
YPYDVPDYGS

SEQ ID NO: 81 - protein, 15 aa, synthetic construct
SPSTPPTPSPSTPPC

SEQ ID NO: 82 - DNA, 357 bp, synthetic construct
  source 1..357: mol_type = other DNA
gatgtgcagctggtggagtctgggggaggcttggtgcagcctggggggtctctgagactctcctgtgcagcctctgaaagcatcttcagtcgcaatcacatgagctggtaccgccaggctccagggaagcagcgcgagttggccgcgattattactagtgatggtaacacaaactatccagactccgtgaagggccgattcaccatctccagagacaacgctaagaacacggtgtatctgcaaatgaacagcctgaaacctgaggacacggccgtctattactgtaataccttcggtcgtagcgtagctggtttttatgactactggggccaggggacccaggtcaccgtctcctca

SEQ ID NO: 83 - DNA, 354 bp, synthetic construct
  source 1..354: mol_type = other DNA
gatgtgcagctggtggagtctgggggaggcttggtgcaggctggggggtctctgagactctcctgtgcagccgatgaaatgaaattcagtaattatgacttggcctggtaccgccgggctccagggaagcagcgcgagttggccgcaattattactagtggtggtagcacaaactatccggactccgtgaagggccgattcaccatctccagagacaacgccaagaacacggtgtatctagaaatgaacagcctgaaacctgaggacacagccgtctattactgcaatgcacttcgaccctggcctcaatttagcgactactggggccaggggacccaggtcaccgtctcctca

SEQ ID NO: 84 - DNA, 363 bp, synthetic construct
  source 1..363: mol_type = other DNA
gatgtgcagctggtggagtctgggggaggcttggtgcaggctggggggtctctgagactctcctgtggagtctctgggaacatgttcagtatgaatctcatgggctggtaccgccaacctccagggaaggagcgcgactgggtcgcagttattactaaagatgggagcacaaactatgcagactccgtgaagggccgattcaccatctccagggacaacgccaagaacacggtgtctctgcaaatgaacagcctgaaacctgaggacacagccgtctactactgtaatgtccaggatggtagtggttggattcgggcgtggggctgggactggggccagggaacccaggtcaccgtctcctca

SEQ ID NO: 85 - DNA, 357 bp, synthetic construct
  source 1..357: mol_type = other DNA
gatgtgcagctggtggagtctggaggaggattggccagggctgggggctctctgagactctcctgtgcagcctctggacgcgccttcagtagtacgtatgccatgggttggttccgccaggctccagggaaggagcgtgagtttgtagcggatattaattggagtgaaggtagcccgatgtatatagattccgtgaaggaccgattcaccatctccagagacaacgccagaaattcgctgtatcttcagatgaacagcctgaaatctgaggacacggccgtttattactgtgcagcgcggaggttcggtcaggggtatgagtactggggccaggggacccaggtcaccgtctcctca

SEQ ID NO: 86 - DNA, 363 bp, synthetic construct
  source 1..363: mol_type = other DNA
gatgtgcagctggtggagtctgggggaggcttggtgcaggctggggggtctctgagactctcctgtggagtctctgggaacatcttcagtatgaatctcatgggctggtaccgccaacctccagggaaggagcgcgagtgggtcgcactaattactaaagatgggagcacaaattatgaagactccgtgaagggccgattcaccatctccagggacaacgccaagaacacggtgtctctgcaaatgaacagcctgaaacctgaggacacagccgtctactactgtaatgcccaggatggtagtggctggattcgggcgtggggctgggactggggccagggaacccaggtcaccgtctcctca